(19) United States
(12) Patent Application Publication
(10) Pub. No.: US 2013/0078215 A1
(43) Pub. Date: Mar. 28, 2013

(54) USES OF SELECTIVE INHIBITORS OF HDAC8 FOR TREATMENT OF INFLAMMATORY CONDITIONS

(71) Applicant: PHARMACYCLICS, INC., Sunnyvale, CA (US)

(72) Inventors: Joseph J. Buggy, Mountain View, CA (US); Sriram Balasubramanian, San Carlos, CA (US); Susanne M. Steggerda, San Francisco, CA (US)

(73) Assignee: PHARMACYCLICS, INC., Sunnyvale, CA (US)

(21) Appl. No.: 13/668,170

(22) Filed: Nov. 2, 2012

Related U.S. Application Data
(63) Continuation of application No. 11/940,232, filed on Nov. 14, 2007.
(60) Provisional application No. 60/954,777, filed on Aug. 8, 2007.
(60) Provisional application No. 60/944,409, filed on Jun. 15, 2007.
(60) Provisional application No. 60/911,857, filed on Apr. 13, 2007.
(60) Provisional application No. 60/865,825, filed on Nov. 14, 2006.

(51) Int. Cl. C07D 209/10; C07D 403/06; C07D 209/08; C07D 209/12; A61K 45/06; A61K 31/405; A61K 31/454; A61K 31/5377; A61K 31/4245; C07D 401/06; C07D 413/06

(52) U.S. Cl. CPC C07D 209/10; C07D 401/06; C07D 403/06; C07D 209/08; C07D 209/12; C07D 413/06; A61K 31/405; A61K 31/454; A61K 31/5377; A61K 31/4245; A61K 45/06
USPC 424/85.2; 514/415; 514/323; 514/235.5; 514/414; 514/364; 514/154; 514/24; 424/134.1; 424/85.5

(57) ABSTRACT

Described herein are methods for treating a subject suffering from an inflammatory, autoimmune, or heteroimmune condition by administering to the subject a pharmaceutical composition containing a therapeutically effective amount of a compound that is a selective inhibitor of histone deacetylase 8. Also described herein are methods for decreasing secretion of pro-inflammatory cytokines by administering an HDAC8-selective inhibitor compound. Further described herein are methods for predicting responsiveness to treatments for inflammatory conditions. Methods for predicting efficacy of treatments for inflammatory conditions are also described.

### RELATED APPLICATIONS

This application is a continuation of U.S. application Ser. No. 11/940,232, which claims benefit of U.S. Provisional Application No. 60/865,825, entitled “Therapeutic Uses of Selective Inhibitors of HDAC8” filed Nov. 14, 2006, U.S. Provisional Application No. 60/911,857, entitled “Therapeutic Uses of Selective Inhibitors of HDAC8” filed Apr. 13, 2007, U.S. Provisional Patent Application No. 60/944,409, entitled “Therapeutic Uses of Selective Inhibitors of HDAC8” filed Jun. 15, 2007, and U.S. Provisional Application No. 60/954,777, entitled “Therapeutic Uses of Selective Inhibitors of HDAC8” filed Aug. 8, 2007, each of which are hereby incorporated by reference.

### FIELD OF THE INVENTION

Described herein are methods for using selective inhibitors of histone deacetylase 8 (HDAC8) in the treatment of inflammatory conditions.

### BACKGROUND

Histone deacetylases (HDACs) were originally identified as proteins that catalyze the removal of acetyl groups from histones, proteins that organize and modulate the structure of chromatin in nucleosomes. HDAC-mediated deacetylation of chromatin-bound histones and other acetylated protein substrates (e.g., tubulin) plays a key role in cell signaling. Importantly, HDACs have been linked to cancer. To date, eleven major HDAC isoforms have been described (HDACs 1-11). Certain HDACs are overexpressed in, e.g., prostate cancer (HDAC1), colon cancers (HDAC3), and breast cancers (HDAC6). Indeed, HDAC activity is increasingly recognized as playing an important role in the onset and progression of cancer, as well as other health conditions.

### SUMMARY OF THE INVENTION

Described herein are methods for treating (including alleviating symptoms, preventing spread, delaying progression, and/or curing) inflammatory conditions in which the treatment comprises administering a selective inhibitor of histone deacetylase 8 (abbreviated as HDAC8) activity. Also described herein are methods for decreasing secretion of a pro-inflammatory cytokine, in which the treatment comprises administering a selective inhibitor of HDAC8 activity. Further described herein are methods for determining whether a particular inflammatory disorder is treated using a selective inhibitor of HDAC8. Further described herein are methods for assessing and/or predicting the effectiveness of a particular HDAC8 inhibitor (including the dose levels and/or dose schedules) for or in the treatment of an inflammatory condition.

In one aspect are methods for treating an inflammatory condition, comprising administering to a subject in need a composition containing a therapeutically effective amount of a selective inhibitor of histone deacetylase 8 activity.

In one embodiment of such methods, the inflammatory condition is a skin inflammatory condition, e.g., allergic contact dermatitis, urticarial dermatitis, psoriasis, eczema, erythroderma, mycosis fungoides, pyoderma gangrenosum, erythema multiforme, rosacea, discoid lupus, cutaneous sarcoid, onychomycosis, or acne. In some embodiments the HDAC inhibitor compound is administered locally (e.g., topically) to treat the inflammatory condition.

In another embodiment of such methods, the inflammatory condition is an autoimmune condition, e.g., rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia.

In another embodiment of such methods, the inflammatory condition is a heteroimmune condition, e.g., graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, or allergic rhinitis.

In another embodiment of such methods, the inflammatory condition is rheumatoid arthritis, juvenile RA (aka juvenile idiopathic arthritis) or psoriasis. In another embodiment of such methods, the inflammatory condition is gout or pseudogout. In another embodiment of such methods, the inflammatory condition is discoid lupus or subacute lupus.

In another embodiment of such methods, the secretion of IL-1β in a sample taken from the subject is inhibited by at least 40%, and/or the swelling on the skin of the subject decreases by at least 30% after administering the therapeutically effective amount of the selective inhibitor of histone deacetylase 8 activity.

In another embodiment of such methods, the subject is refractory or intolerant to at least one other treatment for an inflammatory condition.

In another embodiment of such methods, the composition is administered in combination with an additional anti-inflammatory agent.

In another embodiment of such methods, the anti-inflammatory agent is immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

In another embodiment of such methods, the anti-inflammatory agent is non-steroidal anti-inflammatory drugs (NSAIDs) and corticosteroids (glucocorticoids). NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, CS-502, JTE-522, L-745,337 and NS398). Compounds that have been described as selective COX-2 inhibitors and are therefore useful in the methods or pharmaceutical compositions described herein include, but are not limited to, celecoxib, rofecoxib, lumiracoxib, etoricoxib, valdecoxib, and parecoxib, or a pharmaceutically acceptable salt thereof. Corticosteroids, include, but are not limited to: betamethasone (Celestone®), prednisone (Deltasone®), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

In another embodiment of such methods, the composition is administered systemically, locally, or topically.

In another embodiment of such methods, the composition is administered topically.

In another embodiment of such methods, the selective inhibitor of HDAC8 is a 1,3-disubstituted-1H-indole-6-carboxylic acid hydroxyamide compound, wherein the substituent at the 1-position is —X²—R² and the substituent at the 3-position is R³, wherein:
X² is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene; —C(═O)—, and —C(═O)—C₁-C₆alkylene;
R² is a substituted or unsubstituted group selected from among aryl, heteroaryl, cycloalkyl, and heterocycloalkyl;
where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is hydrogen, halogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁶—R⁶;
X⁶ is a C₁-C₆alkylene, C₁-C₆fluoroalkylene, C₂-C₆alkenylene, C₂-C₆heteroalkylene;
R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁷—R⁷
X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁷ together with the N atom to which they are attached form a 5-, 6-, or 7-membered heterocycloalkyl;
   or an active metabolite, pharmaceutically acceptable solvate, pharmaceutically acceptable salt, pharmaceutically acceptable N-oxide, or pharmaceutically acceptable prodrug thereof.

In another embodiment of such methods, the selective inhibitor of HDAC8 is a compound selected from among:
1-(3,4-dichloro-phenylmethyl)-1H-indole-6-carboxylic acid hydroxyamide (Compound 1); 1-(2-methyl-phenylmethyl)-1H-indole-6-carboxylic acid hydroxyamide (Compound 2); 1-(3,4,5-trimethoxy-phenylmethyl)-1H-indole-6-carboxylic acid hydroxyamide (Compound 3); 1-(3-fluoro-phenylmethyl)-1H-indole-6-carboxylic acid hydroxyamide (Compound 4); 1-(3-methyl-phenylmethyl)-1H-indole-6-carboxylic acid hydroxyamide (Compound 5); 1-(benzyl)-1H-indole-6-carboxylic acid hydroxyamide (Compound 6); 1-(3,5-dimethoxy-phenylmethyl)-1H-indole-6-carboxylic acid hydroxyamide (Compound 7); 1-(1-methyl-1-phenylmethyl)-1H-indole-6-carboxylic acid hydroxyamide (Compound 8); 1-(4-fluoro-phenylmethyl)-1H-indole-6-carboxylic acid hydroxyamide (Compound 9); 1-(2-fluoro-phenylmethyl)-1H-indole-6-carboxylic acid hydroxyamide (Compound 10); 1-(2-chloro-phenylmethyl)-1H-indole-6-carboxylic acid hydroxyamide (Compound 11); 1-(3-methoxy-phenylmethyl)-1H-indole-6-carboxylic acid hydroxyamide (Compound 12); 1-(naphth-2-ylmethyl)-1H-indole-6-carboxylic acid hydroxyamide (Compound 13); 1-(3-phenylpropyl)-1H-indole-6-carboxylic acid hydroxyamide (Compound 14); 1-(cyclohexylmethyl)-1H-indole-6-carboxylic acid hydroxyamide (Compound 15); 1-[1-(phenyl)-propen-3-yl]-1H-indole-6-carboxylic acid hydroxyamide (Compound 16); 1-[4-(trifluoromethoxy)-phenylmethyl]-1H-indole-6-carboxylic acid hydroxyamide (Compound 17); 1-(4-chloro-phenylmethyl)-1H-indole-6-carboxylic acid hydroxyamide (Compound 18); 1-(benzo[2,1,3]oxadiazol-5-ylmethyl)-1H-indole-6-carboxylic acid hydroxyamide (Compound 19; 1-(4-methyl-phenylmethyl)-1H-indole-6-carboxylic acid hydroxyamide (Compound 20); 1-(3-fluoro-4-methoxy-phenylmethyl)-1H-indole-6-carboxylic acid hydroxyamide (Compound 21); 1-[4-(difluoromethoxy)-phenylmethyl]-1H-indole-6-carboxylic acid hydroxyamide (Compound 22); 1-(4-methoxy-phenylmethyl)-1H-indole-6-carboxylic acid hydroxyamide (Compound 23); 1-(phenethyl)-1H-indole-6-carboxylic acid hydroxyamide (Compound 24); 1-(3-chloro-phenylmethyl)-1H-indole-6-carboxylic acid hydroxyamide (Compound 25); 1-[N-(t-butoxycarbonyl)piperidin-4-ylmethyl]-1H-indole-6-carboxylic acid hydroxyamide (Compound 26); 1-(piperidin-4-ylmethyl)-1H-indole-6-carboxylic acid hydroxyamide (Compound 27); 1-(N-methylsulfonyl-3-aminobenzyl)-1H-indole-6-carboxylic acid hydroxyamide (Compound 28); 3-(Dimethylaminomethyl)-1-(4-methoxybenzyl)-1H-indole-6-carboxylic acid hydroxyamide (Compound 29); 3-(N-Morpholinomethyl)-1-(4-methoxybenzyl)-1H-indole-6-carboxylic acid hydroxyamide (Compound 30); 3-(N-Pyrrolidinomethyl)-1-(4-methoxybenzyl)-1H-indole-6-carboxylic acid hydroxyamide (Compound 31); 3-(N-Benzylaminomethyl)-1-(4-methoxybenzyl)-1H-indole-6-carboxylic acid hydroxyamide (Compound 32); and 3-(Ethyl)-1-(4-methoxybenzyl)-1H-indole-6-carboxylic acid hydroxyamide (Compound 33).

In other embodiments of such methods, the selective inhibitor of HDAC8 is a 1,3-disubstituted-1H-indole-5-carboxylic acid hydroxyamide compound, wherein the substituent at the 1-position is R⁴ and the substituent at the 3-position is —X⁵—R⁵, wherein:
R⁴ is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁸—R⁸;
X⁸ is a C₂-C₆alkylene, C₂-C₆fluoroalkylene, C₂-C₆alkenylene, or C₂-C₆heteroalkylene;
R⁸ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁹—R⁹;
X⁹ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or

R^{a} and R⁹ together with the N atom to which they are attached form a 5-, 6-, or 7-membered heterocycloalkyl;
X⁵ is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene, —C(═O)—, and —C(═O)—C₁-C₆alkylene;
R⁵ is a substituted or unsubstituted group selected from among aryl, heteroaryl, C₃-C₈cycloalkyl, and heterocycloalkyl;
where if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl; or an active metabolite, pharmaceutically acceptable solvate, pharmaceutically acceptable salt, pharmaceutically acceptable N-oxide, or pharmaceutically acceptable prodrug thereof.

In other embodiments of such methods, the selective inhibitor of HDAC8 is selected from among:
1-methyl-3-(4-nitro-phenylmethyl)-1H-indole-5-carboxylic acid hydroxyamide (Compound 34);
1-ethyl-3-(phenylmethyl)-1H-indole-5-carboxylic acid hydroxyamide (Compound 35);
1-methyl-3-[4-(phenylcarbonylamino)-phenylmethyl]-1H-indole-5-carboxylic acid hydroxyamide (Compound 36);
1-isopropyl-3-(phenylmethyl)-1H-indole-5-carboxylic acid hydroxyamide (Compound 37);
1-methyl-3-(4-amino-phenylmethyl)-1H-indole-5-carboxylic acid hydroxyamide (Compound 38);
1-methyl-3-(4-fluoro-phenylmethyl)-1H-indole-5-carboxylic acid hydroxyamide (Compound 39);
1-phenyl-3-(phenylmethyl)-1H-indole-5-carboxylic acid hydroxyamide (Compound 40); and
1-methyl-3-[4-(t-butoxycarbonyl)piperazin-1-ylmethyl]-1H-indole-5-carboxylic acid hydroxyamide (Compound 41).

In another aspect are methods for decreasing secretion of a pro-inflammatory cytokine, comprising administering to a subject in need a therapeutically effective amount of a selective inhibitor of histone deacetylase 8 activity.

In one embodiment of such methods, the pro-inflammatory cytokine is IL-1β, TNFα, IL-6, MCP-1, or MIP-1a. Chemokines are small proteins of MW 8-10 kDa. There are at least 50 chemokines and ˜19 chemokine receptors involved in a variety of processes, including inflammation, hematopoiesis, angiogenesis, and cancer. Chemokines are made by a variety of cells either in response to a stimulus or in a constitutive manner. All chemokines are secreted proteins and are produced as a precursor molecule with a hydrophobic signal peptide. Chemokines exert effects on target cells by binding to specific G-protein coupled receptors, which then causes a cascade of signal transduction events.

In one embodiment, the chemokine is MCP-1. Monocyte chemotactic protein 1 (MCP-1) is a member of the CC family of chemokines and binds to the CCR-2 receptor. MCP-1 attracts monocytes and activated natural killer and T cells. MCP-1 is mainly considered to be involved in angiogenesis, atherosclerosis, and inflammation. MCP-1 is pro-angiogenic in that it causes chemotaxis of endothelial cells and induces blood vessel formation in model systems. Knockout experiments indicate a role in atherosclerosis and multiple sclerosis. MCP-1 truncation mutant has shown promise in an arthritis model. In some embodiments, HDAC8-selective inhibitors (including those disclosed herein) are used for the treatment of inflammation and other diseases/conditions associated with MCP-1 secretion.

In another embodiment, the chemokine is MIP-1a. Macrophage inflammatory protein 1a (MIP-1a) is a CC chemokine and binds to the CCR-1 and CCR-5 receptors. MIP-1a is chemotactic for monocytes, T cells, and dendritic cells. The process MIP-1a is most associated with is inflammation. Homozygous CCR-5 gene variant confers resistance to HIV infection and blockade of this receptor is a potential treatment for HIV. In another embodiment, HDAC8-selective inhibitors (including those disclosed herein) are used for the treatment of inflammation and other diseases/conditions associated with MIP-1a secretion.

In another embodiment of such methods, the pro-inflammatory cytokine is IL-1β.

In another aspect are methods for predicting responsiveness to a treatment for an inflammatory condition, comprising: determining the level of histone deacetylase 8 activity in a biological sample from a subject having the inflammatory condition, and providing information that a higher level of the histone deacetylase 8 activity is indicative of the subject's higher likelihood of responsiveness to a composition containing a selective inhibitor of histone deacetylase 8 activity.

In one embodiment of such methods, the selective inhibitor of HDAC8 is a 1,3-disubstituted-1H-indole-6-carboxylic acid hydroxyamide compound, wherein the substituent at the 1-position is —X²—R² and the substituent at the 3-position is R³, wherein:
X² is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene; —C(═O)—, and —C(═O)—C₁-C₆alkylene;
R² is a substituted or unsubstituted group selected from among aryl, heteroaryl, cycloalkyl, and heterocycloalkyl;
where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is hydrogen, halogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁶—R⁶;
X⁶ is a C₁-C₆alkylene, C₁-C₆fluoroalkylene, C₂-C₆alkenylene, C₂-C₆heteroalkylene;
R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁷—R⁷
X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or

R^{a} and R⁷ together with the N atom to which they are attached form a 5-, 6-, or 7-membered heterocycloalkyl;

or an active metabolite, pharmaceutically acceptable solvate, pharmaceutically acceptable salt, pharmaceutically acceptable N-oxide, or pharmaceutically acceptable prodrug thereof.

In another embodiment of such methods, the selective inhibitor of HDAC8 is a 1,3-disubstituted-1H-indole-5-carboxylic acid hydroxyamide compound, wherein the substituent at the 1-position is R⁴ and the substituent at the 3-position is —X⁵—R⁵, wherein:
R⁴ is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁸—R⁸;
X⁸ is a C₂-C₆alkylene, C₂-C₆fluoroalkylene, C₂-C₆alkenylene, or C₂-C₆heteroalkylene;
R⁸ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁹—R⁹;
X⁹ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁹ together with the N atom to which they are attached form a 5-, 6-, or 7-membered heterocycloalkyl;
X⁵ is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene, —C(═O)—, and —C(═O)—C₁-C₆alkylene;
R⁵ is a substituted or unsubstituted group selected from among aryl, heteroaryl, C₃-C₈cycloalkyl, and heterocycloalkyl;
where if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;

R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl; or an active metabolite, pharmaceutically acceptable solvate, pharmaceutically acceptable salt, pharmaceutically acceptable N-oxide, or pharmaceutically acceptable prodrug thereof.

In another embodiment of such methods, the level of histone deacetylase 8 activity is determined by measuring a component of the IL-1β secretory pathway, including but not limited to measuring the activity or levels of IL-1β, MCP1, Mip1a or TNFα. Such methods of measuring the activity of levels of IL-1β, MCP1, Mip1α or TNFα include ELISA, Western blot or Taqman assays.

In another embodiment of such methods, the level of histone deacetylase 8 activity is determined by measuring a component of the IL-1β secretory pathway but not by measuring interleukin converting enzyme or phospholipase A₂ enzyme activity, or IL-1β protein levels.

In another aspect are methods for predicting efficacy of a treatment for an inflammatory condition comprising: administering to a subject having an inflammatory condition a composition containing a selective inhibitor of histone deacetylase 8 activity; monitoring the subject's histone deacetylase 8 activity for an increase or decrease in activity; and utilizing the patient's histone deacetylase 8 activity as an indication for the amount of the next dosage of the composition.

In one embodiment of such methods, the selective inhibitor of HDAC 8 is a 1,3-disubstituted-1H-indole-6-carboxylic acid hydroxyamide compound, wherein the substituent at the 1-position is —X²—R² and the substituent at the 3-position is R³, wherein:
X² is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene; —C(═O)—, and —C(═O)—C₁-C₆alkylene;
R² is a substituted or unsubstituted group selected from among aryl, heteroaryl, cycloalkyl, and heterocycloalkyl;
where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is hydrogen, halogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁶—R⁶;
X⁶ is a C₁-C₆alkylene, C₁-C₆fluoroalkylene, C₂-C₆alkenylene, C₂-C₆heteroalkylene;
R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁷—R⁷
X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁷ together with the N atom to which they are attached form a 5-, 6-, or 7-membered heterocycloalkyl;
   or an active metabolite, pharmaceutically acceptable solvate, pharmaceutically acceptable salt, pharmaceutically acceptable N-oxide, or pharmaceutically acceptable prodrug thereof.

In another embodiment of such methods, the selective inhibitor of HDAC8 is a 1,3-disubstituted-1H-indole-5-carboxylic acid hydroxyamide compound, wherein the substituent at the 1-position is R⁴ and the substituent at the 3-position is —X⁵—R⁵, wherein:
R⁴ is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁸—R⁸;
X⁸ is a C₂-C₆alkylene, C₂-C₆fluoroalkylene, C₂-C₆alkenylene, or C₂-C₆heteroalkylene;
R⁸ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁹—R⁹;
X⁹ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁹ together with the N atom to which they are attached form a 5-, 6-, or 7-membered heterocycloalkyl;
X⁵ is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene, —C(═O)—, and —C(═O)—C₁-C₆alkylene;
R⁵ is a substituted or unsubstituted group selected from among aryl, heteroaryl, C₃-C₈cycloalkyl, and heterocycloalkyl;
where if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl; or an active metabolite, pharmaceutically acceptable solvate, pharmaceutically acceptable salt, pharmaceutically acceptable N-oxide, or pharmaceutically acceptable prodrug thereof.

In another embodiment of such methods, the method further comprises adjusting the dosage of the composition administered to the subject.

In another embodiment of such methods, the monitoring of the subject's histone deacetylase 8 activity comprises measuring a component of the IL-1β secretory pathway but not by measuring interleukin converting enzyme or phospholipase A₂ enzyme activity, or IL-1β protein levels.

Other features, objects, and advantages will be apparent from the description and from the claims.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B are illustrative panels of photomicrographs of normal human tissue sections stained for HDAC8 expression using an anti-HDAC8 antibody.

FIG. 2 is an illustrative panel of photomicrographs of plasma cells found in various human tissues and stained for HDAC8 expression with an anti-HDAC8 antibody.

FIG. 3 is an illustrative immunoblot showing HDAC8 expression in a series of cell lines. For each cell line, Hsc 70 expression is also shown as a normalization control for apparent HDAC8 expression levels.

FIG. 4 is an illustrative bar graph showing the effect of RNAi knock-down of HDAC 8 on apoptosis in HeLa cells.

FIG. 5 is an illustrative panel of scatter plots showing the effect of the HDAC8-selective inhibitor compound, Compound 23, on cell proliferation in the cell line HCT116 and in normal human peripheral blood mononuclear cells.

FIG. 6 is an illustrative bar graph showing the dose-dependent inhibition of LPS/ATP-induced IL-1β secretion from human PBMCs in culture by an HDAC8-selective inhibitor compound (Compound 23).

FIG. 7 is an illustrative line graph showing the dose-dependent inhibition of LPS-induced IL-1β secretion from human PBMCs in culture after various incubation periods in the presence of an HDAC8-selective inhibitor compound (Compound 23).

FIG. 8 is an illustrative line graph showing the dose-dependent inhibition of LPS-induced IL-1β secretion from human PBMCs in culture by an HDAC8-selective inhibitor compound (Compound 23).

FIG. 9 is an illustrative graph showing the dose-dependent inhibition of ATP-induced IL-1β secretion from human PBMCs in culture after 4 or 18 hours in the presence of an HDAC8-selective inhibitor compound (Compound 23).

FIG. 10 is an illustrative bar graph showing the dose-dependent inhibition of LPS+ATP-induced IL-18 secretion from human PBMCs in culture by an HDAC8-selective inhibitor compound (Compound 23).

FIG. 11 is an illustrative line graph showing the dose-dependent inhibition of LPS-stimulated IL-6 and TNF-α secretion from human PBMCs in culture after 18 hours in the presence of an HDAC8-selective inhibitor compound (Compound 23).

FIG. 12 is an illustrative schematic depiction of experimental protocols for two in vivo models of allergic contact dermatitis, a type of inflammation.

FIG. 13 is an illustrative representative graph showing the levels of secreted cytokines in PBMC supernatant after 24 h of treatment with an HDAC8-selective inhibitor compound (Compound 23).

FIG. 14 is an illustrative representative bar graph showing the dose-dependent inhibition of LPS-induced MCP-1 secretion from human PBMCs in culture by an HDAC8-selective inhibitor compound (Compound 23).

FIG. 15 is an illustrative graph showing secretion of IL-1β and other pro-inflammatory cytokines to LPS-induced human PBMCs in culture with various concentrations of Compound 23 and lipopolysaccharide (LPS).

FIG. 16 is an illustrative graph showing IL-1β secretion in LPS-induced primary human monocytes in culture with various concentrations of Compound 23 and lipopolysaccharide (LPS).

FIG. 17 is an illustrative graph showing IL-1β secretion in LPS-induced THP-1 monocyte cells in culture with various concentrations of Compound 23 and lipopolysaccharide (LPS).

FIG. 18 presents illustrative graphs showing LPS and LPS+ATP induced IL-1β secretion in culture with various concentrations of Compound 23 and lipopolysaccharide (LPS).

FIG. 19 is an illustrative graph showing Caspase-1 and TNF-α converting enzyme (TACE) inhibition after incubation with Compound 23.

FIG. 20 is an illustrative micrograph showing IL-1β levels of human primary monocytes pretreated with various concentrations of Compound 23 before stimulation with 10 ng/mL LPS for an additional 15 hours.

FIG. 21 is an illustrative graph showing LPS-induced secretion of uncleaved pro-IL-1β in culture with various concentrations of Compound 23 and LPS.

FIG. 22 is an illustrative graph showing ear-swelling induced by oxazolone in BALB/c mice after incubation in various concentrations of Compound 23.

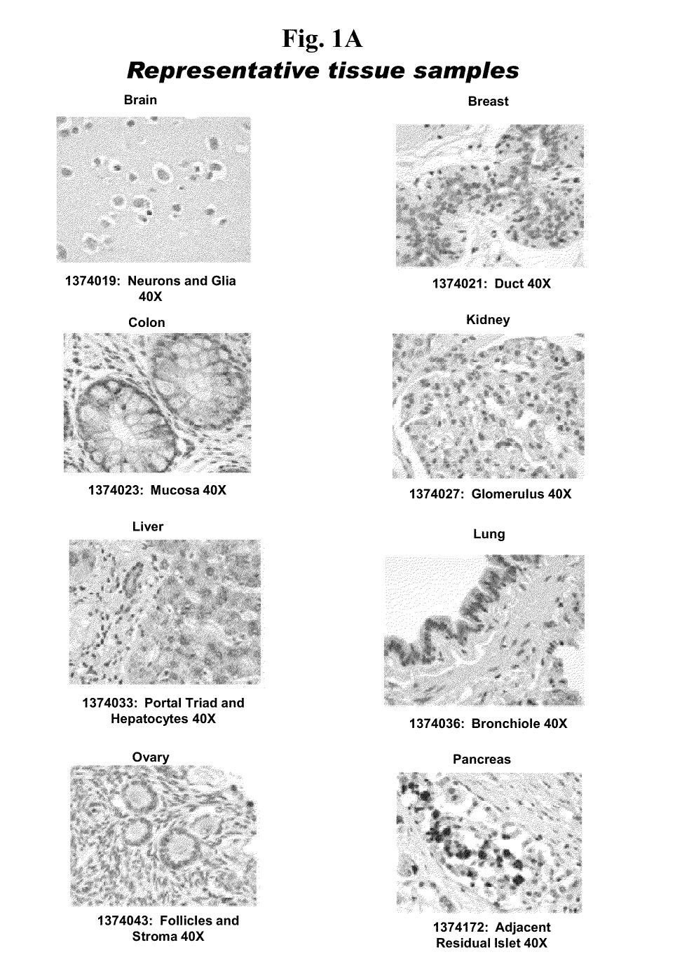

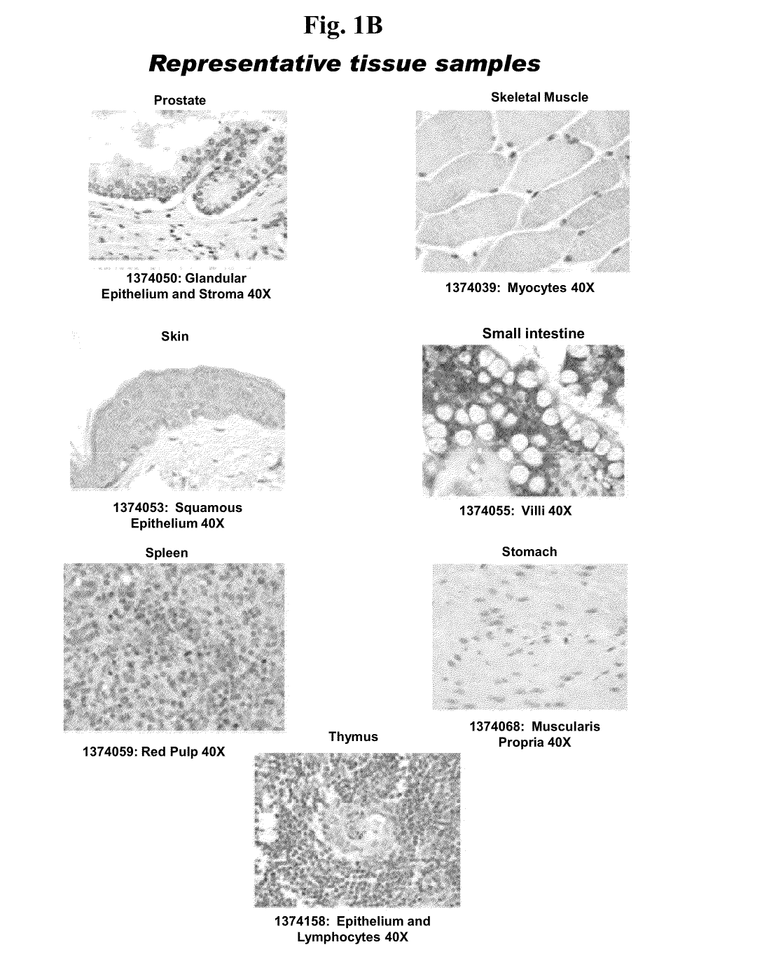

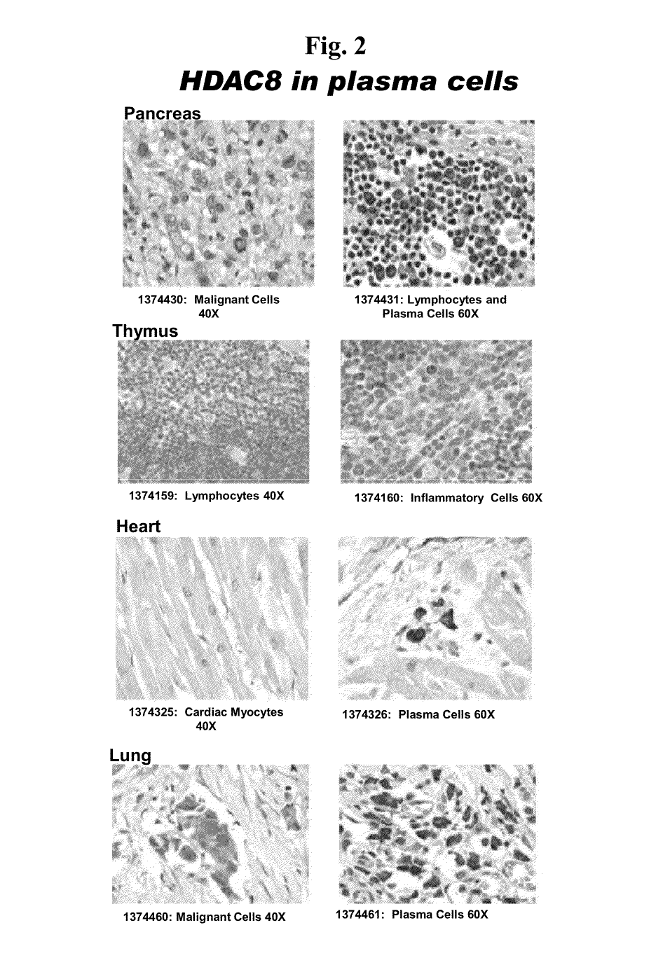

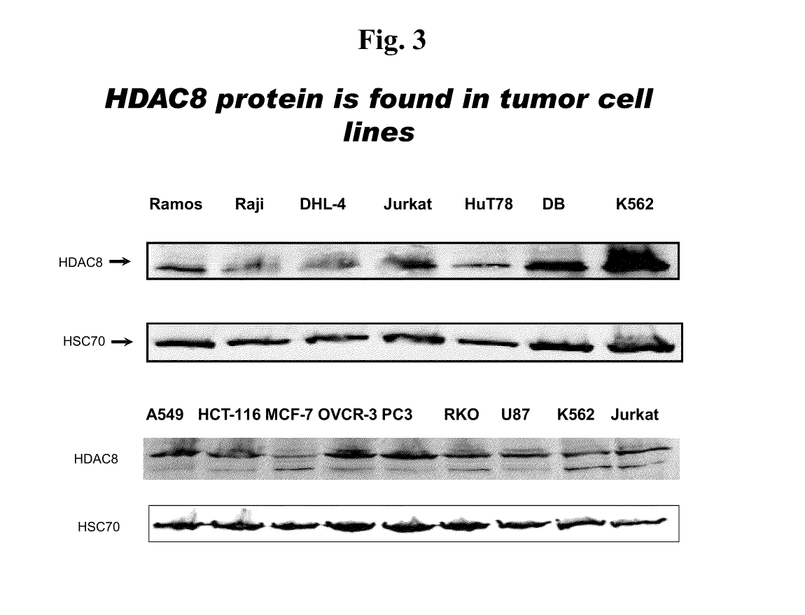

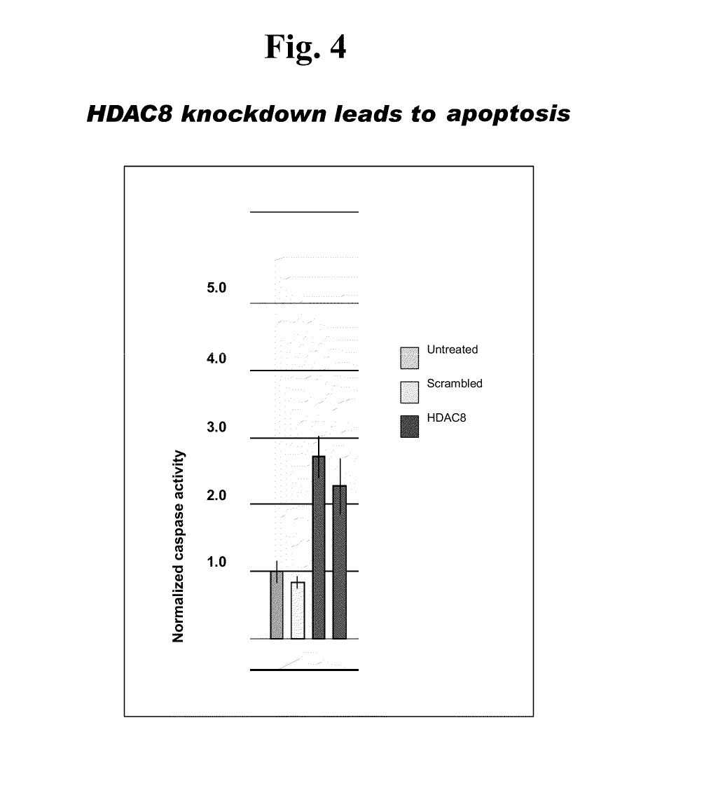

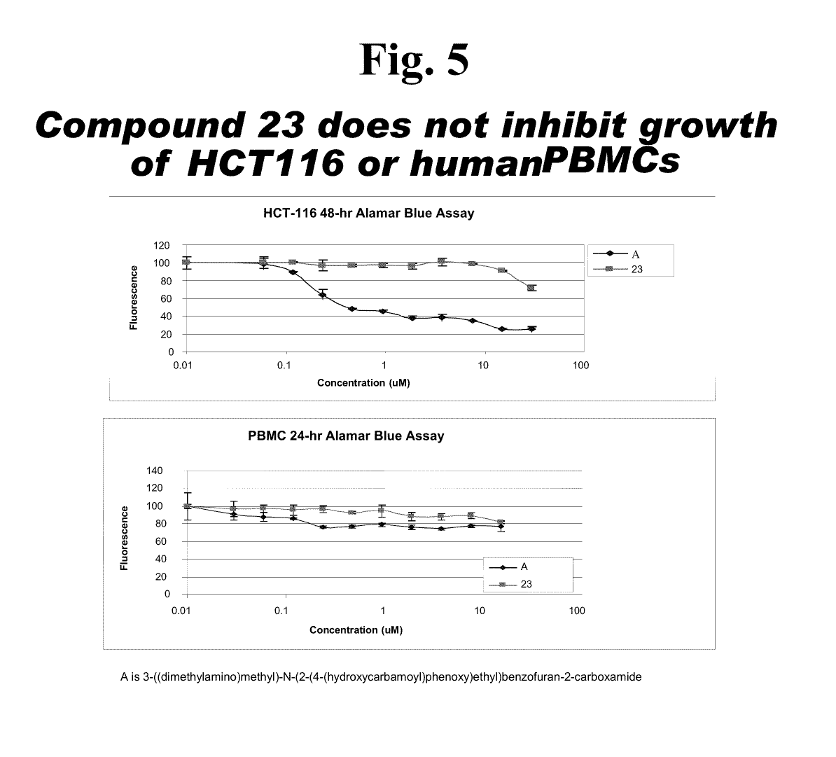

### DETAILED DESCRIPTION OF THE INVENTION

Covalent modification of histone proteins through acetylation and deacetylation is an important determinant of chromatin structure and a regulator of gene expression. Acetylation of histone proteins occurs on lysine residues near the N-termini of these proteins. In conjunction with other modifications of histone proteins and DNA, the acetylation state of histones determines whether the chromatin is in a condensed, transcriptionally silent state or in a form more accessible to the transcription machinery of the cell. In general, hyperacetylation of histone proteins is associated with transcriptional activation of genes. The steady-state histone acetylation level arises from the opposing action of histone acetyltransferase (HAT) and histone deacetylase (HDAC) enzymes.

Histone deacetylases (HDACs) catalyze the removal of acetyl groups from lysine ε-amino groups near the N-termini of histones. This reaction promotes the condensation of chromatin, leading to repression of transcription.

HDAC inhibitors (HDIs) modify gene expression positively or negatively in a cell- and gene-specific manner. HDIs increase the accumulation of acetylated histones, directly influencing chromatin structure and, thereby, the relationship of the nucleosome to gene promoter elements.

Histone deacetylase (HDAC) enzymes modulate gene expression through the deacetylation of acetylated lysine residues on histone proteins. They operate in biological systems as part of multiprotein corepressor complexes. Histone deacetylases have been grouped into three classes. Class I and class II histone deacetylases (HDACs) are zinc containing hydrolase enzymes. The division of the proteins into classes I and II is based on protein size, sequence similarity, and organization of the protein domains.

Members of class I are related to the yeast RPD3 gene product. Class I HDACs include: HDAC1 (GenBank Accession Number NP_{—}004955; Wolffe, A. P., Science 272, 5260, 371-372, 1996); HDAC2 (GenBank Accession Number NP_{—}001518; Furukawa, et al., Cytogenet. Cell Genet. 73; 1-2, 130-133, 1996); HDAC3 (GenBank Accession Number NP_{—}003874; Yang, et al., J. Biol. Chem. 272, 44, 28001-28007, 1997); HDAC8 (GenBank Accession Number NP_{—}060956; Buggy, et al., Biochem. J. 350 Pt 1, 199-205, 2000); HDAC11 (GenBank Accession Number NP_{—}079103; Gao, L. et al., J. Biol. Chem. 277, 28, 25748-25755, 2002).

HDAC8 is a 377 residue, 42 kDa protein localized to the nucleus of a wide array of tissues, as well as several human tumor cell lines. The wild-type form of full length HDAC8 is described in GenBank Accession Number NP 060956; Buggy, J. J. et al., Biochem. J., 350 (Pt 1), 199-205 (2000). The HDAC8 structure was solved with four different hydroxamate inhibitors bound (Somoza et al., Structure, 2004, 12, 1325).

A “selective HDAC8 inhibitor,” as used herein, refers to a compound that has an IC₅₀ for inhibition of HDAC8 acetyltransferase activity that is at least about 5 fold to more than about 500 fold lower than for the acetyltransferase activity of another HDAC. In some embodiments, the selective HDAC8 inhibitor has an IC₅₀ for HDAC8 acetyltransferase activity that is about 5, about 10, about 50, about 150, about 200, about 250, about 300, about 350, about 400, about 450 or more than about 500 fold lower than the IC₅₀ for acetyltransferase activity of another HDAC. In one embodiment, the selective HDAC8 inhibitor has an IC₅₀ for HDAC8 activity that is at least about 10 fold lower than the IC₅₀ for HDAC1, HDAC2, HDAC3, HDAC6, HDAC10, and HDAC11.

Described herein are methods for treating inflammatory conditions in which the treatment comprises administering a selective inhibitor of histone deacetylase 8 (abbreviated as HDAC8) activity. Also described herein are methods for decreasing secretion of a pro-inflammatory cytokine, in which the treatment comprises administering a selective inhibitor of HDAC8 activity. Further described herein are methods for determining whether a particular inflammatory disorder is treated using a selective inhibitor of HDAC8. Further described herein are methods for assessing and/or predicting the effectiveness of a particular HDAC8 inhibitor (including the dose levels and/or dose schedules) for or in the treatment of an inflammatory condition.

The methods described herein include administering a pharmaceutical composition containing a selective HDAC8 inhibitor in a quantity sufficient to decrease HDAC8 deacetylase activity in vivo by a therapeutically effective amount.

Methods for determining HDAC activity in vivo or in vitro include, e.g., Kim et al. (2006), Methods Mol Biol., 325:273-283.

Selective HDAC8 inhibitors, as described herein, decrease secretion, in peripheral blood mononuclear cells, of the cytokines IL-1β, tumor necrosis factor β (TNF-α), interleukin 6, (IL-6), IL-18, monocyte chemotactic protein 1 (MCP-1), and macrophage inflammatory protein 1a (MIP-1a), all of which play important roles in the immune response and inflammation. Accordingly, in some embodiments, selective HDAC8 inhibitors are useful for inhibiting inflammatory immune responses.

The methods described herein are useful for treating a subject suffering from one or more conditions, including, but not limited to, any the following described below.

Unless defined otherwise, all technical and scientific terms used herein have the same meaning as is commonly understood by one of skill in the art to which the claimed subject matter belongs.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### Use of Selective HDAC8 Inhibitors for Treating Cytokine-Modulated Health Conditions

In some embodiments, a subject is administered a therapeutically effective amount of a selective HDAC8 inhibitor to decrease secretion of one or more inflammatory cytokines (e.g., IL-1β).

In some embodiments a selective HDAC8 inhibitor compound is administered to a subject to decrease the systemic levels of one or more inflammatory cytokines including, e.g., IL-1β, IL-6, IL-18, TNF-α, MCP-1, or MIP-1a.

As described herein, selective HDAC8 inhibitor compounds described herein reduce the secretion of proinflammatory cytokines including but not limited to interleukin-1 beta (IL-β). Thus, HDAC8 is the HDAC enzyme involved in cytokine secretion. The use of selective HDAC8 inhibitor compounds provides a method of reducing cytokine secretion with reduced toxicity, due to the selective inhibition of one HDAC isoform (vs. the use of pan-HDAC inhibitors that inhibit all of the HDAC isoforms).

Selective HDAC8 inhibitor compounds described herein inhibit, in a dose dependent fashion, lipopolysaccharide (LPS) and/or ATP stimulated secretion of IL-1β from purified human peripheral blood mononuclear cells (PBMCs) as well as from the monocyte cell line THP-1. In some embodiments, the EC₅₀ for inhibition ranges from 0.5 micromolar to 5 micromolar.

The production and secretion of IL-1β is via a non-classical pathway of protein secretion, involving potassium efflux, the autocatalytic processing of procaspase-1, the cleavage by active caspase-1 of the IL-1β precursor, the influx of calcium ions, and the activation of specific phospholipases including PLA-2. In some embodiments, selective HDAC8 inhibitor compounds described herein inhibit one or more steps in this secretory pathway.

As described herein, selective HDAC8 inhibitors are used to treat diseases or conditions that are mediated or linked to IL-1β secretion and activity. In certain autoimmune diseases or conditions, IL-1β is contributes to the signs and symptoms of the diseases or conditions (for examples of such Burger et al., Best Practice & Research Clinical Rheumatology, Vol. 20, No. 5, pp. 879-896, 2006; Dayer et al., Current Opinions in Rheum., 2001, 13:170-176; Abramson et al., Rheumatology, 2002; 41; 972-980); selective HDAC8 inhibitor compounds are used to treat such diseases or conditions. As described herein, selective HDAC8 inhibitor compounds are used to inhibit IL-1β secretion and thus find utility in the treatment of diseases or conditions that are linked to IL-1β secretion and activity, which include, but are not limited to, osteoarthritis, rheumatoid arthritis, septic arthritis, gout, pseudogout, juvenile arthritis, Still's disease, Ankylosing spondylitis, systemic lupus erythematosus (SLE), Henoch-Schönlein purpura, psoriatic arthritis, reactive arthritis (Reiter's syndrome), hemochromatosis, hepatitis, Wegener's granulomatosis, Familial Mediterranean fever (FMF), HIDS (hyperimmunoglobulinemia D and periodic fever syndrome), TRAPS (TNF-alpha receptor associated periodic fever syndrome), inflammatory bowel disease, Crohn's Disease, ulcerative colitis, recurrent fever, anemia, leukocytosis, asthma, chronic obstructive pulmonary disease, myalgia; Adult Still's disease, Systemic-onset juvenile idiopathic arthritis, Lupus arthritis, Ankylosing spondylitis, familial Mediterranean fever (FMF), TNF receptor-associated periodic syndrome (TRAPS), hyperimmunoglobulinemia D with periodic fever syndrome (HIDS), Blau syndrome, FCAS, MWS, neonatal-onset multisystem inflammatory disease (NOMID) and cryopyrin-associated periodic syndrome (CAPS), familial cold autoinflammatory syndrome (FCAS); Muckle-Wells syndrome (MWS); neonatal-onset multisystem inflammatory disease (NOMID); chronic infantile neurologic, cutaneous, articular syndrome (CINCA); cryopyrin-associated periodic syndrome (CAPS); pyogenic sterile arthritis, pyoderma gangrenosum, and acne syndrome (PAPA).

In further embodiments, the methods described herein are used to treat an inflammatory disease, which includes, but is not limited to asthma, inflammatory bowel disease, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In yet other embodiments, the methods described herein are used to treat an inflammatory skin condition. Inflammatory skin conditions are those conditions of the skin in which inflammatory cells (e.g., polymorphonuclear neutrophils and lymphocytes) infiltrate the skin with no overt or known infectious etiology. Symptoms of inflammatory skin conditions generally include erythema (redness), edema (swelling), pain, pruritus, increased surface temperature and loss of function. As used herein, inflammatory skin conditions include, but are not limited to, allergic contact dermatitis, urticarial dermatitis, psoriasis, eczema and related conditions, insect bites, erythroderma, mycosis fungoides and related conditions, pyoderma gangrenosum, erythema multiforme, rosacea, onychomycosis, and acne and related conditions, but excluding psoriasis and its related conditions.

In some embodiments, the methods described herein are used to treat an autoimmune disease, which includes, but is not limited to, rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, and vulvodynia.

In some embodiments, the methods described herein are used to treat heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Chronic inflammation in patients has been linked to cancer development (Coussens et al., Nature, 420, 860-867, 2002). Cancers associated with chronic inflammation include, but are not limited to, lung, esophageal, gastric, pancreatic, cervical, bladder, prostate and colorectal cancers. The role of the inflammatory microenvironment as a causative factor in the etiology of cancer is also supported by findings that regular use of non-steroidal anti-inflammatory drugs (NSAIDs) is associated with a reduced incidence of colorectal, breast and gastric cancer. Pro-inflammatory cytokines are mediators of chronic inflammatory responses, and have effects on malignant processes.

Pro-inflammatory cytokines are involved in carcinogenesis and malignant transformation, tumor growth, invasion and metastasis. Persistent expression of proinflammatory cytokines, in or near tumors, exerts a range of effects, including but not limited to, increasing growth and invasiveness of the malignant cells, metastasis, tumorigenesis, to activation of immune-mediated mechanisms, leading to the destruction of tumor cells and inhibition of tumor growth. IL-1β-transfected tumor cells have been reported to fail to induce effective antitumor immune responses. In several human cancers, local IL-1β expression by the malignant cells or the microenvironment has been associated with aggressive tumor growth and poor prognosis.

In IL-1β-transfected fibrosarcoma cells, an up-regulation of MMP-2 and MMP-9 and TGFβ, genes that are involved in invasiveness, was observed, as opposed to the shut-off of these genes in IL-1α-transfected fibrosarcomas cells. IL-1β is thought to also enhance the invasiveness of already existing tumor cells by switching on angiogenesis and by the induction of inflammatory molecules, such as MMPs, heparanase, chemokines or integrins on the malignant cells or endothelial cells, leading to tumor dissemination and metastasis. IL-1β induces secretion of growth and invasiveness-promoting factors, e.g. matrix metalloproteinases and angiogenic factors (i.e. VEGF and bFGF and ELR-positive CXC chemokines, i.e. IL-8 and MCP-1). (Apte et al., seminars in Cancer Biology, vol. 12, 2002, 277-290).

Secreted IL-1β has been implicated in tumor growth and invasion Inhibition of IL-1β secretion, e.g. by using selective HDAC8 compounds, in malignant cells, or in the tumor's microenvironment provides a method for cancer therapy.

Thus in one embodiment, selective HDAC8 compounds described herein, are used in cancer therapy. In one embodiment, selective HDAC8 compounds described herein, are used in the treatment of sarcomas. In another embodiment, selective HDAC8 compounds described herein, are used in the treatment of sarcomas selected from among alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, malignant fibrous histiocytoma, neurofibrosarcoma, rhabdomyosarcoma, synovial sarcoma, askin's tumor, ewing's, malignant hemangioendothelioma, malignant schwannoma, osteosarcoma, chondrosarcoma.

Symptoms, diagnostic tests, and prognostic tests for each of the above-mentioned conditions are known. See, e.g., “Harrison's Principles of Internal Medicine©,” 16th ed., 2004, The McGraw-Hill Companies, Inc.

In various embodiments described herein, a subject suffers from more than one condition that is treated by administration of a therapeutically effective amount of a selective HDAC8 inhibitor composition. Thus, it is to be understood that the methods described herein are effective for treating a subject suffering from any combination of health conditions amenable to treatment by administration of a selective HDAC8 inhibitor composition. For example, in some embodiments, a subject suffering from a T-cell lymphoma also suffers from an inflammatory condition and vice versa.

### Examples of Selective HDAC8 Inhibitors

In one embodiment, provided herein is a 1,3-disubstituted-1H-indole-5-carboxylic acid hydroxyamide compound, wherein the substituent at the 1-position is R⁴ and the substituent at the 3-position is —X⁵—R⁵, wherein:
R⁴ is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁸—R⁸;
X⁸ is a C₂-C₆alkylene, C₂-C₆fluoroalkylene, C₂-C₆alkenylene, or C₂-C₆heteroalkylene;
R⁸ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁹—R⁹;
X⁹ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁹ together with the N atom to which they are attached form a 5-, 6-, or 7-membered heterocycloalkyl;
X⁵ is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene, —C(═O)—, and —C(═O)—C₁-C₆alkylene;
R⁵ is a substituted or unsubstituted group selected from among aryl, heteroaryl, C₃-C₈cycloalkyl, and heterocycloalkyl;
where if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
   or an active metabolite, pharmaceutically acceptable solvate, pharmaceutically acceptable salt, pharmaceutically acceptable N-oxide, or pharmaceutically acceptable prodrug thereof.

In some embodiments, substituents are selected from among from a subset listed herein. For example, in some embodiments, R⁴ is hydrogen, substituted or unsubstituted C₁-C₆alkyl, substituted or unsubstituted C₂-C₆alkenyl, substituted or unsubstituted C₁-C₆alkoxy, substituted or unsubstituted C₁-C₆heteroalkyl, substituted or unsubstituted phenyl, or —X⁸—R⁸; X⁸ is a C₂-C₆alkylene, C₂-C₆fluoroalkylene, C₂-C₆alkenylene, or C₂-C₆heteroalkylene; R⁸ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁹—R⁹; X⁹ is a bond, —O—, —S—, —NR^{a}—, —C(═O)—; R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl; R^{a} is selected from among hydrogen, C₁-C₆alkyl, C₂-C₆alkenyl, hydroxy, C₁-C₆alkoxy, and C₁-C₆heteroalkyl; or R^{a} and R⁹ together with the N atom to which they are attached form a 5-, or 6-membered heterocycloalkyl.

In other embodiments, R⁴ is hydrogen, substituted or unsubstituted C₁-C₆alkyl, substituted or unsubstituted C₁-C₆heteroalkyl, substituted or unsubstituted phenyl, or —X⁸—R⁸; X⁸ is a C₂-C₆alkylene; R⁸ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁹—R⁹; X⁹ is a bond, —O—, —S—, —NR^{a}—, —C(═O)—; R⁹ is hydrogen, C₁-C₆alkyl, C₁-C₆heteroalkyl, C₁-C₆haloalkyl, C₂-C₈heterocycloalkyl, heterocycloalkylalkyl, phenyl, phenylalkyl, heteroaryl, heteroarylalkyl; R^{a} is selected from among hydrogen, C₁-C₆alkyl, hydroxy, C₁-C₆alkoxy; or R^{a} and R⁹ together with the N atom to which they are attached form a 5-, or 6-membered heterocycloalkyl.

In some embodiments, R⁴ is selected from among hydrogen, methyl, ethyl, propyl, isopropyl, phenyl, and benzyl.

In some embodiments, X⁵ is a bond, or a substituted or unsubstituted group selected from among C₁-C₆alkylene, C₂-C₆alkenylene, C₁-C₆fluoroalkylene, and C₁-C₆heteroalkylene. In other embodiments, X⁵ is a bond, or a substituted or unsubstituted C₁-C₆alkylene. In some embodiments, X⁵ is —CH₂—, —CH₂CH₂—, —CH(CH₃)—, —(CH₂)₃₋₅ or —CH₂CH═CH—. In some embodiments, X⁵ is —CH₂—.

In some embodiments, R⁵ is a substituted or unsubstituted group selected from among phenyl, naphthyl, (heteroaryl containing 0-2 N atoms, 0-10 atoms, 0-1 S atoms), C₃-C₈cycloalkyl, and heterocycloalkyl containing 0-2 N atoms.

In some embodiments, if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₁-C₆alkoxy, C₁-C₆ fluoroalkoxy, substituted or unsubstituted C₁-C₆heteroalkyl, substituted or unsubstituted heterocycloalkyl, substituted or unsubstituted phenyl, and substituted or unsubstituted heteroaryl; R¹⁰ is hydrogen, or a substituted or unsubstituted group selected from among C₁-C₆alkyl, C₁-C₆fluoroalkyl, C₁-C₆heteroalkyl, phenyl, and heteroaryl; R¹¹ is a substituted or unsubstituted group selected from among C₁-C₆alkyl, C₁-C₆fluoroalkyl, phenyl, and heteroaryl.

In some embodiments, R⁵ is a substituted or unsubstituted group selected from among phenyl, naphthyl, (monocyclic heteroaryl containing 0-2 N atoms, 0-10 atoms, 0-1 S atoms), and C₂-C₈heterocycloalkyl containing 0-2 N atoms.

In some embodiments, R⁵ is selected from among phenyl, 2-methylphenyl, 3-methylphenyl, 4-methylphenyl, 3,4-dimethylphenyl, 2-fluorophenyl, 3-fluorophenyl, 4-fluorophenyl, 3,4-difluorophenyl, 2-chlorophenyl, 3-chlorophenyl, 4-chlorophenyl, 3,4-dichlorophenyl, 3-methoxyphenyl, 3-methoxyphenyl, 4-methoxyphenyl, 3,5-dimethoxyphenyl, 3,4,5-trimethoxyphenyl, naphth-2-yl, cyclopentyl, cyclohexyl, cycloheptyl, 2-(trifluoromethyl)-phenyl, 3-(trifluoromethyl)-phenyl, 4-(trifluoromethyl)-phenyl, 2-(trifluoromethoxy)-phenyl, 3-(trifluoromethoxy)-phenyl, 4-(trifluoromethoxy)-phenyl, benzo[2,1,3]oxadiazol-5-yl, 3-fluoro-4-methoxy-phenyl, 2-(difluoromethoxy)-phenyl, 3-(difluoromethoxy)-phenyl, 4-(difluoromethoxy)-phenyl, N-(t-butoxycarbonyl)piperidin-4-yl, piperidin-4-yl, N-methylsulfonyl-2-aminophenyl, N-methylsulfonyl-3-aminophenyl, N-methylsulfonyl-4-aminophenyl, N-phenylsulfonyl-2-aminophenyl, N-phenylsulfonyl-3-aminophenyl, N-phenylsulfonyl-4-aminophenyl, 2-nitrophenyl, 3-nitrophenyl, 4-nitrophenyl, 2-aminophenyl, 3-aminophenyl, 4-aminophenyl, 2-dimethylaminophenyl, 3-dimethylaminophenyl, 4-dimethylaminophenyl, N-acetyl-2-aminophenyl, N-acetyl-3-aminophenyl, N-acetyl-4-aminophenyl, 2-(phenylcarbonylamino)-phenyl, 3-(phenylcarbonylamino)-phenyl, and 4-(phenylcarbonylamino)-phenyl. In some embodiments, R⁵ is selected from among phenyl, 4-nitrophenyl, 4-aminophenyl, 4-(phenylcarbonylamino)-phenyl, 4-fluorophenyl, and 4-(t-butoxycarbonyl)piperazin-1-yl.

Any combination of the groups described above for the various variables is contemplated herein.

In another aspect, provided herein is a compound having a structure selected from among Formula (Ia) and (IIa):

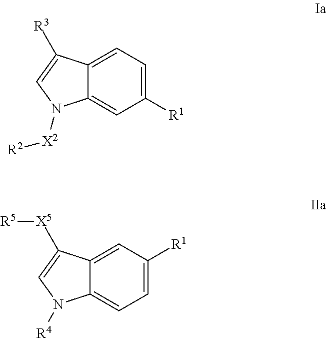

wherein:

### R¹ is —C(O)NHOH;

X² is a bond, alkylene, or alkenylene, where the alkylene or alkenylene is optionally substituted with one, two, three, four, or five halogens;
R² is aryl, cycloalkyl, heteroaryl, or heterocycloalkyl where the aryl, cycloalkyl, heteroaryl, and heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, or nitro;
R³ is hydrogen, alkyl, substituted alkyl, alkenyl, substituted alkenyl, hydroxy, alkoxy, or haloalkoxy;
R⁴ is hydrogen, alkyl, substituted alkyl, alkenyl, substituted alkenyl, hydroxy, alkoxy, haloalkoxy, or optionally substituted phenyl;
X⁵ is a bond, alkylene, or alkenylene where the alkylene or alkenylene is optionally substituted with halogen; and
R⁵ is aryl, cycloalkyl, heteroaryl, or heterocycloalkyl where the aryl, cycloalkyl, heteroaryl, and heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, or nitro; or
   an active metabolite, pharmaceutically acceptable solvate, pharmaceutically acceptable salt, pharmaceutically acceptable N-oxide, or pharmaceutically acceptable prodrug thereof.

In another embodiment, provided herein is a compound having a structure selected from among Formula Ib or IIb:

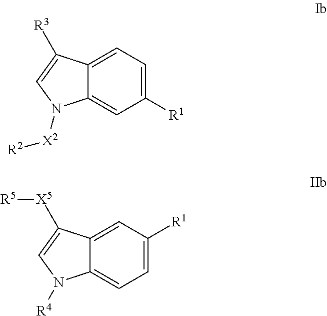

wherein:
R¹ is —C(O)NHOH;
X² is a bond, alkylene, or alkenylene where the alkylene or alkenylene is optionally substituted with one, two, three, four, or five halogens;
R² is aryl, cycloalkyl, heteroaryl, or heterocycloalkyl where the aryl is substituted with one, two, or three acyloxy, alkyl, substituted alkyl, alkenyl, substituted alkenyl, alkylamino, dialkylamino, or haloalkoxy; where the cycloalkyl is optionally substituted with one, two, or three acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, or nitro; and where the heteroaryl and the heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, carboxy, cyano, haloalkoxy, or nitro;
R³ is hydrogen, alkenyl, substituted alkenyl, hydroxy, alkoxy, haloalkoxy, or —X⁶—R⁶ where X⁶ is alkylene or alkenylene and X⁶ is additionally optionally substituted with one, two, three, four, of five halogens; and R⁶ is alkylcarbonyl, alkenylcarbonyl, optionally substituted cycloalkylcarbonyl, alkylcarbonyloxy, alkenylcarbonyloxy, amino, alkylamino, dialkylamino, cyano, cyanoalkylaminocarbonyl, alkoxy, alkenyloxy, hydroxyalkoxy, halogen, alkylcarbonylamino, alkyl-S(O)₀₋₂—, alkenyl-S(O)₀₋₂—, aminosulfonyl, alkylaminosulfonyl, dialkylaminosulfonyl, alkylsulfonyl-NR^{c}— (where R^{c} is hydrogen, alkyl, substituted alkyl, alkenyl, substituted alkynyl, hydroxy, alkoxy, or alkenyloxy), alkylaminocarbonyloxy, dialkylaminocarbonyloxy, alkylaminoalkyloxy, dialkylaminoalkyloxy, alkoxycarbonylamino, alkylaminocarbonylamino, dialkylaminocarbonylamino, alkoxyalkyloxy, or —C(O)NR^{a}R^{b} (where R^{a} and R^{b} are independently hydrogen, alkyl, substituted alkyl, alkenyl, alkynyl, substituted alkynyl, hydroxy, alkoxy, or alkenyloxy);
R⁴ is hydrogen, alkyl, substituted alkyl, alkenyl, substituted alkenyl, hydroxy, alkoxy, haloalkoxy, or optionally substituted phenyl; and
X⁵ is a bond; and R⁵ is phenyl, 3- to 8-membered monocyclic cycloalkyl, 5- or 6-membered monocyclic heteroaryl, or 3- to 8-membered monocyclic heterocycloalkyl where the 3- to 8-membered monocyclic cycloalkyl, 5- or 6-membered monocyclic heteroaryl, and 3- to 8-membered monocyclic heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, or nitro; and the phenyl is substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, or nitro; provided that R⁵ is not optionally substituted pyrrole or optionally substituted 2,5-dioxo-pyrrole; or
X⁵ is alkylene or alkenylene where the alkylene or alkenylene is optionally substituted with halogen; and R⁵ is aryl, cycloalkyl, heteroaryl, or heterocycloalkyl where the cycloalkyl, heteroaryl, and heterocycloalkyl are optionally substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, or nitro; and the aryl is substituted with one, two, or three acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, or nitro; or
   an active metabolite, pharmaceutically acceptable solvate, pharmaceutically acceptable salt, pharmaceutically acceptable N-oxide, or pharmaceutically acceptable prodrug thereof.

In one embodiment, provided herein is a compound of Formula (Ia).

In another embodiment, provided herein is a compound of Formula (Ib).

In yet another embodiment, provided herein is a compound of Formula (IIa).

In a further embodiment, provided herein is a compound of Formula (IIb).

In some embodiments, substituents are selected from among from a subset described herein. For example, in some embodiments, X² is a bond, alkylene, or alkenylene where the alkylene or alkenylene is optionally substituted with one, two, three, four, or five halogens. In another embodiment, X² is alkylene or alkenylene. In other embodiments, X² is —CH₂—, —CH₂CH₂—, —CH(CH₃)—, —(CH₂)₃—, or —CH₂CH═CH—. In some embodiments, X² is —CH₂—.

In some embodiments, R² is aryl, cycloalkyl, heteroaryl, or heterocycloalkyl where the aryl, cycloalkyl, heteroaryl, and heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro. In other embodiments, R² is aryl, cycloalkyl, heteroaryl, or heterocycloalkyl, where the aryl, cycloalkyl, heteroaryl, and heterocycloalkyl are optionally substituted with one, two, or three substituents selected from among alkyl, alkoxy, alkoxycarbonyl, halogen, and haloalkoxy. In some other embodiments, R² is aryl, cycloalkyl, heteroaryl, or heterocycloalkyl, where the aryl is optionally substituted with one, two, or three substituents selected from among alkyl, alkoxy, halogen, and haloalkoxy, and the heterocycloalkyl is optionally substituted with alkoxycarbonyl. In further embodiments, R² is cyclohexyl, benzooxadiazolyl, naphth-2-yl, phenyl, or piperidinyl, where the phenyl is optionally substituted with one, two, or three substituents selected from among methyl, methoxy, chloro, fluoro, trifluoromethoxy, and difluoromethoxy, and the piperidinyl is optionally substituted with t-butoxycarbonyl. In yet other embodiments, R² is cyclohexyl, benzo[2,1,3]oxadiazol-5-yl, phenyl, naphth-2-yl, 2-methylphenyl, 3-methylphenyl, 4-methylphenyl, 3-methoxyphenyl, 4-methoxyphenyl, 3,5-dimethoxyphenyl, 3,4,5-trimethoxyphenyl, 2-chlorophenyl, 3-chlorophenyl, 4-chlorophenyl, 3,4-dichlorophenyl, 2-fluorophenyl, 3-fluorophenyl, 4-fluorophenyl, 4-(difluoromethoxy)-phenyl, 4-(trifluoromethoxy)-phenyl, 3-fluoro-4-methoxy-phenyl, piperidin-4-yl, or N-(t-butoxycarbonyl)piperidin-4-yl.

In some embodiments, R² is benzo[2,1,3]oxadiazol-5-yl, 4-methoxyphenyl, 4-chlorophenyl, 4-(difluoromethoxy)-phenyl, or 3-fluoro-4-methoxy-phenyl.

In some embodiments, R³ is hydrogen, alkyl, substituted alkyl, alkenyl, substituted alkenyl, hydroxy, alkoxy, or haloalkoxy. In other embodiments, R³ is hydrogen.

In some embodiments, R⁴ is hydrogen, alkyl, substituted alkyl, alkenyl, substituted alkenyl, hydroxy, alkoxy, haloalkoxy, or optionally substituted phenyl. In yet other embodiments, R⁴ is alkyl or optionally substituted phenyl. In some other embodiments, R⁴ is methyl, ethyl, isopropyl, or phenyl. In some embodiments, R⁴ is methyl, ethyl, or isopropyl.

In some embodiments, X⁵ is a bond, alkylene, or alkenylene where the alkylene or alkenylene is optionally substituted with halogen. In other embodiments, X⁵ is alkylene. In yet other embodiments, X⁵ is —CH₂—.

In some embodiments, R⁵ is aryl, cycloalkyl, heteroaryl, or heterocycloalkyl where the aryl, cycloalkyl, heteroaryl, and heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro.

In yet other embodiments, R⁵ is heterocycloalkyl optionally substituted with alkoxycarbonyl or R⁵ is aryl optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro. In some embodiments, R⁵ is piperazinyl optionally substituted with t-butoxycarbonyl, or R⁵ is phenyl optionally substituted with one, two, or three substituents selected from among acylamino, amino, halogen, and nitro. In some other embodiments, R⁵ is 4-(t-butoxycarbonyl)piperazin-1-yl, phenyl, 4-aminophenyl, 4-(phenylcarbonylamino)-phenyl, 4-fluorophenyl, or 4-nitrophenyl. In yet other embodiments, R⁵ is phenyl, 4-aminophenyl, 4-(phenylcarbonylamino)-phenyl, 4-fluorophenyl, or 4-nitrophenyl.

In some embodiments, R² is aryl, cycloalkyl, heteroaryl, or heterocycloalkyl, where the aryl is substituted with one, two, or three substituents selected from among acyloxy, alkyl, substituted alkyl, alkenyl, substituted alkenyl, alkylamino, dialkylamino, and haloalkoxy; where the cycloalkyl is optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro; and where the heteroaryl and the heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, haloalkoxy, and nitro. In other embodiments, R² is aryl, cycloalkyl, heteroaryl, or heterocycloalkyl where the aryl is substituted with one, two, or three substituents selected from among alkyl and haloalkoxy, and the heterocycloalkyl is optionally substituted with alkoxycarbonyl. In yet other embodiments, R² is cyclohexyl; benzooxadiazolyl; phenyl substituted with one, two, or three substituents selected from among methyl, trifluoromethoxy, or difluoromethoxy; or piperidinyl optionally substituted with t-butoxycarbonyl.

In some embodiments, R² is cyclohexyl, benzo[2,1,3]oxadiazol-5-yl, 2-methylphenyl, 3-methylphenyl, 4-methylphenyl, 4-(difluoromethoxy)-phenyl, 4-(trifluoromethoxy)-phenyl, N-(t-butoxycarbonyl)piperidin-4-yl, or piperidin-4-yl. In yet other embodiments, R² is benzo[2,1,3]oxadiazol-5-yl or 4-(difluoromethoxy)-phenyl.

In some embodiments, R³ is hydrogen, alkenyl, substituted alkenyl, hydroxy, alkoxy, haloalkoxy, or —X⁶—R⁶, where X⁶ is alkylene or alkenylene and X⁶ is additionally optionally substituted with one, two, three, four, or five halogens; and R⁶ is alkylcarbonyl, alkenylcarbonyl, optionally substituted cycloalkylcarbonyl, alkylcarbonyloxy, alkenylcarbonyloxy, amino, alkylamino, dialkylamino, cyano, cyanoalkylaminocarbonyl, alkoxy, alkenyloxy, hydroxyalkoxy, halogen, alkylcarbonylamino, alkylcarbonyloxy, alkyl-S(O)₀₋₂—, alkenyl-S(O)₀₋₂—, aminosulfonyl, alkylaminosulfonyl, dialkylaminosulfonyl, alkylsulfonyl-NR^{c}— (where R^{c} is hydrogen, alkyl, substituted alkyl, alkenyl, substituted alkynyl, hydroxy, alkoxy, or alkenyloxy), alkylaminocarbonyloxy, dialkylaminocarbonyloxy, alkylaminoalkyloxy, dialkylaminoalkyloxy, alkoxycarbonylamino, alkylaminocarbonylamino, dialkylaminocarbonylamino, alkoxyalkyloxy, or —C(O)NR^{a}R^{b} (where R^{a} and R^{b} are independently hydrogen, alkyl, substituted alkyl, alkenyl, alkynyl, substituted alkynyl, hydroxy, alkoxy, or alkenyloxy). In some embodiments, R³ is hydrogen.

In some embodiments, X⁵ is a bond; and R⁵ is phenyl, 3- to 8-membered monocyclic cycloalkyl, 5- or 6-membered monocyclic heteroaryl, or 3- to 8-membered monocyclic heterocycloalkyl where the 3- to 8-membered monocyclic cycloalkyl, 5- or 6-membered monocyclic heteroaryl, and 3- to 8-membered monocyclic heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, or nitro; and the phenyl is substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro; provided that R⁵ is not optionally substituted pyrrole or optionally substituted 2,5-dioxo-pyrrole; or X⁵ is alkylene or alkenylene where the alkylene or alkenylene is optionally substituted with halogen; and R⁵ is aryl, cycloalkyl, heteroaryl, or heterocycloalkyl where the cycloalkyl, heteroaryl, and heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro; and the aryl is substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro.

In some embodiments, X⁵ is alkylene or alkenylene; and R⁵ is aryl substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro. In other embodiments, R⁵ is phenyl substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro. In some other embodiments, R⁵ is phenyl substituted with one, two, or three substituents selected from among optionally substituted arylcarbonylamino, amino, halogen, and nitro. In yet other embodiments, R⁵ is 4-(phenylcarbonylamino)-phenyl, 4-aminophenyl, 4-fluorophenyl, or 4-nitrophenyl.

In some embodiments, R³ is hydrogen; X² is alkylene or alkenylene; and R² is aryl, cycloalkyl, or heteroaryl, where the aryl, cycloalkyl, and heteroaryl are optionally substituted with one, two, or three substituents selected from among alkyl, alkoxy, alkoxycarbonyl, halogen, and haloalkoxy. In other embodiments, R³ is hydrogen; X² is alkylene or alkenylene; and R² is naphthyl, phenyl, cycloalkyl, heteroaryl, or heterocycloalkyl optionally substituted with methyl, methoxy, t-butoxycarbonyl, chloro, fluoro, trifluoromethoxy, or difluoromethoxy. In some other embodiments, R³ is hydrogen; X² is alkylene or alkenylene; and R² is phenyl where the phenyl is optionally substituted with one, two, or three substituents selected from among methyl, methoxy, chloro, fluoro, trifluoromethoxy, and difluoromethoxy; or R² is benzooxadiazolyl.

In some embodiments, R⁴ is hydrogen or alkyl; X⁵ is alkylene or alkenylene; and R⁵ is aryl optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro; or R⁵ is heterocycloalkyl optionally substituted with alkoxycarbonyl. In other embodiments, R⁴ is alkyl; X⁵ is alkylene; and R⁵ is phenyl optionally substituted with one, two, or three substituents selected from among acylamino, amino, halogen, and nitro.

In some embodiments, R³ is hydrogen; X² is alkylene or alkenylene; and R² is cycloalkyl, aryl, heteroaryl, or heterocycloalkyl, where the cycloalkyl is optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro; where the aryl is substituted with one, two, or three substituents selected from among acyloxy, alkyl, substituted alkyl, alkenyl, substituted alkenyl, alkylamino, dialkylamino, and haloalkoxy; where the heteroaryl and heterocycloalkyl are optionally substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, haloalkoxy, and nitro.

In some embodiments, R³ is hydrogen; X² is alkylene or alkenylene; and R² is cycloalkyl; phenyl substituted with one, two, or three alkyl or haloalkoxy; benzooxadiazolyl; or piperidinyl optionally substituted with alkoxycarbonyl. In some other embodiments, R³ is hydrogen; X² is alkylene or alkenylene; and R² is benzooxadiazolyl or phenyl where the phenyl is substituted with one, two, or three substituents selected from among methyl, chloro, fluoro, trifluoromethoxy, or difluoromethoxy.

In some embodiments, R⁴ is hydrogen or alkyl; X⁵ is a bond and R⁵ is heterocycloalkyl optionally substituted with alkoxycarbonyl; or X⁵ is alkylene or alkenylene and R⁵ is aryl substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, alkylaminocarbonyl, dialkylaminocarbonyl, optionally substituted arylaminocarbonyl, optionally substituted heteroarylaminocarbonyl, carboxy, cyano, halogen, haloalkoxy, and nitro.

In yet other embodiments, R⁴ is hydrogen or alkyl; X⁵ is alkylene; and R⁵ is phenyl substituted with one, two, or three substituents selected from among acyl, acylamino, acyloxy, alkyl, substituted alkyl, alkenyl, substituted alkenyl, alkoxy, alkoxycarbonyl, amino, alkylamino, dialkylamino, carboxy, cyano, halogen, haloalkoxy, and nitro. In yet other embodiments, R⁴ is hydrogen or alkyl; X⁵ is alkylene; and R⁵ is phenyl substituted with one, two, or three substituents selected from among optionally substituted arylcarbonylamino, amino, halogen, and nitro.

Any combination of the groups described above for the various variables is contemplated herein.

Further embodiments of compounds described herein (e.g. 1,3-disubstituted-1H-indole-6-carboxylic acid hydroxyamide compounds, 1,3-disubstituted-1H-indole-5-carboxylic acid hydroxyamide compounds, compounds of Formula (I), Formula (Ia), Formula (Ib), Formula (IIa), Formula (IIb)) include, but are not limited to, compounds in Tables 1 and 2.

TABLE 1
1,3-substituted-1H-indole-6-carboxylic acid hydroxyamides.

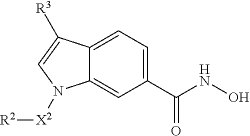

| Com- | | | |
|---|---|---|---|
| pound | | | |
| No. | R² | R³ | X² |
| 1. | 3,4-dichlorophenyl | H | —CH₂— |
| 2. | 2-methylphenyl | H | —CH₂— |
| 3. | 3,4,5- | H | —CH₂— |
| | trimethoxyphenyl | | |
| 4. | 3-fluorophenyl | H | —CH₂— |
| 5. | 3-methylphenyl | H | —CH₂— |
| 6. | phenyl | H | —CH₂— |
| 7. | 3,5-dimethoxyphenyl | H | —CH₂— |
| 8. | phenyl | H | —CH(CH₃)— |
| 9. | 4-fluorophenyl | H | —CH₂— |
| 10. | 2-fluorophenyl | H | —CH₂— |
| 11. | 2-chlorophenyl | H | —CH₂— |
| 12. | 3-methoxyphenyl | H | —CH₂— |
| 13. | naphth-2-yl | H | —CH₂— |
| 14. | phenyl | H | —(CH₂)₃— |
| 15. | cyclohexyl | H | —CH₂— |
| 16. | phenyl | H | —CH═CHCH₂— |
| 17. | 4-(trifluoromethoxy)- | H | —CH₂— |
| | phenyl | | |
| 18. | 4-chlorophenyl | H | —CH₂— |
| 19. | benzo[2,1,3]oxadiazol- | H | —CH₂— |
| | 5-yl | | |
| 20. | 4-methylphenyl | H | —CH₂— |
| 21. | 3-fluoro-4-methoxy- | H | —CH₂— |
| | phenyl | | |
| 22. | 4-(difluoromethoxy)- | H | —CH₂— |
| | phenyl | | |
| 23. | 4-methoxyphenyl | H | —CH₂— |
| 24. | phenyl | H | —CH₂CH₂— |
| 25. | 3-chlorophenyl | H | —CH₂— |
| 26. | N-(t-butoxycarbonyl) | H | —CH₂— |
| | piperidin-4-yl | | |
| 27. | piperidin-4-yl | H | —CH₂— |
| 28. | N-methylsulfonyl-3- | H | —CH₂— |
| | aminophenyl | | |
| 29. | 4-methoxyphenyl | dimethylaminomethyl | —CH₂— |
| 30. | 4-methoxyphenyl | N-morpholinomethyl | —CH₂— |
| 31. | 4-methoxyphenyl | N-pyrrolidinomethyl | —CH₂— |
| 32. | 4-methoxyphenyl | N-benzylaminomethyl | —CH₂— |
| 33. | 4-methoxyphenyl | ethyl | —CH₂— |

Compounds in Table 1 are named:
1-(3,4-dichloro-phenylmethyl)-1H-indole-6-carboxylic acid hydroxyamide (Compound 1);
1-(2-methyl-phenylmethyl)-1H-indole-6-carboxylic acid hydroxyamide (Compound 2);
1-(3,4,5-trimethoxy-phenylmethyl)-1H-indole-6-carboxylic acid hydroxyamide (Compound 3);
1-(3-fluoro-phenylmethyl)-1H-indole-6-carboxylic acid hydroxyamide (Compound 4);
1-(3-methyl-phenylmethyl)-1H-indole-6-carboxylic acid hydroxyamide (Compound 5);
1-(benzyl)-1H-indole-6-carboxylic acid hydroxyamide (Compound 6);
1-(3,5-dimethoxy-phenylmethyl)-1H-indole-6-carboxylic acid hydroxyamide (Compound 7);
1-(1-methyl-1-phenylmethyl)-1H-indole-6-carboxylic acid hydroxyamide (Compound 8);
1-(4-fluoro-phenylmethyl)-1H-indole-6-carboxylic acid hydroxyamide (Compound 9);
1-(2-fluoro-phenylmethyl)-1H-indole-6-carboxylic acid hydroxyamide (Compound 10);
1-(2-chloro-phenylmethyl)-1H-indole-6-carboxylic acid hydroxyamide (Compound 11);
1-(3-methoxy-phenylmethyl)-1H-indole-6-carboxylic acid hydroxyamide (Compound 12);
1-(naphth-2-ylmethyl)-1H-indole-6-carboxylic acid hydroxyamide (Compound 13);
1-(3-phenylpropyl)-1H-indole-6-carboxylic acid hydroxyamide (Compound 14);
1-(cyclohexylmethyl)-1H-indole-6-carboxylic acid hydroxyamide (Compound 15);
1-[1-(phenyl)-propen-3-yl]-1H-indole-6-carboxylic acid hydroxyamide (Compound 16);
1-[4-(trifluoromethoxy)-phenylmethyl]-1H-indole-6-carboxylic acid hydroxyamide (Compound 17);
1-(4-chloro-phenylmethyl)-1H-indole-6-carboxylic acid hydroxyamide (Compound 18);
1-(benzo[2,1,3]oxadiazol-5-ylmethyl)-1H-indole-6-carboxylic acid hydroxyamide (Compound 19;
1-(4-methyl-phenylmethyl)-1H-indole-6-carboxylic acid hydroxyamide (Compound 20);
1-(3-fluoro-4-methoxy-phenylmethyl)-1H-indole-6-carboxylic acid hydroxyamide (Compound 21);
1-[4-(difluoromethoxy)-phenylmethyl]-1H-indole-6-carboxylic acid hydroxyamide (Compound 22);
1-(4-methoxy-phenylmethyl)-1H-indole-6-carboxylic acid hydroxyamide (Compound 23);
1-(phenethyl)-1H-indole-6-carboxylic acid hydroxyamide (Compound 24);
1-(3-chloro-phenylmethyl)-1H-indole-6-carboxylic acid hydroxyamide (Compound 25);
1-[N-(t-butoxycarbonyl)piperidin-4-ylmethyl]-1H-indole-6-carboxylic acid hydroxyamide (Compound 26);
1-(piperidin-4-ylmethyl)-1H-indole-6-carboxylic acid hydroxyamide (Compound 27);
1-(N-methylsulfonyl-3-aminobenzyl)-1H-indole-6-carboxylic acid hydroxyamide (Compound 28);
3-(Dimethylaminomethyl)-1-(4-methoxybenzyl)-1H-indole-6-carboxylic acid hydroxyamide (Compound 29);
3-(N-Morpholinomethyl)-1-(4-methoxybenzyl)-1H-indole-6-carboxylic acid hydroxyamide (Compound 30);
3-(N-Pyrrolidinomethyl)-1-(4-methoxybenzyl)-1H-indole-6-carboxylic acid hydroxyamide (Compound 31);
3-(N-Benzylaminomethyl)-1-(4-methoxybenzyl)-1H-indole-6-carboxylic acid hydroxyamide (Compound 32); and
3-(Ethyl)-1-(4-methoxybenzyl)-1H-indole-6-carboxylic acid hydroxyamide (Compound 33).

TABLE 2
1,3-substituted-1H-indole-5-carboxylic acid hydroxyamides

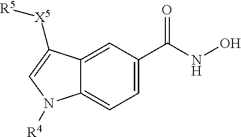

| Compound No. | R⁴ | X⁵ | R⁵ |
|---|---|---|---|
| 34. | methyl | —CH₂— | 4-nitrophenyl |
| 35. | ethyl | —CH₂— | phenyl |
| 36. | methyl | —CH₂— | 4-(phenylcarbonylamino)-phenyl |
| 37. | isopropyl | —CH₂— | phenyl |
| 38. | methyl | —CH₂— | 4-aminophenyl |
| 39. | methyl | —CH₂— | 4-fluorophenyl |
| 40. | phenyl | —CH₂— | phenyl |
| 41. | methyl | —CH₂— | 4-(t-butoxycarbonyl)piperazin-1-yl |

Compounds in Table 2. are named:
1-methyl-3-(4-nitro-phenylmethyl)-1H-indole-5-carboxylic acid hydroxyamide (Compound 34);
1-ethyl-3-(phenylmethyl)-1H-indole-5-carboxylic acid hydroxyamide (Compound 35);
1-methyl-3-[4-(phenylcarbonylamino)-phenylmethyl]-1H-indole-5-carboxylic acid hydroxyamide (Compound 36);
1-isopropyl-3-(phenylmethyl)-1H-indole-5-carboxylic acid hydroxyamide (Compound 37);
1-methyl-3-(4-amino-phenylmethyl)-1H-indole-5-carboxylic acid hydroxyamide (Compound 38);
1-methyl-3-(4-fluoro-phenylmethyl)-1H-indole-5-carboxylic acid hydroxyamide (Compound 39);
1-phenyl-3-(phenylmethyl)-1H-indole-5-carboxylic acid hydroxyamide (Compound 40); and
1-methyl-3-[4-(t-butoxycarbonyl)piperazin-1-ylmethyl]-1H-indole-5-carboxylic acid hydroxyamide (Compound 41).

### Further Forms of Compounds

For compounds described herein that possess one or more stereocenters, each center exists in the R or S configuration. The compounds presented herein include all diastereomeric, enantiomeric, and epimeric forms as well as the appropriate mixtures thereof. In one embodiment, separation of stereoisomers is performed by chromatography. In some embodiments, individual stereoisomers are obtained by reacting a racemic mixture of the compound with an optically active resolving agent to form a pair of diastereoisomeric compounds, separating the diastereomers and recovering the optically pure enantiomers. In other embodiments, while resolution of enantiomers are carried out using covalent diastereomeric derivatives of the compounds described herein, dissociable complexes are contemplated herein (e.g., crystalline diastereomeric salts). Diastereomers have distinct physical properties (e.g., melting points, boiling points, solubilities, reactivity, etc.) and are readily separated by taking advantage of these dissimilarities. In some embodiments, the diastereomers are separated by chiral chromatography, or by separation/resolution techniques based upon differences in solubility. The optically pure enantiomer(s) is/are then recovered, along with the resolving agent, by any practical means that would not result in racemization. A more detailed description of the techniques applicable to the resolution of stereoisomers of compounds from their racemic mixture are found in Jean Jacques, Andre Collet, Samuel H. Wilen, “Enantiomers, Racemates and Resolutions”, John Wiley And Sons, Inc., 1981, herein incorporated by reference in its entirety. In further embodiments, stereoisomers are also be obtained by stereoselective synthesis.

For compounds described herein that exist as tautomers, all tautomers are included within the formulas described herein.

The methods and formulations described herein include the use of N-oxides, crystalline forms (also known as polymorphs), or pharmaceutically acceptable salts of compounds described herein, as well as active metabolites of these compounds having the same type of activity. In addition, the compounds described herein exist in unsolvated as well as solvated forms with pharmaceutically acceptable solvents such as water, ethanol, and the like. The solvated forms of the compounds presented herein are also considered to be disclosed herein.

In some embodiments, indole compounds described herein in unoxidized form are prepared from the corresponding N-oxides indole compounds by treating with a reducing agent, such as, but not limited to, sulfur, sulfur dioxide, triphenyl phosphine, lithium borohydride, sodium borohydride, phosphorus trichloride, phosphorus tribromide, or the like in a suitable inert organic solvent, such as, but not limited to, acetonitrile, ethanol, aqueous dioxane, or the like at 0 to 80° C.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. In one embodiment, the prodrug has improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. In a further embodiment, is a prodrug having a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a prodrug, a pharmaceutically active compound is modified such that the active compound is regenerated upon in vivo administration. In some embodiments, the prodrug is designed to alter the metabolic stability or the transport characteristics of a drug, to mask side effects or toxicity, to improve the flavor of a drug or to alter other characteristics or properties of a drug. Knowledge of pharmacodynamic processes and drug metabolism in vivo permits design prodrugs of the compound. (see, for example, Nogrady (1985) Medicinal Chemistry A Biochemical Approach, Oxford University Press, New York, pages 388-392; Silverman (1992).

Prodrug forms of the herein described compounds, wherein the prodrug is metabolized in vivo to produce a derivative as set forth herein are included within the scope of the claims. In some cases, some of the herein-described compounds are a prodrug for another derivative or active compound.

In some embodiments, are prodrugs which are designed as reversible drug derivatives, for use as modifiers to enhance drug transport to site-specific tissues. In some embodiments, the design of a prodrug increases the effective water solubility.

In other embodiments, sites on the aromatic ring portion of compounds described herein are susceptible to various metabolic reactions, therefore incorporation of appropriate substituents on the aromatic ring structures, such as, by way of example only, halogens which reduce, minimize or eliminate this metabolic pathway.

In one embodiment, the compounds described herein are labeled isotopically (e.g. with a radioisotope) or by other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. In some embodiments, isotopes that are incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, in other embodiments, substitution with isotopes such as deuterium, i.e., ²H, affords certain therapeutic advantages resulting from greater metabolic stability, for example increased in vivo half-life or reduced dosage requirements.

In additional or further embodiments, the compounds described herein are metabolized upon administration to an organism in need to produce a metabolite that is then used to produce a desired effect, including a desired therapeutic effect.

In one embodiment, compounds described herein are formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, nitric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion. In some cases, compounds described herein coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. In one embodiment, are solvates which contain either stoichiometric or non-stoichiometric amounts of a solvent, and are formed during the process of crystallization with pharmaceutically acceptable solvents such as water, ethanol, and the like. In other embodiments, are hydrates formed when the solvent is water. In yet other embodiments, are alcoholates formed when the solvent is alcohol. In a further embodiment, are solvates of compounds described herein conveniently prepared or formed during the processes described herein. In other embodiments, the compounds provided herein exist in unsolvated as well as solvated forms. In general, the solvated forms are considered equivalent to the unsolvated forms for the purposes of the compounds and methods provided herein.

In other embodiments are compounds, described herein, in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature cause a single crystal form to dominate.

In some embodiments, the screening and characterization of the pharmaceutically acceptable salts, polymorphs and/or solvates are accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UV-VIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

### Synthesis of Compounds

In some embodiments, the synthesis of compounds described herein are accomplished using means described in the chemical literature, using the methods described herein, or by a combination thereof.

In other embodiments, the starting materials and reagents used for the synthesis of the compounds described herein are synthesized or are obtained from commercial sources, such as, but not limited to, Aldrich Chemical Co. (Milwaukee, Wis.), Sigma Chemical Co. (St. Louis, Mo.), or Bachem (Torrance, Calif.).

In some embodiments, the compounds described herein, and other related compounds having different substituents are synthesized using techniques and materials described herein. In some embodiments, the following synthetic methods are utilized.

In other embodiments, indole compounds described herein are synthesized starting from indole compounds that are available from commercial sources or they are prepared using procedures outlined herein.

Using the reaction conditions described herein, 1,3-substituted-1H-indole-5-carboxylic acid hydroxyamides and 1,3-substituted-1H-indole-6-carboxylic acid hydroxyamides as disclosed herein are obtained in good yields and purity. The compounds prepared by the methods disclosed herein are purified by methods including, for example, filtration, recrystallization, chromatography, distillation, and combinations thereof.

Schemes presented herein are merely illustrative of some methods by which the compounds described herein are synthesized, and various modifications to these schemes are contemplated herein.

Formation of Covalent Linkages by Reaction of an Electrophile with a Nucleophile

The compounds described herein can be modified using various electrophiles and/or nucleophiles to form new functional groups or substituents. Table 3 entitled “Examples of Covalent Linkages and Precursors Thereof” lists selected non-limiting examples of covalent linkages and precursor functional groups which yield the covalent linkages. Table 3 may be used as guidance toward the variety of electrophiles and nucleophiles combinations available that provide covalent linkages. Precursor functional groups are shown as electrophilic groups and nucleophilic groups.

TABLE 3
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it may be necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. It is preferred that each protective group be removable by a different means. Protective groups that are cleaved under totally disparate reaction conditions fulfill the requirement of differential removal.

Protective groups can be removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, which include conversion to alkyl esters, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference in their entirety.

### General Synthesis

In some embodiments, indole compounds described herein are prepared from commercially available materials or they are prepared by suitable methods.

In one embodiment, compounds of structure 1 and structure 2 are used as starting materials for the synthesis of compounds described herein.

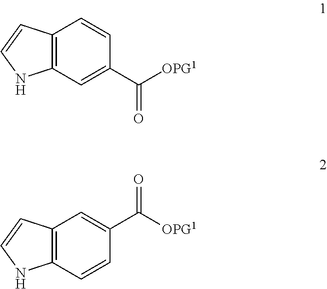

PG¹ represents carboxylic acid protecting groups. In one embodiment, PG¹ represents a substituted or unsubstituted alkyl group, such as, but not limited to, methyl, ethyl, propyl, benzyl, and p-methoxybenzyl.

In other embodiments, indoles of general structure 1 and structure 2 are also prepared by suitable methods. Indole containing compounds described herein are prepared using standard literature procedures such as those found in Katritzky, “Handbook of Heterocyclic Chemistry” Pergamon Press, Oxford, 1986; Pindur et al, J. Heterocyclic Chem., vol 25, 1, 1987, and Robinson “The Fisher Indole Synthesis”, John Wiley & Sons, Chichester, N.Y., 1982, each of which is herein incorporated by reference in their entirety.

Additional non-limiting examples of synthetic strategies toward the synthesis of indole compounds described herein, include modifications to various syntheses of indoles, including, but not limited to: Batcho-Leimgruber Indole Synthesis, Reissert Indole Synthesis, Hegedus Indole Synthesis, Fukuyama Indole Synthesis, Sugasawa Indole Synthesis, Bischler Indole Synthesis, Gassman Indole Synthesis, Fischer Indole Synthesis, Japp-Klingemann Indole Synthesis, Buchwald Indole Synthesis, Larock Indole Synthesis, Bartoli Indole Synthesis, Castro Indole Synthesis, Hemetsberger Indole Synthesis, Mori-Ban Indole Synthesis, Madelung Indole Synthesis, Nenitzescu Indole Synthesis, and other unnamed reactions.

In one embodiment, the functionalization of the 1-position and/or 3-position of indoles of structure 1 and structure 2 is achieved by using any of the indole forming reactions mentioned above with appropriate starting materials.

In another embodiment, the 1-position of indoles described herein is functionalized as outlined in Scheme 1.

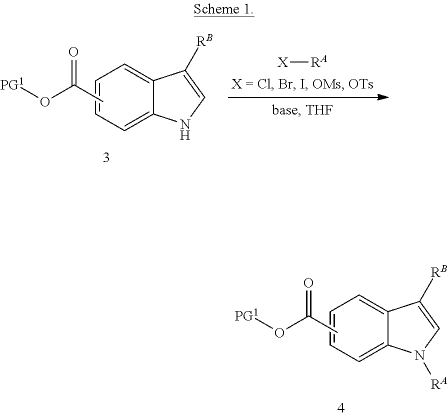

Indoles of general structure 4 (where R^{B} is H, R³, or —X⁵—R⁵; R^{A} is R⁴ or —X²—R²) are obtained from the N-alkylation of indoles of structure 3 with, for example, an alkyl halide (or benzyl halide, or tosylate (OTs) or mesylate (OMs), or carboxylic acid halide) in a solvent such as tetrahydrofuran (THF) or dimethylformamide (DMF) in the presence of a base, such as, for example, NaH or potassium carbonate or sodium carbonate. In other embodiments, N-arylation of indoles is achieved using a metal mediated cross coupling of N—H indoles of general structure 3 with aryl halides or triflates (R^{A} is aryl, heteroaryl; Old et al. Org. Lett., 2 (10), 1403-1406, 2000.).

In addition, when R^{B} is a bromine or iodine, standard cross coupling reactions allow the introduction of a variety of functional groups using standard procedures. In other embodiments, indoles of structure 3, where R^{B} is a halide are prepared using standard bromination conditions or iodination conditions. Metal mediated coupling reactions include, but are not limited to Suzuki reactions, Sonogashira couplings, Heck reactions, Stille cross couplings, Negishi couplings, Kumada couplings, Ullmann reactions, Buchwald-Hartwig reactions, and variants thereof. (Metal-Catalyzed Cross-Coupling Reactions, Armin de Meijere (Editor), Francois Diederich (Editor), John Wiley & Sons; 2nd edition, 2004).

Other non-limiting approaches to the functionalization of indoles at the 1-position and/or 3-position are shown in Scheme 2.

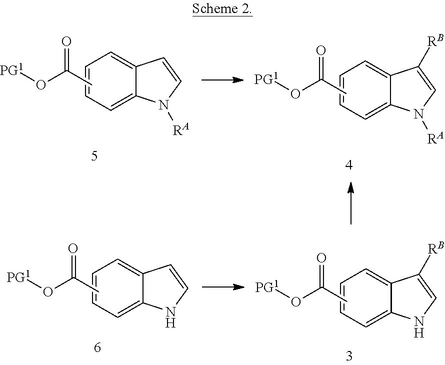

In other embodiments, functionalization at the 3-position of 3-H-indoles of structure 5 (R^{A} is R⁴ or —X²—R²) are achieved using a variety of reactions and procedures to allow the introduction of a wide range of substituents. By way of example only, acylation using an acid chloride (or anhydride) in the presence of a Lewis acid such as AlCl₃, allows for the introduction of acyl groups at the 3-position of indoles. Selective reduction of the carbonyl at the 3-position of the indole provides compounds of structure 4 (where R^{B} is R³, or —X⁵—R⁵, which is a substituted or unsubstituted alkyl; R^{A} is R⁴ or —X²—R²).

The reaction of electron deficient olefins with 3-H indoles of structure 5 (R^{A} is R⁴ or —X²—R²) or structure 6 in the presence of a Lewis acid (such as, for example, Yb(OTf)₃.3H₂O) allows the installation of alkyl substituents at the 3-position of the indole compounds to provide indoles of the general structure 4 or 3 (where R^{B} is R³, or —X⁵—R⁵, which is a substituted alkyl group. In other embodiments, indoles of structure 6 are reacted with benzyl derivatives in warm DMF to yield indoles of structure 3 where R^{B} is R³, or —X⁵—R⁵, which is a substituted benzyl group.

In other embodiments, indoles of general structure 5 or 6 are reacted with methyl ketones in the presence of a base and copper catalyst in order to provide indoles of general structure 3 or 4, where R^{B} is a substituted alkyl.

In other embodiments, compounds of general structure 5 are reacted with alkyl halides in the presence of a lewis acid, such as, silver oxide, to provide compounds of general structure 4.

As shown in Scheme 3,3-formyl indoles of general structure 7 are condensed with a variety of amines in the presence of a hydride source to provide substituted 3-aminoalkyls of general structure 8.

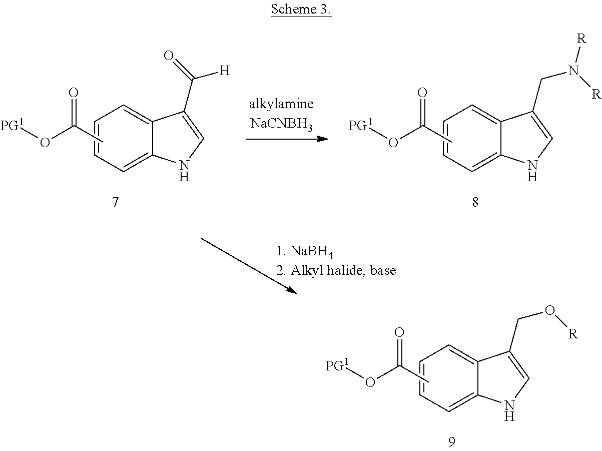

In other embodiments, 3-formyl indoles of general structure 7 are reduced to the alcohol by treatment with a mild hydride source, such as, but not limited to, sodium borohydride. The alcohol is coupled with a variety of electrophiles, such as, but not limited to, alkyl halides, carboxylic acid halides, to provide compounds of structure 9. 3-In further embodiments, formyl indoles of structure 7 are prepared using the Vilsmeir reaction or are commercially available.

Conversion of the indoles of general structure 4 (where R^{B} is R³ or —X⁵—R⁵; R^{A} is R⁴ or —X²—R²) to the corresponding 1,3-substituted-1H-indole-5-carboxylic acid hydroxyamides or 1,3-substituted-1H-indole-6-carboxylic acid hydroxyamides is shown in Scheme 4.

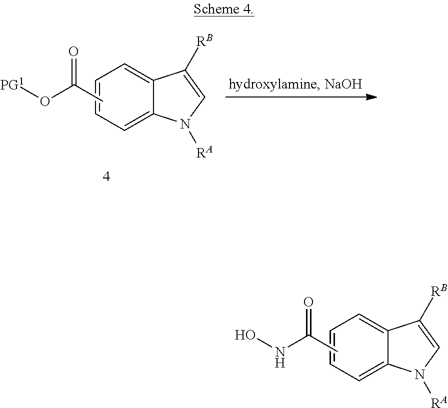

Indoles of structure 4 are treated with sodium hydroxide and an aqueous solution of hydroxylamine to provide the corresponding 1,3-substituted-1H-indole-5-carboxylic acid hydroxyamides or 1,3-substituted-1H-indole-6-carboxylic acid hydroxyamides. In embodiments where PG¹ is H in structure 4, the carboxylic acid is reacted with hydroxylamine hydrochloride salt using a coupling agent such as, but not limited to, 2-(7-Aza-1H-benzotriazole-1-yl)-1,1,3,3-tetramethyluronium hexafluorophosphate (HATU), dicyclohexyl carbodiimide (DCC), and the like, in the presence of a base such as, but not limited to, N,N-diisopropylethylamine, triethylamine, and the like, in a solvent such as, but not limited to, DMF, THF, and the like. In another embodiment, where PG¹ is H in structure 4, the carboxylic acid is reacted with thionyl chloride or oxalyl chloride to provide the acid chloride, which is treated with hydroxylamine to furnish the indole hydroxamic acid compounds.

In one embodiment, indole-6-hydroxamic acids described herein is synthesized by a process that includes:

(a) reacting an intermediate of Formula 1:

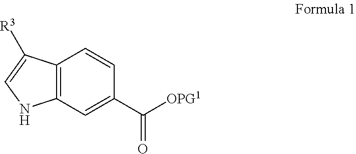

where PG¹ is a carboxy-protecting group, such as, but not limited to, methyl, ethyl, propyl, benzyl, p-methoxybenzyl, and the like;
with a compound having a formula R²—X²—Y, where Y is a halide, to yield an intermediate of Formula 6:

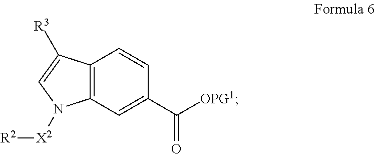

(b) optionally reducing the intermediate of Formula 6 where R² is phenyl substituted with nitro to yield an intermediate of formula 10:

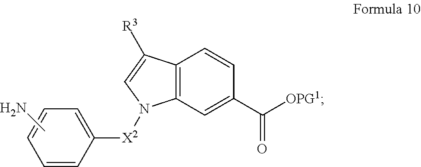

(c) optionally reacting the intermediate of Formula 6 where R⁵ is phenyl substituted with amino or alkylamino or reacting the intermediate of Formula 10 with ROH where R is acyl or alkylsulfonyl, as defined herein, to yield an intermediate of Formula 11:

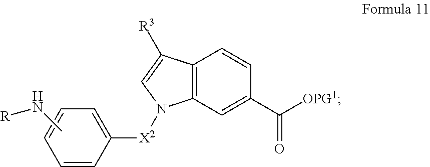

(d) optionally reacting the intermediate of Formula 6 where R⁵ is phenyl substituted with carboxy with NH₂(alkyl) or NH(alkyl)₂ to yield an intermediate of Formula 12:

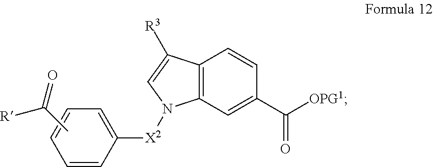

where R′ is alkylamino or dialkylamino;
(e) deprotecting the intermediate of Formula 6, the intermediate of Formula 10, the intermediate of Formula 11, and the intermediate of Formula 12 to yield a corresponding carboxylic acid;
(e) reacting the carboxylic acid from Step (e) with hydroxylamine to yield a indole-hydroxamic acid compound described herein; and
(f) optionally separating individual isomers.

In another embodiment, provided herein is a method of making indole 5-hydroxamic acids, which includes:

(a) reacting an intermediate of Formula 2:

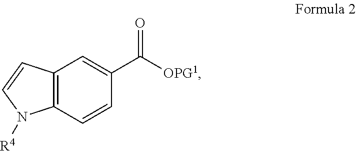

where PG¹ is a carboxy-protecting group,
with an intermediate of formula R⁵—X⁵—Y where Y is a halide to yield an intermediate of Formula 4:

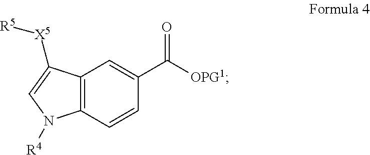

(b) optionally reducing the intermediate of Formula 4 where R⁵ is phenyl substituted with nitro to yield an intermediate of Formula 7:

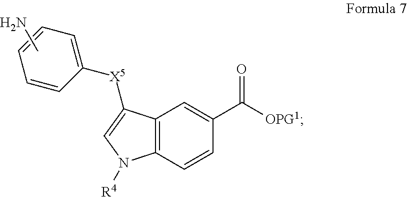

(c) optionally reacting the intermediate of formula 4 where R⁵ is phenyl substituted with amino or alkylamino or reacting the intermediate of Formula 7 with ROH where R is acyl, as defined herein, to yield an intermediate of Formula 8:

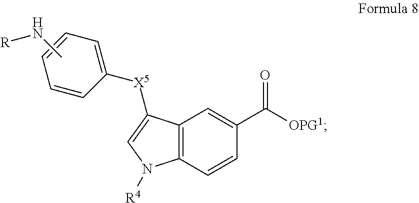

(d) optionally reacting the intermediate of formula 4 where R⁵ is phenyl substituted with carboxy with NH₂(alkyl) or NH(alkyl)₂ to yield an intermediate of Formula 9:

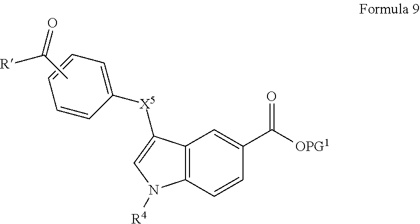

where R′ is alkylamino or dialkylamino;
(e) deprotecting the intermediate of formula 4, the intermediate of Formula 7, the intermediate of Formula 8, and the intermediate of Formula 9 to yield a corresponding carboxylic acid;
(f) reacting the carboxylic acid from Step (e) with hydroxylamine to yield a indole 5-hydroxamic acid described herein; and
(g) optionally separating individual isomers.

### CERTAIN TERMINOLOGY

Definition of standard chemistry terms are found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology are employed. In addition, nucleic acid and amino acid sequences for HDAC8 are disclosed in, e.g., U.S. Pat. No. 6,875,598. Unless specific definitions are provided, the nomenclature employed in connection with, and the laboratory procedures and techniques of, analytical chemistry, synthetic organic chemistry, and medicinal and pharmaceutical chemistry described herein are standard in organic chemistry. Standard techniques are used for chemical syntheses, chemical analyses, pharmaceutical preparation, formulation, and delivery, and treatment of patients. Standard techniques are used for recombinant DNA, oligonucleotide synthesis, and tissue culture and transformation (e.g., electroporation, lipofection). Reactions and purification techniques are performed e.g., using kits of manufacturer's specifications or as commonly accomplished or as described herein.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ. C₁-Cₓ refers to the number of carbon atoms that make up the moiety to which it designates (excluding optional substitutents).

An “alkyl” group refers to an aliphatic hydrocarbon group. In some embodiments, the alkyl moiety is a “saturated alkyl” group, which means that it does not contain any alkene or alkyne moieties. In other embodiments, the alkyl moiety is also an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. In other embodiments, the alkyl moiety, whether saturated or unsaturated, is branched, straight chain, or cyclic.

In a further embodiment, the “alkyl” moiety has 1 to 10 carbon atoms (whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated). In another embodiment, the alkyl group of the compounds described herein is designated as “C₁-C₆ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, t-butyl, pentyl, iso-pentyl, neo-pentyl, and hexyl. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. In further embodiments, alkyl groups are substituted or unsubstituted. In some embodiments, depending on the structure, an alkyl group is a monoradical or a diradical (i.e., an alkylene group).

An “alkoxy” group refers to a (alkyl)O— group, where alkyl is as defined herein. Examples of alkoxy groups include, but are not limited to, methoxy, ethoxy, propoxy, isopropoxy, butyloxy, cyclopropyloxy, cyclopentyloxy, cyclohexyloxy, and the like.

The term “alkenyl” refers to a type of alkyl group in which the first two atoms of the alkyl group form a double bond that is not part of an aromatic group. That is, in some embodiments, an alkenyl group begins with the atoms —C(R)═CR₂, wherein R refers to the remaining portions of the alkenyl group, which are the same or different. Non-limiting examples of an alkenyl group include —CH═CH₂, —C(CH₃)═CH₂, —CH═CHCH₃ and —C(CH₃)═CHCH₃. In other embodiments, the alkenyl moiety is branched, straight chain, or cyclic (in which case, it would also be known as a “cycloalkenyl” group). In other embodiments, alkenyl groups have 2 to 6 carbons. In further embodiments, alkenyl groups are substituted or unsubstituted. In another embodiment, depending on the structure, an alkenyl group is a monoradical or a diradical (i.e., an alkenylene group).

The term “alkynyl” refers to a type of alkyl group in which the first two atoms of the alkyl group form a triple bond. That is, an alkynyl group begins with the atoms —C≡C—R, wherein R refers to the remaining portions of the alkynyl group. Non-limiting examples of an alkynyl group include —C≡CH, —C≡CCH₃, —C≡CCH₂CH₃ and —C≡CCH₂CH₂CH₃. In some embodiments, the “R” portion of the alkynyl moiety is branched, straight chain, or cyclic. In other embodiments, an alkynyl group has 2 to 6 carbons. In further embodiments, alkynyl groups are substituted or unsubstituted. In further embodiments, depending on the structure, an alkynyl group is a monoradical or a diradical (i.e., an alkynylene group).

“Amino” refers to a —NH₂ group, or an N-oxide derivative.

The term “alkylamine” or “alkylamino” refers to the —N(alkyl)ₓH_{y} group, where alkyl is as defined herein and x and y are selected from the group x=1, y=1 and x=2, y=0. In other embodiments, when x=2, the alkyl groups, taken together with the nitrogen to which they are attached, optionally form a cyclic ring system. “Dialkylamino” refers to a —N(alkyl)₂ group, where alkyl is as defined herein.

An “amide” is a chemical moiety with formula —C(O)NHR or —NHC(O)R, where R is selected from the group consisting of alkyl, cycloalkyl, aryl, heteroaryl (bonded through a ring carbon) and heteroalicyclic (bonded through a ring carbon). In some embodiments, an amide is an amino acid or a peptide molecule attached to a compound, thereby forming a prodrug. In other embodiments, any amine, or carboxyl side chain on the compounds described herein are amidified. The procedures and specific groups to make such amides include those found in, e.g., Greene and Wuts, Protective Groups in Organic Synthesis, 3^{rd} Ed., John Wiley & Sons, New York, N.Y., 1999, which is incorporated herein by reference in its entirety.

The term “ester” refers to a chemical moiety with formula —C(═O)OR, where R is selected from the group consisting of alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters include those found in, e.g., Greene and Wuts, Protective Groups in Organic Synthesis, 3^{rd} Ed., John Wiley & Sons, New York, N.Y., 1999, which is incorporated herein by reference in its entirety.

As used herein, the term “aryl” refers to an aromatic ring wherein each of the atoms forming the ring is a carbon atom. In other embodiments, aryl rings are formed by five, six, seven, eight, nine, or more than nine carbon atoms. In other embodiments, aryl groups are optionally substituted. Examples of aryl groups include, but are not limited to phenyl, and naphthalenyl. In some embodiments, depending on the structure, an aryl group is a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. In some embodiments, cycloalkyls are saturated, or partially unsaturated. In other embodiments, cycloalkyls are fused with an aromatic ring. Cycloalkyl groups include groups having from 3 to 10 ring atoms. Illustrative examples of cycloalkyl groups include, but are not limited to, the following moieties:

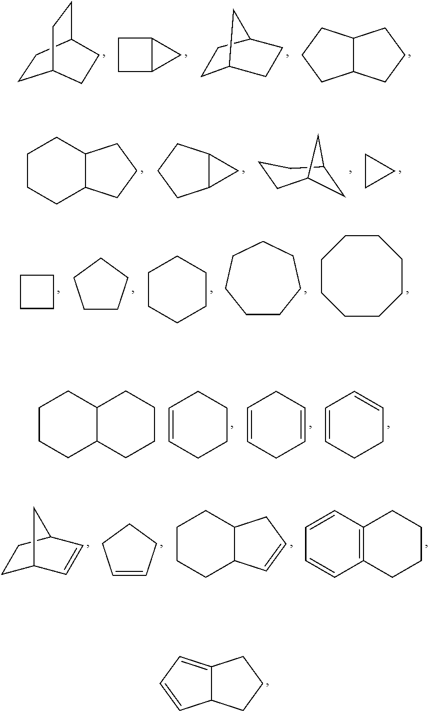

and the like. Monocyclic cycloalkyls include, but are not limited to, cyclopropyl, cyclobutyl, cyclopentyl, cyclohexyl, cycloheptyl, and cyclooctyl.

“Cycloalkylalkyl” refers to an alkyl, as is defined herein, substituted with a cycloalkyl, as is defined herein.

The term “heterocycle” refers to heteroaromatic and heteroalicyclic groups containing one to four ring heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups may be C-attached or N-attached where such is possible. For example, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In some embodiments, polycyclic heteroaryl groups are fused or non-fused. Illustrative examples of heteroaryl groups include the following moieties:

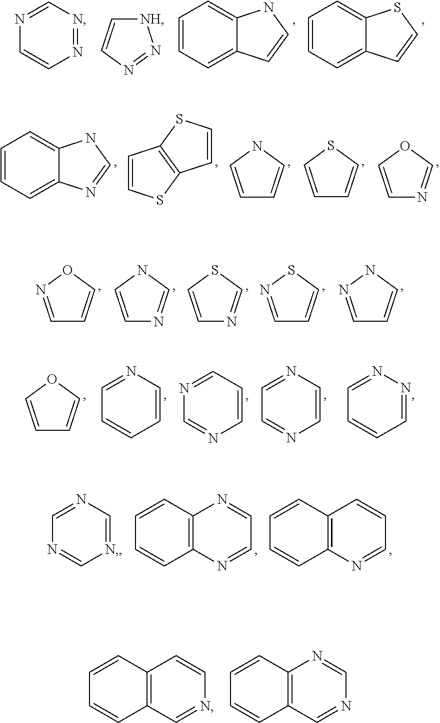

and the like.

In some embodiments, substituted or unsubstituted heteroaryl groups are selected from among pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, 4-azaindolyl, 5-azaindolyl, 6-azaindolyl, 7-azaindolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, imidazo[1,2-a]pyridinyl, thiophenopyridinyl, and furopyridinyl. In other embodiments, substituted or unsubstituted heteroaryl groups is selected from among pyridinyl, pyrimidinyl, pyrazinyl, quinolinyl, isoquinolinyl, indolyl, benzimidazolyl, benzofuranyl, cinnolinyl, indazolyl, indolizinyl, phthalazinyl, pyridazinyl, isoindolyl, pteridinyl, purinyl, oxadiazolyl, thiadiazolyl, furazanyl, benzofurazanyl, benzothienyl, benzothiazolyl, benzoxazolyl, quinazolinyl, quinoxalinyl, naphthyridinyl, imidazo[1,2-a]pyridinyl, thiophenopyridinyl, and furopyridinyl. In yet other embodiments, substituted or unsubstituted heteroaryl groups are selected from among pyridinyl, pyrimidinyl, pyrazinyl, quinolinyl, isoquinolinyl, pyridazinyl, quinazolinyl, quinoxalinyl. In still other embodiments, substituted or unsubstituted heteroaryl groups are selected from among pyridinyl, and quinolinyl.

“Heteroaralkyl” or “heteroarylalkyl” refers to an alkyl, as is defined herein, substituted with a heteroaryl as is defined herein.

A “heteroalicyclic” group or “heterocycloalkyl” group refers to a cycloalkyl group, wherein at least one skeletal ring atom is a heteroatom selected from nitrogen, oxygen and sulfur. In some embodiments, the radicals are fused with an aryl or heteroaryl. Illustrative examples of heterocycloalkyl groups, also referred to as non-aromatic heterocycles, include:

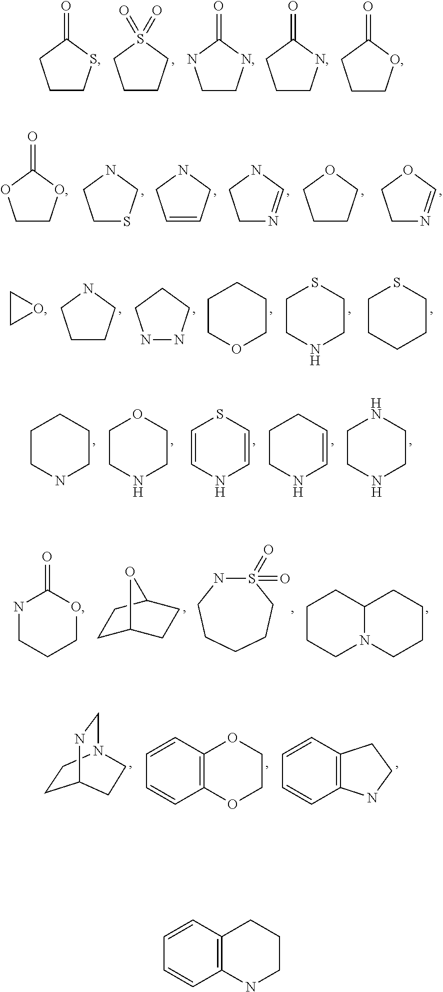

and the like. The term heteroalicyclic also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e skeletal atoms of the heterocycloalkyl ring).

In some embodiments, substituted or unsubstituted heterocycloalkyl groups are selected from among quinolizinyl, dioxinyl, piperidinyl, morpholinyl, thiomorpholinyl, thiazinyl, tetrahydropyridinyl, piperazinyl, oxazinanonyl, dihydropyrrolyl, dihydroimidazolyl, tetrahydrofuranyl, tetrahydropyranyl, dihydrooxazolyl, oxiranyl, pyrrolidinyl, pyrazolidinyl, dihydrothienyl, imidazolidinonyl, pyrrolidinonyl, dihydrofuranonyl, dioxolanonyl, thiazolidinyl, piperidinonyl, indolinyl, indanyl, tetrahydronaphthalenyl, tetrahydroquinolinyl, tetrahydroisoquinolinyl, and tetrahydrothienyl. In other embodiments, substituted or unsubstituted heterocycloalkyl groups are selected from among piperidinyl, morpholinyl, piperazinyl, dihydropyrrolyl, dihydroimidazolyl, tetrahydrofuranyl, dihydrooxazolyl, pyrrolidinyl, pyrazolidinyl, dihydrothienyl, imidazolidinonyl, pyrrolidinonyl, piperidinonyl, indolinyl, indanyl, tetrahydronaphthalenyl, tetrahydroquinolinyl, tetrahydroisoquinolinyl, and tetrahydrothienyl. In yet other embodiments, substituted or unsubstituted heterocycloalkyl groups are selected from among piperidinyl, morpholinyl, piperazinyl, tetrahydrofuranyl, pyrrolidinyl, pyrrolidinonyl, piperidinonyl, indolinyl, indanyl, tetrahydronaphthalenyl, tetrahydroquinolinyl, and tetrahydrothienyl.

“Heterocycloalkylalkyl” refers to an alkyl, as defined herein, substituted with a heterocycloalkyl, as defined herein.

As used herein, “1,3-substituted-1H-indole-6-carboxylic acid hydroxyamide” or “1,3-substituted-1H-indole-6-hydroxamic acid” refers to:

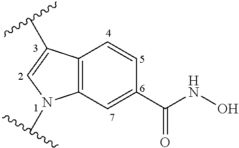 [SMILES: *c1cn(*)c2cc(C(=O)NO)ccc12]

As used herein, “1,3-substituted-1H-indole-5-carboxylic acid hydroxyamide” or “1,3-substituted-1H-indole-5-hydroxamic acid” refers to:

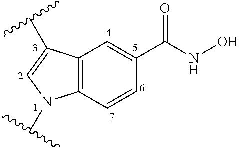 [SMILES: *c1cn(*)c2ccc(C(=O)NO)cc12]

The term “hydroxamate”, “hydroxamic acid”, “N-hydroxycarboxamide” or “carboxylic acid hydroxyamide” refers to:

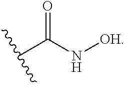 [SMILES: *C(=O)NO]

The term “halo” or, alternatively, “halogen” means fluoro, chloro, bromo and iodo.

The terms “haloalkyl,” “haloalkenyl,” “haloalkynyl” and “haloalkoxy” include alkyl, alkenyl, alkynyl and alkoxy structures that are substituted with one or more halogens. In some embodiments, the halogens are the same or they are different. The terms “fluoroalkyl” and “fluoroalkoxy” include haloalkyl and haloalkoxy groups, respectively, in which the halogen is fluorine. Non-limiting examples of haloalkyls include —CH₂Cl, —CF₃, —CHF₂, —CH₂CF₃, —CF₂CF₃, —CF(CH₃)₃, and the like. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of haloalkoxy groups include —OCF₃, —OCHF₂, —OCH₂F, —OCH₂CF₃, —OCF₂CF₃, —OCF₂CF₂CF₃, —OCF(CH₃)₃, and the like.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus, silicon, or combinations thereof. In some other embodiments, the heteroatom(s) are placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, in other embodiments, up to two heteroatoms are consecutive, such as, by way of example, —CH₂—NH—OCH₃ and —CH₂—O—Si(CH₃)₃. Excluding the number of heteroatoms, in some embodiments, a “heteroalkyl” has from 1 to 6 carbon atoms, a “heteroalkenyl” has from 2 to 6 carbons atoms, and a “heteroalkynyl” has from 2 to 6 carbon atoms. Examples of heteroalkyls include but are not limited to, CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₃, —CH₂—S—CH₂—CH₃, —CH₂—CH₂, —S(O)—CH₃, —CH₂—CH₂—S(O)₂—CH₃, —CH₂—CH═N—OCH₃, —CH₂—NH—OCH₃ and —CH₂—O—Si(CH₃)₃.

The term “bond” or “single bond” refers to a chemical bond between two atoms, or two moieties when the atoms joined by the bond are considered to be part of larger substructure.

A “cyano” group refers to a —CN group.

“Sulfonyl” refers to a —S(═O)₂— moiety.

“Carboxy” refers to a —C(═O)OH group.

As used herein, the substituent “R” appearing by itself and without a number designation refers to a substituent selected from among from alkyl, haloalkyl, heteroalkyl, alkenyl, cycloalkyl, cycloalkylalkyl, aryl, arylalkyl, heteroaryl (bonded through a ring carbon), heteroarylalkyl, heterocycloalkyl, and heterocycloalkylalkyl.

The term “optionally substituted” or “substituted” means that in some embodiments the referenced group is substituted with one or more additional group(s) individually and independently selected from alkyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, cyano, halogen, acyl, acyloxy, isocyanato, thiocyanato, isothiocyanato, nitro, haloalkyl, fluoroalkyl, and amino, including mono- and di-substituted amino groups (e.g. —NH₂, —NHR, —N(R)₂), and the protected derivatives thereof. In some embodiments, an optional substituents L^{s}R^{s}, where each L^{s} is in dependently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₁-C₆alkyl)-, or —(C₂-C₆alkenyl)-; and each R^{s} is independently selected from among H, (C₁-C₆alkyl), (C₃-C₈cycloalkyl), aryl, heteroaryl, heterocycloalkyl, and C₁-C₆heteroalkyl. In other embodiments, the protecting groups that form the protective derivatives of the above substituents include those found in, e.g., Greene and Wuts, above.

In some embodiments, the compounds presented herein possess one or more stereocenters and each center exists in the R or S configuration. The compounds presented herein include all diastereomeric, enantiomeric, and epimeric forms as well as the appropriate mixtures thereof. In other embodiments, stereoisomers are obtained, by methods such as, the separation of stereoisomers by chiral chromatographic columns.

The methods and formulations described herein include the use of N-oxides, crystalline forms (also known as polymorphs), or pharmaceutically acceptable salts of compounds, as well as active metabolites of these compounds having the same type of activity. In some situations, compounds exist as tautomers. All tautomers are included within the scope of the compounds presented herein. In addition, in some embodiments, the compounds described herein exist in unsolvated as well as solvated forms with pharmaceutically acceptable solvents such as water, ethanol, and the like. The solvated forms of the compounds presented herein are also considered to be disclosed herein.

### Examples of Pharmaceutical Compositions and Methods of Administration

In other embodiments, pharmaceutical compositions are formulated in a conventional manner using one or more physiologically acceptable carriers including excipients and auxiliaries which facilitate processing of the active compounds into preparations which are used pharmaceutically. Proper formulation is dependent upon the route of administration chosen. In other embodiments, a summary of pharmaceutical excipients described herein are found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

Provided herein are pharmaceutical compositions that include a compound described herein, and a pharmaceutically acceptable diluent(s), excipient(s), or carrier(s). In addition, in other embodiments, the compounds described herein are administered as pharmaceutical compositions in which compounds described herein are mixed with other active ingredients, as in combination therapy. In some embodiments, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers. In addition, in other embodiments, the pharmaceutical compositions also contain other therapeutically valuable substances.

In certain embodiments, compositions also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

A pharmaceutical composition, as used herein, refers to a mixture of a compound described herein, such as, for example, compounds described herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. In other embodiments, a therapeutically effective amount varies widely depending on the severity of the disease, the age and relative health of the subject, the potency of the compound used and other factors. In further embodiments, the compounds are used singly or in combination with one or more therapeutic agents as components of mixtures.

In some embodiments, the pharmaceutical formulations described herein are administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In other embodiments, pharmaceutical compositions including a compound described herein are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein, such as, for example, a compound described herein, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds exist as tautomers. All tautomers are included within the scope of the compounds presented herein. Additionally, in other embodiments, the compounds described herein exist in unsolvated as well as solvated forms with pharmaceutically acceptable solvents such as water, ethanol, and the like. The solvated forms of the compounds presented herein are also considered to be disclosed herein.

“Antifoaming agents” reduce foaming during processing which can result in coagulation of aqueous dispersions, bubbles in the finished film, or generally impair processing. Exemplary anti-foaming agents include silicon emulsions or sorbitan sesquioleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

“Bioavailability” refers to the percentage of the weight of compounds disclosed herein, such as, compounds described herein, that is delivered into the general circulation of the animal or human being studied. The total exposure (AUC(0-∞)) of a drug when administered intravenously is usually defined as 100% bioavailable (F %). “Oral bioavailability” refers to the extent to which compounds disclosed herein, such as, compounds described herein, are absorbed into the general circulation when the pharmaceutical composition is taken orally as compared to intravenous injection.

“Blood plasma concentration” refers to the concentration of compounds disclosed herein, such as, compounds described herein, in the plasma component of blood of a subject. It is understood that the plasma concentration of compounds described herein may vary significantly between subjects, due to variability with respect to metabolism and/or possible interactions with other therapeutic agents. In accordance with one embodiment disclosed herein, the blood plasma concentration of the compounds described herein may vary from subject to subject. Likewise, values such as maximum plasma concentration (Cmax) or time to reach maximum plasma concentration (Tmax), or total area under the plasma concentration time curve (AUC(0-∞)) may vary from subject to subject. Due to this variability, the amount necessary to constitute “a therapeutically effective amount” of a compound described herein may vary from subject to subject.

“Carrier materials” include any commonly used excipients in pharmaceutics and should be selected on the basis of compatibility with compounds disclosed herein, such as, compounds described herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm3, e.g. Avicel, powdered cellulose), and talc.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cyclamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, □g, or ng of therapeutic agent per ml, dl, or l of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In some embodiments, the compositions described herein are formulated for administration to a subject via any conventional means including, but not limited to, oral, parenteral (e.g., intravenous, subcutaneous, or intramuscular), buccal, intranasal, rectal, topical or transdermal administration routes. By “transdermal” delivery is intended both transdermal (or “percutaneous”) and transmucosal administration, i.e., delivery by passage of a drug through the skin or mucosal tissue and into the bloodstream. Transdermal also refers to the skin as a portal for the administration of drugs or compounds by topical application of the drug or compound thereto. The term “topical application”, as used herein, refers to administration to a surface, such as the skin. This term is used interchangeably with “cutaneous application”. As used herein, the term “subject” is used to mean an animal, in some embodiments a mammal, including a human or non-human. The terms patient and subject are used interchangeably.

Moreover, in some embodiments, the pharmaceutical compositions described herein, which include a compound described herein, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions are used, which are optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, in some embodiments, pharmaceutical formulations of the compounds described herein are administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of a compound described herein, with one or more pharmaceutical excipients to form a bulk blend composition. When referring to these bulk blend compositions as homogeneous, it is meant that the particles of the compound described herein, are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., “The Theory and Practice of Industrial Pharmacy” (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein, and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of a compound described herein. In one embodiment, some or all of the particles of a compound described herein are coated. In another embodiment, some or all of the particles of the compound described herein are microencapsulated. In still another embodiment, the particles of the compound described herein are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound described herein from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of the compound described herein from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule is prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of the compound described herein and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with compounds described herein, which sufficiently isolate the compound described herein from other non-compatible excipients. Materials compatible with compounds described herein are those that delay the release of the compounds described herein in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compounds described herein are formulated by methods such as, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating are also be used.

In one embodiment, the particles of compounds described herein are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds described herein are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound described herein described herein are formulated to include one or more pharmaceutical excipients and flavors. Such a powder is prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When such salts are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In other embodiments, the formulations described herein, which include a compound described herein, are solid dispersions. Methods of producing such solid dispersions include, for example, U.S. Pat. Nos. 4,343,789, 5,340,591, 5,456,923, 5,700,485, 5,723,269, and U.S. Pub. Appl 2004/0013734. In still other embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions include, for example, U.S. Pat. Nos. 4,151,273, 5,281,420, and 6,083,518.

The pharmaceutical solid oral dosage forms including formulations described herein, which include a compound described herein, can be further formulated to provide a controlled release of the compound described herein. Controlled release refers to the release of the compound of Formula (Ia), Formula (IIa), Formula (Ib), or Formula (IIb) from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating for the methods and compositions described herein to achieve delivery to the lower gastrointestinal tract. In some embodiments such polymers are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH >7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;

Poly Vinyl Acetate Phthalate (PVAP). PVAP dissolves in pH >5, and it is much less permeable to water vapor and gastric fluids.

In some embodiments, the coating can, and usually does, contain a plasticizer and possibly other coating excipients such as colorants, talc, and/or magnesium stearate. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) are added to the coatings besides plasticizers to solubilize or disperse the coating material, and to improve coating performance and the coated product.

In other embodiments, the formulations described herein, which include a compound described herein, are delivered using a pulsatile dosage form. A pulsatile dosage form is capable of providing one or more immediate release pulses at predetermined time points after a controlled lag time or at specific sites. Pulsatile dosage forms including the formulations described herein, which include a compound described herein, are administered using a variety of pulsatile formulations such as, but not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284. In one embodiment, the controlled release dosage form is pulsatile release solid oral dosage form including at least two groups of particles, (i.e. multiparticulate) each containing the formulation described herein. The first group of particles provides a substantially immediate dose of the compound described herein upon ingestion by a mammal. The first group of particles can be either uncoated or include a coating and/or sealant. The second group of particles includes coated particles, which includes from about 2% to about 75%, preferably from about 2.5% to about 70%, and more preferably from about 40% to about 70%, by weight of the total dose of the compound described herein in said formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, and Eudragit® NE30D, Eudragit® NE 40D®) either alone or blended with cellulose derivatives, e.g., ethylcellulose, or non-enteric coatings having variable thickness to provide differential release of the formulation that includes a compound described herein.

Many other types of controlled release systems are suitable for use with the formulations described herein. Examples of such delivery systems include, e.g., polymer-based systems, such as polylactic and polyglycolic acid, polyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of the compounds described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of compound described herein, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cyclamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

Intranasal formulations are known in the art and are described in, for example, U.S. Pat. Nos. 4,476,116, 5,116,817 and 6,391,452. Formulations that include a compound described herein, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds of Formula (Ia), Formula (IIa), Formula (Ib), or Formula (IIb) described herein may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Buccal formulations that include compounds described herein may be administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery of the compound described herein, is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the compound described herein, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

In some embodiments, transdermal formulations described herein are administered using a variety of devices which have been described. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

In some embodiments, the transdermal dosage forms described herein incorporate certain pharmaceutically acceptable excipients. In one embodiments, the transdermal formulations described herein include at least three components: (1) a formulation of a compound described herein; (2) a penetration enhancer; and (3) an aqueous adjuvant. In addition, in other embodiments, transdermal formulations include additional components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In other embodiments, formulations suitable for transdermal administration of compounds described herein employ transdermal delivery devices and transdermal delivery patches and are lipophilic emulsions or buffered, aqueous solutions, dissolved and/or dispersed in a polymer or an adhesive. In other embodiments are patches constructed for continuous, pulsatile, or on demand delivery of pharmaceutical agents. Still, in further embodiments, transdermal delivery of the compounds described herein are accomplished by means of iontophoretic patches and the like. Additionally, in other embodiments, transdermal patches provide controlled delivery of the compounds described herein. In another embodiment, the rate of absorption is slowed by using rate-controlling membranes or by trapping the compound within a polymer matrix or gel. Conversely, absorption enhancers are used to increase absorption. In another embodiment, absorption enhancer or carrier includes absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Formulations that include a compound described herein, suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Formulations suitable for topical administration include, but are not limited to, liquid or semi-liquid preparations such as liniments, lotions, oil-in-water or water-in-oil emulsions such as creams, ointments or pastes, and solutions or suspensions. In some embodiments, topically-administrable formulations, for example, comprise from about 1% to about 10% (w/w) active ingredient, although in some embodiments the concentration of the active ingredient is as high as the solubility limit of the active ingredient in the solvent. In other embodiments, formulations for topical administration further comprise one or more of the additional ingredients described herein.

In other embodiments, enhancers of permeation are used. These materials increase the rate of penetration of drugs across the skin. Typical enhancers in the art include ethanol, glycerol monolaurate, PGML (polyethylene glycol monolaurate), dimethylsulfoxide, and the like.

Other enhancers include oleic acid, oleyl alcohol, ethoxydiglycol, laurocapram, alkanecarboxylic acids, dimethylsulfoxide, polar lipids, or N-methyl-2-pyrrolidone.

In other embodiments, an acceptable vehicle for topical delivery of some of the compositions described herein contain liposomes. The composition of the liposomes and their use are known. In other embodiments, the topically active pharmaceutical or cosmetic composition is applied in an amount effective to affect desired changes.

In other embodiments, the topically active pharmaceutical or cosmetic composition are optionally combined with other ingredients such as moisturizers, cosmetic adjuvants, anti-oxidants, chelating agents, bleaching agents, tyrosinase inhibitors and other known depigmentation agents, surfactants, foaming agents, conditioners, humectants, wetting agents, emulsifying agents, fragrances, viscosifiers, buffering agents, preservatives, sunscreens and the like. In another embodiment, a permeation or penetration enhancer is included in the composition and is effective in improving the percutaneous penetration of the active ingredient into and through the stratum corneum with respect to a composition lacking the permeation enhancer. In some embodiments are compositions comprising various permeation enhancers, including oleic acid, oleyl alcohol, ethoxydiglycol, laurocapram, alkanecarboxylic acids, dimethylsulfoxide, polar lipids, or N-methyl-2-pyrrolidone. In other embodiments, the compositions described herein further comprise a hydrotropic agent, which functions to increase disorder in the structure of the stratum corneum, and thus allows increased transport across the stratum corneum. In some other embodiments, are compositions comprising various hydrotropic agents such as isopropyl alcohol, propylene glycol, or sodium xylene sulfonate.

As used herein “amount effective” shall mean an amount sufficient to cover the region of skin surface where a change is desired. In other embodiments, an active compound is present in the amount of from about 0.0001% to about 15% by weight volume of the composition. In other embodiments, it is present in an amount from about 0.0005% to about 5% of the composition; in further embodiments, it is present in an amount of from about 0.001% to about 1% of the composition.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

In some embodiments, the compounds described herein are used in the preparation of medicaments for the inhibition of fatty acid amide hydrolase, or for the treatment of diseases or conditions that would benefit, at least in part, from inhibition of fatty acid amide hydrolase. In addition, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

“Treating” or “treatment” of a disease includes: (1) preventing the disease, i.e. causing the clinical symptoms of the disease not to develop in a mammal that is exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

In other embodiments, the compositions containing the compound(s) described herein are administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. In some embodiments, when used in a patient, effective amounts for this use depend on the severity and course of the disease, disorder or condition, previous therapy, the patient's health status and response to the drugs, and the judgment of the treating physician.

In some embodiments, wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In other embodiments, wherein the patient's status does improve, upon the doctor's discretion the administration of the compounds are given continuously; in other embodiments, the dose of drug being administered is temporarily reduced or temporarily suspended for a certain length of time (i.e., a “drug holiday”). In other embodiments, the length of the drug holiday varies between 2 days and 1 year, including by way of example only, about 2 days, about 3 days, about 4 days, about 5 days, about 6 days, about 7 days, about 10 days, about 12 days, about 15 days, about 20 days, about 28 days, about 35 days, about 50 days, about 70 days, about 100 days, about 120 days, about 150 days, about 180 days, about 200 days, about 250 days, about 280 days, about 300 days, about 320 days, about 350 days, or about 365 days. In other embodiments, the dose reduction during a drug holiday is from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Once improvement of the patient's conditions has occurred, a maintenance dose is administered if necessary. Subsequently, in some embodiments, the dosage or the frequency of administration, or both, is reduced, as a function of the symptoms, to a level at which the improved disease, disorder or condition is retained. In other embodiments, patients require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but nevertheless are routinely determined in a manner known according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1 to about 1500 mg per day. In other embodiments, the desired dose is presented in a single dose or as divided doses administered simultaneously (or over a short period of time) or at appropriate intervals, for example as two, three, four or more sub-doses per day.

In some embodiments, the pharmaceutical composition described herein are in unit dosage forms suitable for single administration of precise dosages. In unit dosage form, the formulation is divided into unit doses containing appropriate quantities of one or more compound. In other embodiments, the unit dosage is in the form of a package containing discrete quantities of the formulation. Non-limiting examples are packaged tablets or capsules, and powders in vials or ampoules.

The daily dosages appropriate for the compounds described herein described herein are from about 0.01 to about 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. The foregoing ranges are merely suggestive, as the number of variables in regard to an individual treatment regime is large, and considerable excursions from these recommended values are not uncommon. In some embodiments, such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and in some embodiments, is expressed as the ratio between LD₅₀ and ED₅₀. Compounds exhibiting high therapeutic indices are also contemplated. In some embodiments, the data obtained from cell culture assays and animal studies are used in formulating a range of dosage for use in human. In some embodiments, the dosage of such compounds lies within a range of circulating concentrations that include the ED₅₀ with minimal toxicity. In other embodiments, the dosage varies within this range depending upon the dosage form employed and the route of administration utilized.

### Combination Treatments

In other embodiments, the compositions described herein are also used in combination with other therapeutic reagents that are selected for their therapeutic value for the condition to be treated. In general, the compositions described herein and, in embodiments where combinational therapy is employed, other agents do not have to be administered in the same pharmaceutical composition, and in some embodiments, because of different physical and chemical characteristics, have to be administered by different routes. In some embodiments, the initial administration is made according to established protocols, and then, based upon the observed effects, the dosage, modes of administration and times of administration is modified by the skilled clinician.

In some embodiments, it is appropriate to administer at least one compound described herein in combination with another therapeutic agent. By way of example only, if one of the side effects experienced by a patient upon receiving one of the compounds herein, such as a compound described herein is nausea, then in other embodiments, it is appropriate to administer an anti-nausea agent in combination with the initial therapeutic agent. Or, by way of example only, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, by way of example only, in some embodiments the benefit experienced by a patient is increased by administering one of the compounds described herein with another therapeutic agent (which also includes a therapeutic regimen) that also has therapeutic benefit. In any case, regardless of the disease, disorder or condition being treated, in some embodiments, the overall benefit experienced by the patient is additive of the two therapeutic agents or the patient experiences a synergistic benefit.

The particular choice of compounds used will depend upon the diagnosis of the attending physicians and their judgment of the condition of the patient and the appropriate treatment protocol. In some embodiments, the compounds are administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

In some embodiments, the therapeutically-effective dosages varies when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, is contemplated herein. Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, in other embodiments, when co-administered with one or more biologically active agents, the compound provided herein is administered either simultaneously with the biologically active agent(s), or sequentially. If administered sequentially, the attending physician will decide on the appropriate sequence of administering protein in combination with the biologically active agent(s).

In some embodiments, the multiple therapeutic agents (one of which is a compound of Formula Ia, Ib, IIa, or IIb described herein) are administered in any order or simultaneously. In some embodiments, wherein the administration is simultaneous, the multiple therapeutic agents are provided in a single, unified form, or in multiple forms (by way of example only, either as a single pill or as two separate pills). In other embodiments, one of the therapeutic agents is given in multiple doses, or both are given as multiple doses. In some other embodiments wherein the administration is not simultaneous, the timing between the multiple doses varies from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, the dosage regimen actually employed varied widely and therefore deviates from the dosage regimens set forth herein.

In some embodiments, the pharmaceutical agents which make up the combination therapy disclosed herein are a combined dosage form or in separate dosage forms intended for substantially simultaneous administration. In further embodiments, the pharmaceutical agents that make up the combination therapy are also administered sequentially, with either therapeutic compound being administered by a regimen calling for two-step administration. In other embodiments, the two-step administration regimen calls for sequential administration of the active agents or spaced-apart administration of the separate active agents. In yet a further embodiment, the time period between the multiple administration steps ranges from, a few minutes to several hours, depending upon the properties of each pharmaceutical agent, such as potency, solubility, bioavailability, plasma half-life and kinetic profile of the pharmaceutical agent. In other embodiments, circadian variation of the target molecule concentration also determines the optimal dose interval.

In addition, in other embodiments, the compounds described herein are used in combination with procedures that provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

In some embodiments, the compounds described herein and combination therapies are administered before, during or after the occurrence of a disease or condition, and the timing of administering the composition containing a compound varies. Thus, for example, in other embodiments, the compounds are used as a prophylactic and are administered continuously to subjects with a propensity to develop conditions or diseases in order to prevent the occurrence of the disease or condition. In further embodiments, the compounds and compositions are administered to a subject during or as soon as possible after the onset of the symptoms. In other embodiments, the administration of the compounds are initiated within the first 48 hours of the onset of the symptoms, in some embodiments, within the first 48 hours of the onset of the symptoms, in further embodiments, within the first 6 hours of the onset of the symptoms, and in other embodiments, within 3 hours of the onset of the symptoms. In another embodiment, the initial administration is via any route practical, such as, for example, an intravenous injection, a bolus injection, infusion over 5 minutes to about 5 hours, a pill, a capsule, transdermal patch, buccal delivery, and the like, or combination thereof. In another embodiment, the compound is administered as soon as is practicable after the onset of a disease or condition is detected or suspected, and for a length of time necessary for the treatment of the disease, such as, for example, from about 1 month to about 3 months. In yet a further embodiment, the length of treatment varies for each subject, and the length is determined using the known criteria. For example, in some embodiments, the compound or a formulation containing the compound is administered for at least 2 weeks, in some embodiments, about 1 month to about 5 years, and in other embodiments, from about 1 month to about 3 years.

### Agents for Treating Autoimmune Diseases, Inflammatory Diseases, or Allergy Diseases

In some embodiments, where the subject is suffering from or at risk of suffering from an autoimmune disease, an inflammatory disease, or an allergy disease, a selective HDAC8 inhibitor compound is administered in any combination with one or more of the following therapeutic agents: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

In one embodiment, selective HDAC8 inhibitor compounds described herein, or compositions and medicaments that include the selective HDAC8 inhibitor compounds described herein, are administered to a patient in combination with an anti-inflammatory agent including, but not limited to, non-steroidal anti-inflammatory drugs (NSAIDs) and corticosteroids (glucocorticoids).

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, fluorobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, CS-502, JTE-522, L-745,337 and NS398).

Combinations with NSAIDs, which are selective COX-2 inhibitors, are contemplated herein. Such compounds include, but are not limited to those disclosed in U.S. Pat. No. 5,474,995; U.S. Pat. No. 5,861,419; U.S. Pat. No. 6,001,843; U.S. Pat. No. 6,020,343, U.S. Pat. No. 5,409,944; U.S. Pat. No. 5,436,265; U.S. Pat. No. 5,536,752; U.S. Pat. No. 5,550,142; U.S. Pat. No. 5,604,260; U.S. Pat. No. 5,698,584; U.S. Pat. No. 5,710,140; WO 94/15932; U.S. Pat. No. 5,344,991; U.S. Pat. No. 5,134,142; U.S. Pat. No. 5,380,738; U.S. Pat. No. 5,393,790; U.S. Pat. No. 5,466,823; U.S. Pat. No. 5,633,272; and U.S. Pat. No. 5,932,598; all of which are hereby incorporated by reference. Other examples of specific inhibitors of COX-2 include those disclosed in U.S. Pat. No. 6,313,138 the disclosure of which is incorporated herein by reference in its entirety.

Compounds that have been described as selective COX-2 inhibitors and are therefore useful in the methods or pharmaceutical compositions described herein include, but are not limited to, celecoxib, rofecoxib, lumiracoxib, etoricoxib, valdecoxib, and parecoxib, or a pharmaceutically acceptable salt thereof.

Corticosteroids, include, but are not limited to: betamethasone (Celestone®), prednisone (Deltasone®), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

Other agents used as anti-inflammatories include those disclosed in U.S. patent publication 2005/0227929, herein incorporated by reference.

Some commercially available anti-inflammatories include, but are not limited to: Arthrotec® (diclofenac and misoprostol), Asacol®, Salofalk® (5-aminosalicyclic acid), Auralgan® (antipyrine and benzocaine), Azulfidine® (sulfasalazine), Daypro® (oxaprozin), Lodine® (etodolac), Ponstan® (mefenamic acid), Solumedrol® (methylprednisolone), Bayer®, Bufferin® (aspirin), Indocin® (indomethacin), Vioxx® (rofecoxib), Celebrex® (celecoxib), Bextra® (valdecoxib), Arcoxia® (etoricoxib), Prexige® (lumiracoxib), Advil®, Motrin® (ibuprofen), Voltaren® (diclofenac), Orudis® (ketoprofen), Mobic® (meloxicam), Relafen® (nabumetone), Aleve®, Naprosyn® (naproxen), Feldene® (piroxicam).

In one embodiment, HDAC8 selective inhibitors are administered in combination with leukotriene receptor antagonists including, but are not limited to, BAY u9773, Cuthbert et al EP 00791576 (published 27 Aug. 1997), DUO-LT (Tsuji et al, Org. Biomol. Chem., 1, 3139-3141, 2003), zafirlukast (Accolate®), montelukast (Singulair®), prankulast (Onon®), and derivatives or analogs thereof.

### EXAMPLES

The following specific examples are to be construed as merely illustrative, and not limitative of the remainder of the disclosure in any way whatsoever.

### Example 1

### Selective HDAC8 Inhibitor Compounds

Candidate selective HDAC8 inhibitor compounds, Compound 23 and Compound 33 were assayed for their ability to inhibit, in vitro, HDAC8, as well as HDACs 1, 2, 3, 6, and 10. For comparison, broad spectrum HDAC inhibitors, CRA-024781 and SAHA, were also assayed in parallel. The results are summarized in Table 4 below. Compound 23 and Compound 33 have HDAC8 IC₅₀ values that are approximately 300 and 15 fold lower, respectively, than the next lowest HDAC target IC₅₀. (as a reference for IC50 determination see Schultz et. al., Biochemistry 43, 11083-11091).

TABLE 4
Comparison of HDAC IC₅₀ values for pan-HDAC vs HDAC8-selective inhibitors

| | HDAC-1 | HDAC-2 | HDAC-3 | HDAC-6 | HDAC-8 | HDAC-10 |
|---|---|---|---|---|---|---|
| | (μM) | (μM) | (μM) | (μM) | (μM) | (μM) |
| 3-((dimethylamino)methyl)-N-(2-(4- | 0.005 | 0.019 | 0.008 | 0.017 | 0.19 | 0.024 |
| (hydroxycarbamoyl)phenoxy)ethyl)benzo- | | | | | | |
| furan-2-carboxamide (broad spectrum) | | | | | | |
| SAHA (broad spectrum) | 0.028 | 0.06 | 0.044 | 0.022 | 0.41 | 0.04 |
| Compound 33 | 2.6 | 23 | 1.5 | 0.36 | 0.024 | 5.3 |
| Compound 23 | 4 | >50 | >50 | 2.9 | 0.010 | 13 |

Based on these data, it was concluded that Compound 23 and Compound 33 are selective inhibitors of HDAC8.

### Example 2

### Compound 23 Inhibits Secretion of IL-1β and Other Pro-Inflammatory Cytokines in LPS-Induced Human PBMCs

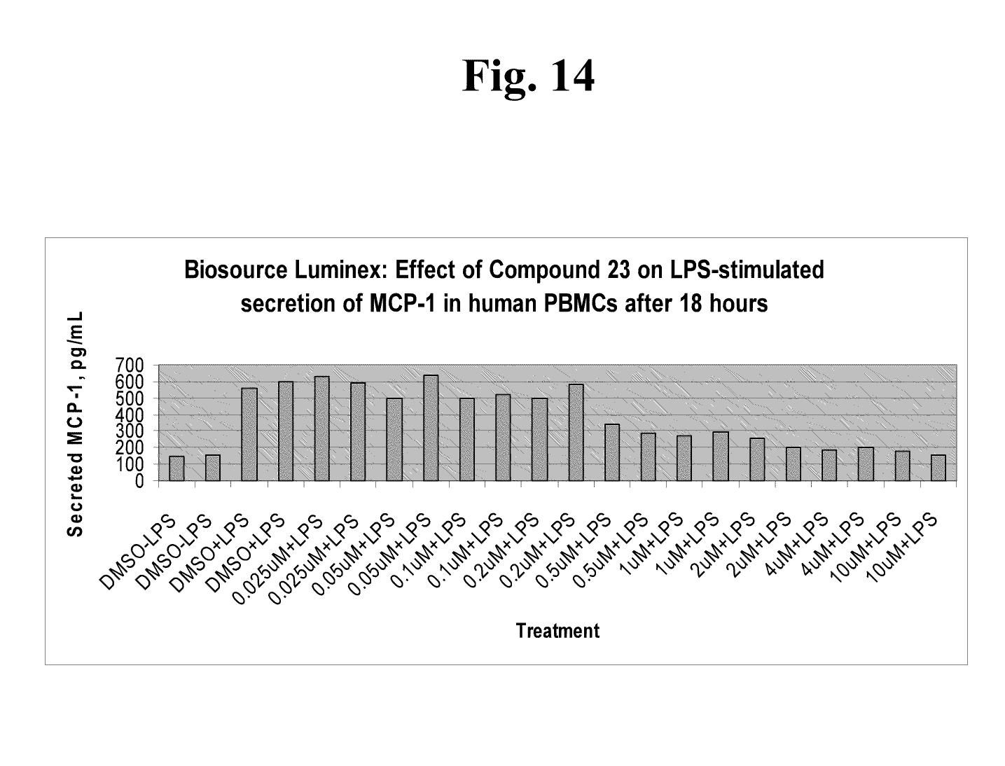

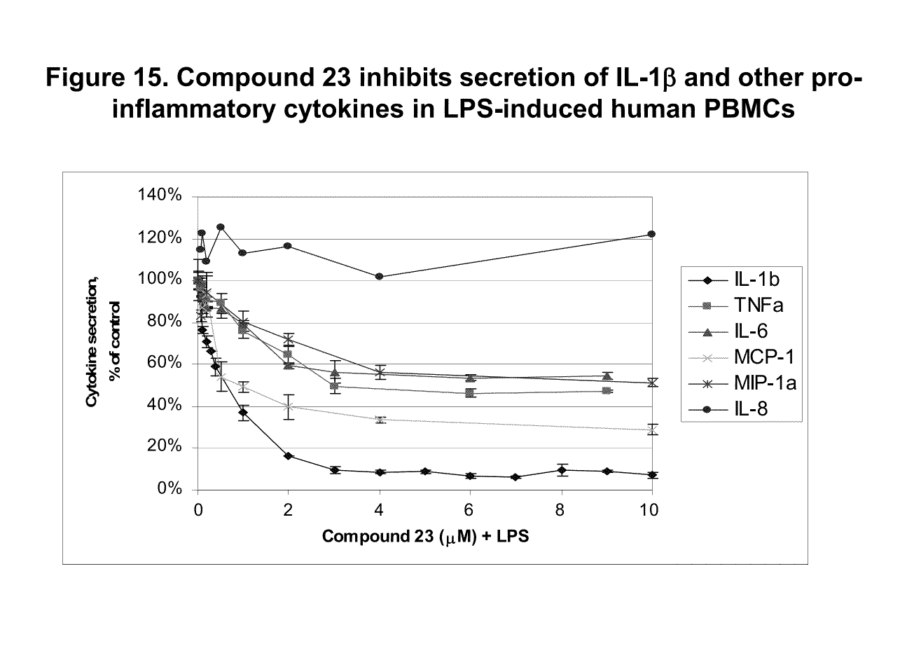

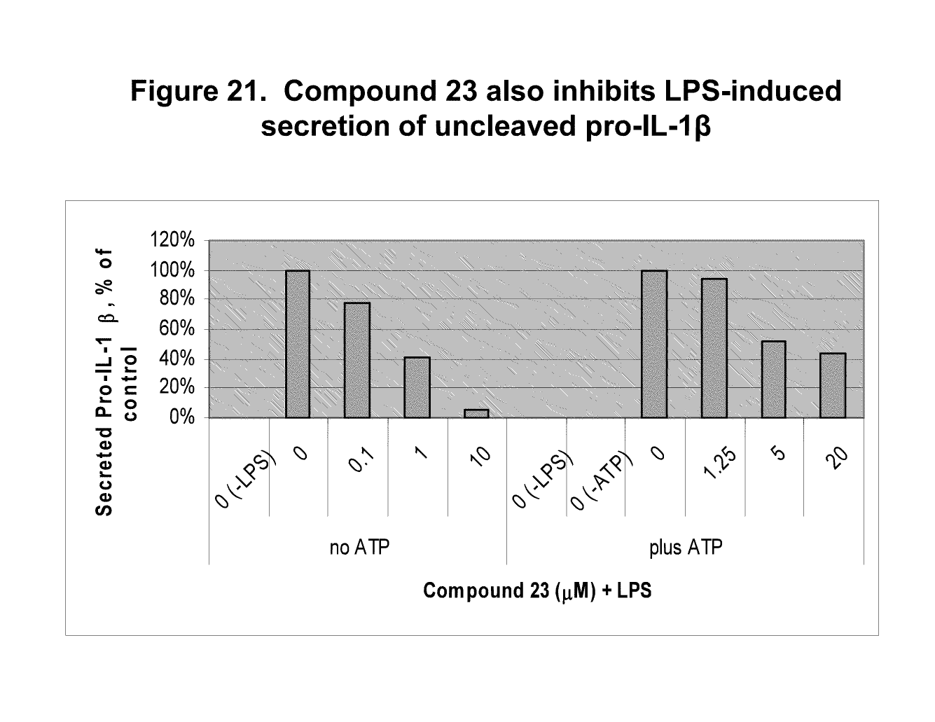

Human PBMCs were pre-treated for 1 hour with various concentrations of Compound 23 before stimulation with 10 ng/mL LPS for an additional 15 hours (FIG. 14). Culture supernatants were analyzed for cytokine levels by ELISA (FIG. 14) or Luminex assay. Compound 23 was found to inhibit secretion of IL-1β, TNFα, IL-6, MCP-1, MIP-1a, but not IL-8. Human PBMCs pre-treated for 1 hour with Compound 23 were also stimulated with 10 ng/mL LPS for an additional 15 hours without ATP or 3 hours followed by 1 mM ATP for 15 minutes, and culture supernatants analyzed for pro-IL-1β protein by ELISA (FIG. 21). Results show that Compound 23 also inhibits LPS-induced secretion of uncleaved pro-IL-1β protein.

### Example 3

### Compound 23 Inhibits Secretion of Multiple Cytokines from Human PBMCs

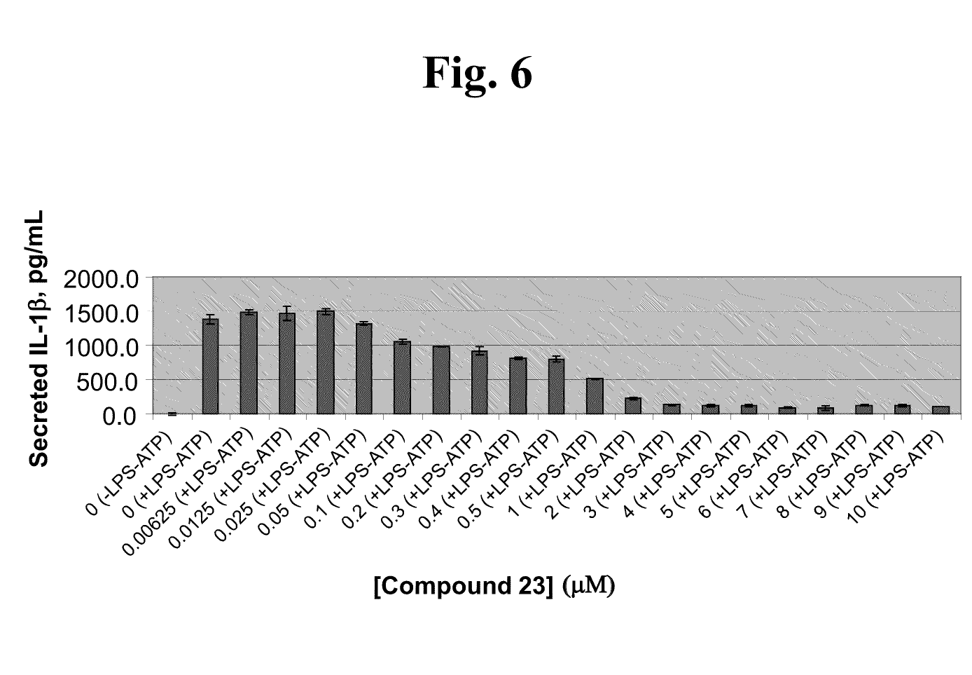

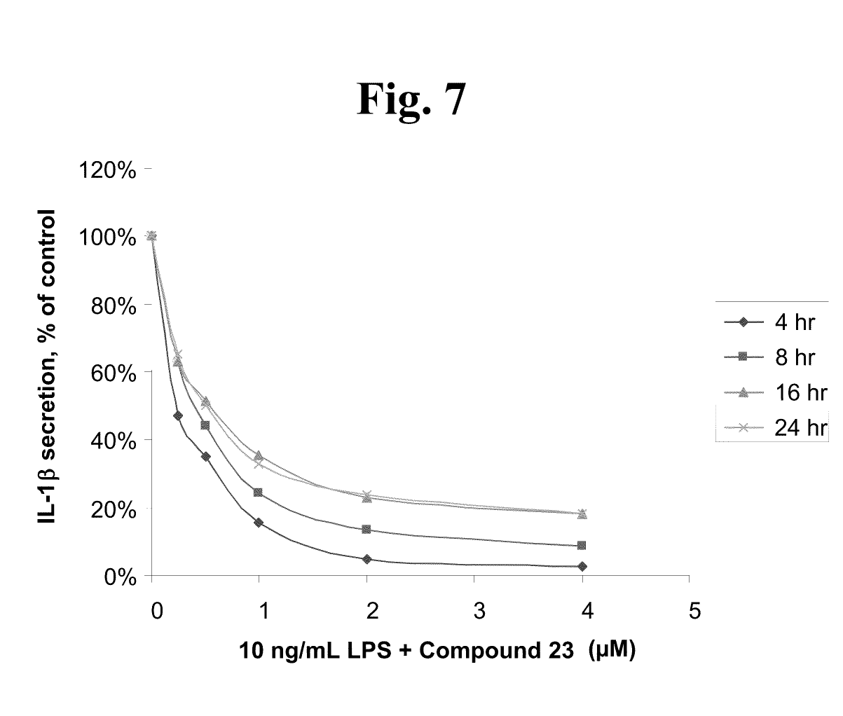

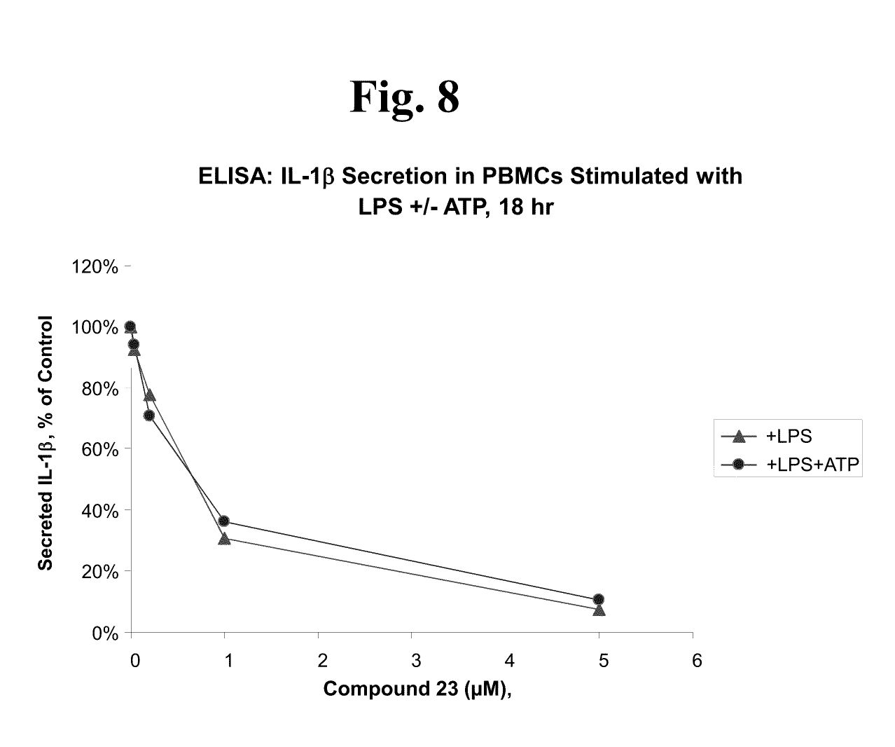

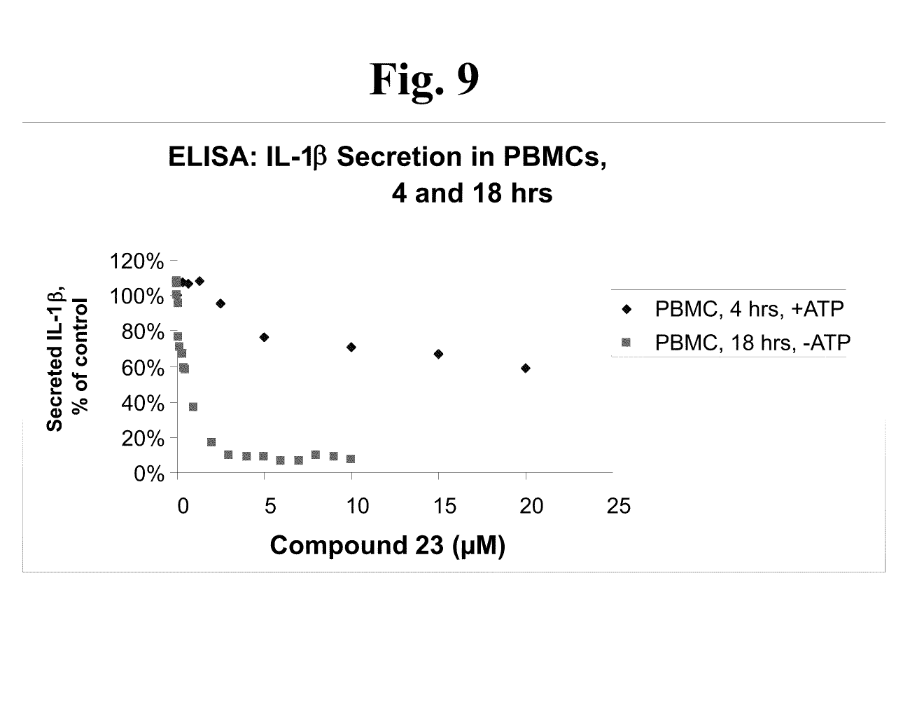

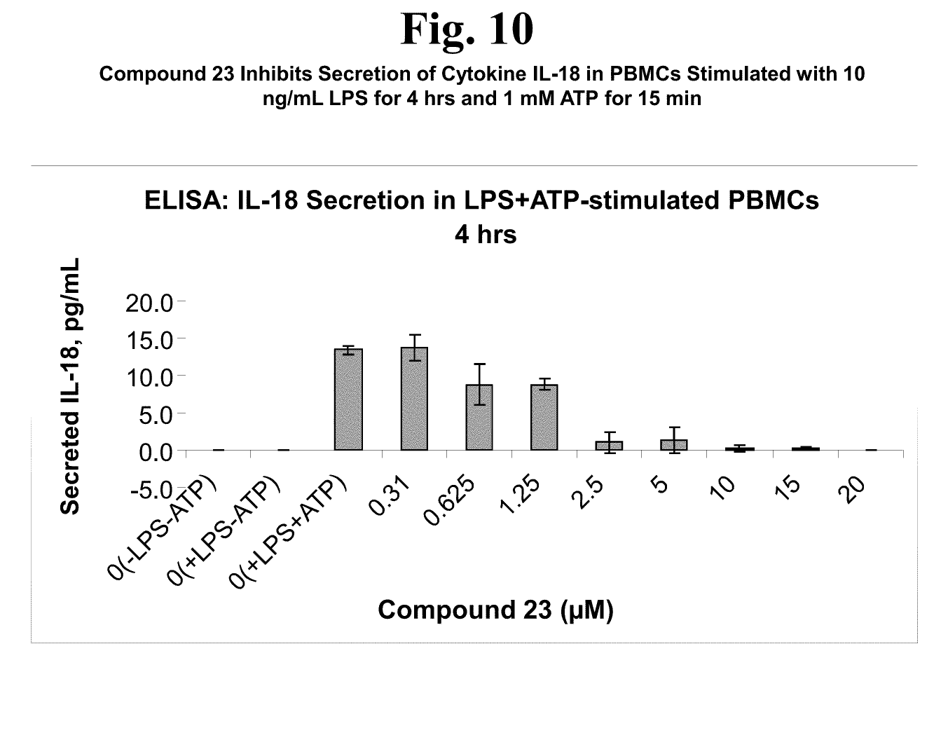

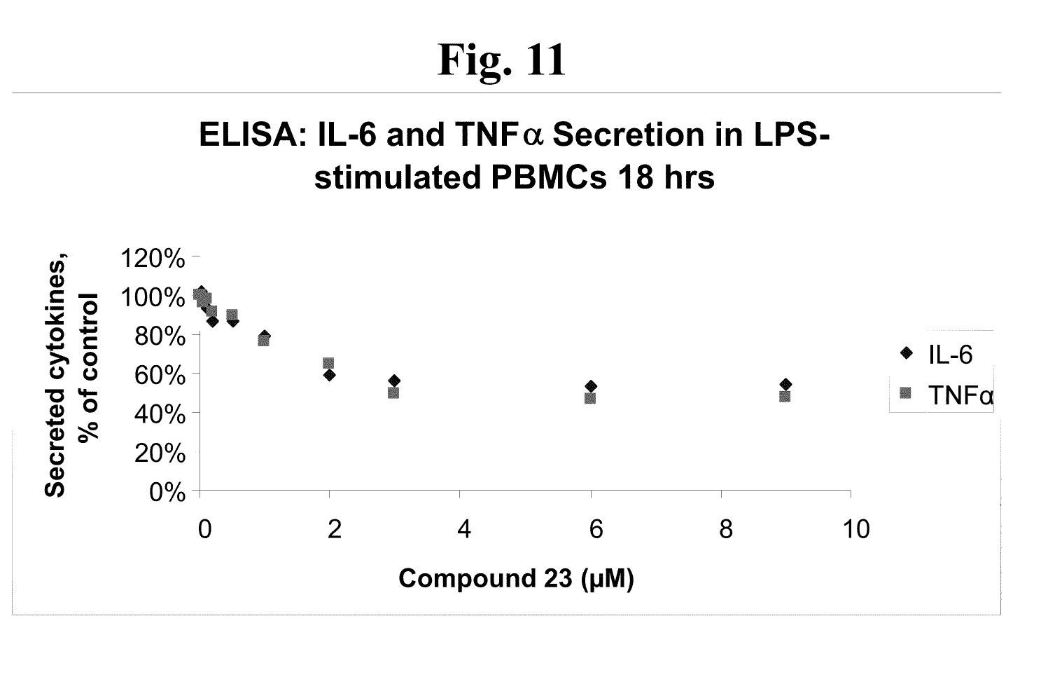

We examined the ability of the HDAC8-selective inhibitor, compound 23, to decrease secretion of cytokines, which are known to play a role in inflammation. To this end, we cultured human PBMCs for varying lengths of time in the presence of the cytokine secretagogues, LPS or LPS plus ATP, along with varying concentrations of compound 23. In one set of experiments, secreted levels of IL-1β were determined by ELISA. As shown in FIGS. 6-9, compound 23 resulted in a robust dose-dependent inhibition of LPS and/or ATP-stimulated secretion of IL-1β in the cultured PBMCs. Similarly, in PBMCs, compound 23 exerted a potent inhibition of LPS plus ATP-stimulated IL-18 secretion (FIG. 10) and LPS-stimulated secretion of IL-6 and TNF-α (FIG. 11). Based on these results we concluded that compound 23 is an effective inhibitor of inflammatory cytokine secretion.

### Example 4

### Compound 23 Inhibits IL-1β Secretion in LPS-Induced Monocytes

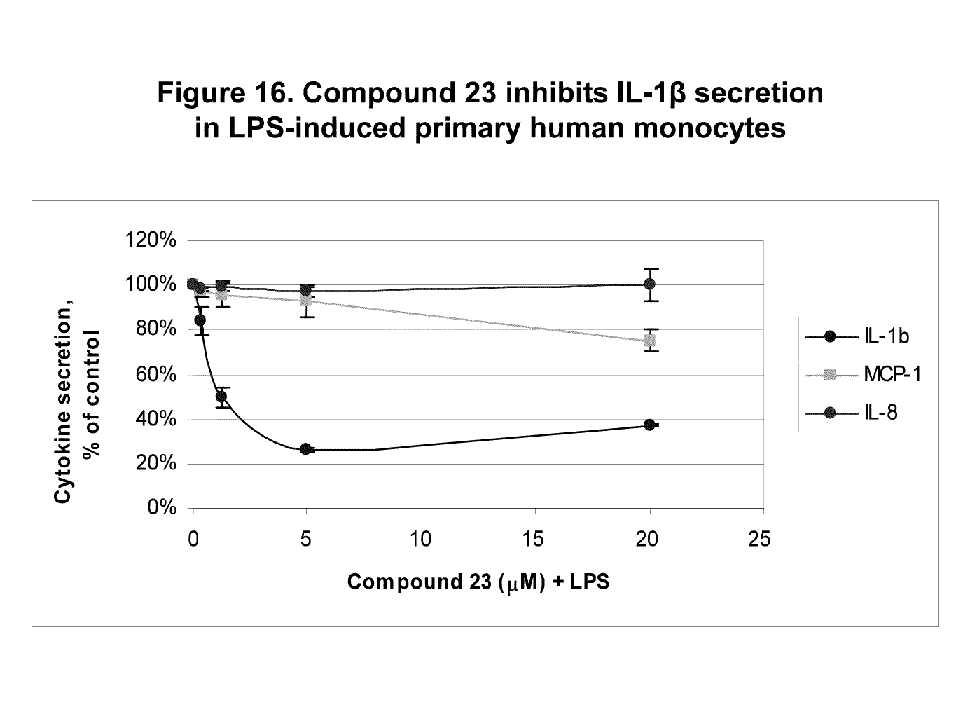

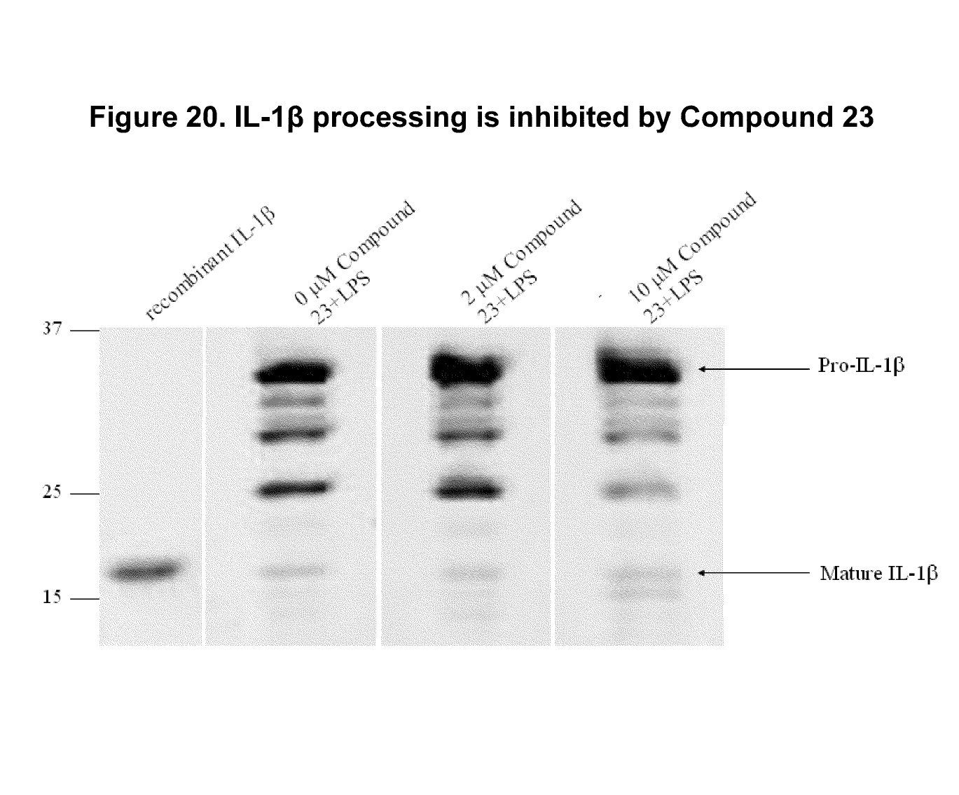

Primary human monocytes isolated by negative selection were pre-treated for 1 hour with the indicated concentrations of Compound 23 before stimulation with 10 ng/ml LPS for an additional 4 hours. Culture supernatants were analyzed for cytokine levels by ELISA (FIG. 16). Results demonstrated that Compound 23 inhibited IL-1β secretion in LPS-induced primary human monocytes. Cell lysates were also analyzed for levels of IL-1β species by western blotting (FIG. 20). Densitometry indicates that pro-IL-1β levels in 2 μM and 10 μM lanes are 153% and 142%, respectively, of control levels. Compound 23 was also found to minimally inhibit IL-1β transcription.

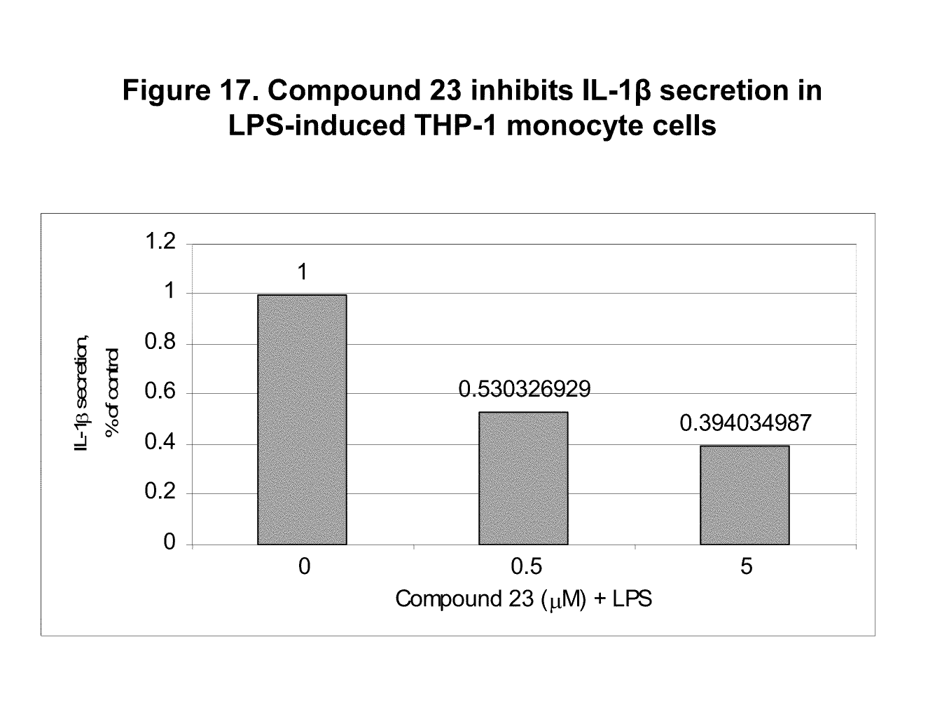

THP-1 monocytic cells were pre-treated for 1 hour with various concentrations of Compound 23 before stimulation with 100 ng/mL LPS for an additional 23 hours. Culture supernatants were analyzed for IL-1β by ELISA (FIG. 17). Results demonstrate that Compound 23 inhibited IL-1β secretion in LPS-induced THP-1 monocyte cells.

### Example 5

### Compound 23 is a More Potent Inhibitor of LPS-Induced IL-1β Secretion than of LPS+ATP Induced IL-1β Secretion

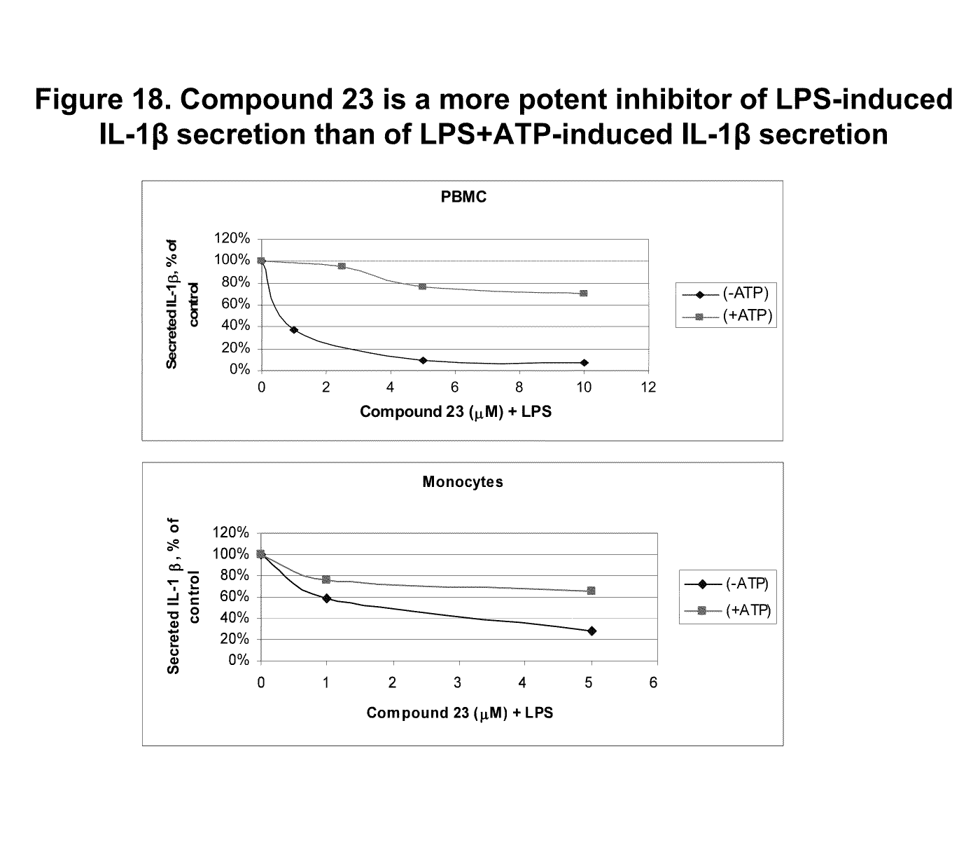

Human PBMCs and primary monocytes were pre-treated for 1 hour with various concentrations of Compound 23 before stimulation with 10 ng/mL LPS for 16 hours (PBMC-ATP) or 4 hours (PBMC+ATP and monocytes). Cells were treated with 1 mM ATP for 10 minutes. Culture supernatants were analyzed for IL-1β by ELISA (FIG. 18). Results indicate that Compound 23 was a more potent inhibitor of LPS-induced IL-1β secretion than of LPS+ATP induced IL-1β secretion.

### Example 6

### Compound 23 does not Directly Inhibit Caspace-1 or TACE Proteases

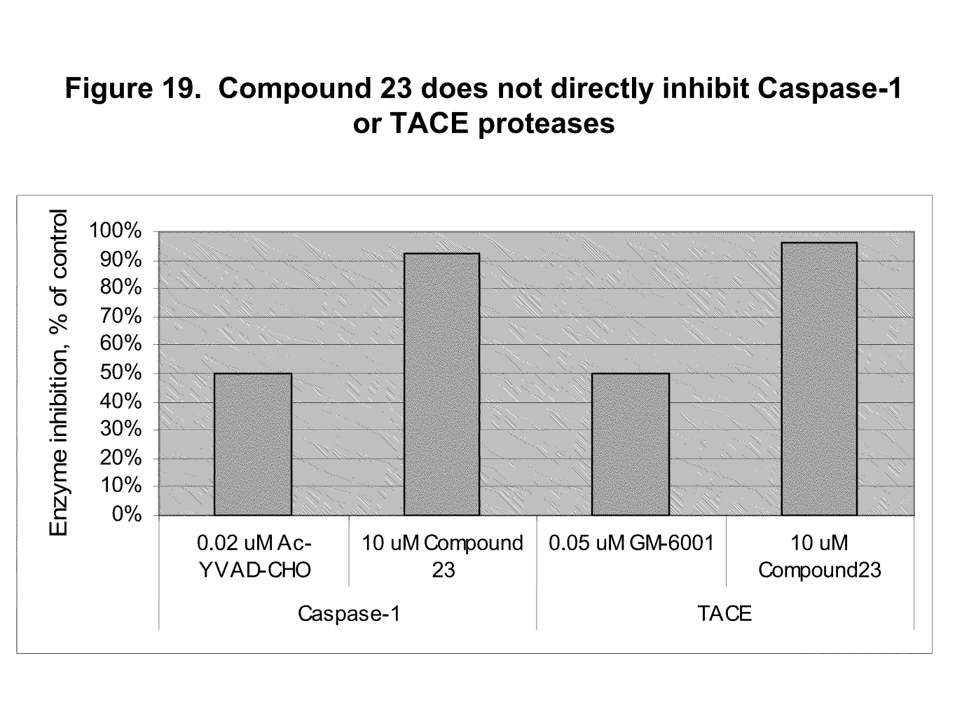

Purified proteases were incubated with substrates and 0.02 μM Ac-YVAD-CHO (a reversible inhibitor of Caspase-1), 10 μM Compound 23, or 0.06 μM GM-6001 (a hydroxamate inhibitor of matrix metalloproteinases including TNFα converting enzyme (TACE)). Product was quantified by fluorescence (n=2) (FIG. 19). Ac-YVAD-CHO and GM-6001 served as positive controls and were used at empirically determined IC₅₀ concentrations.

### Example 7

### Compound 23 Inhibits Inflammation in an In Vivo Model

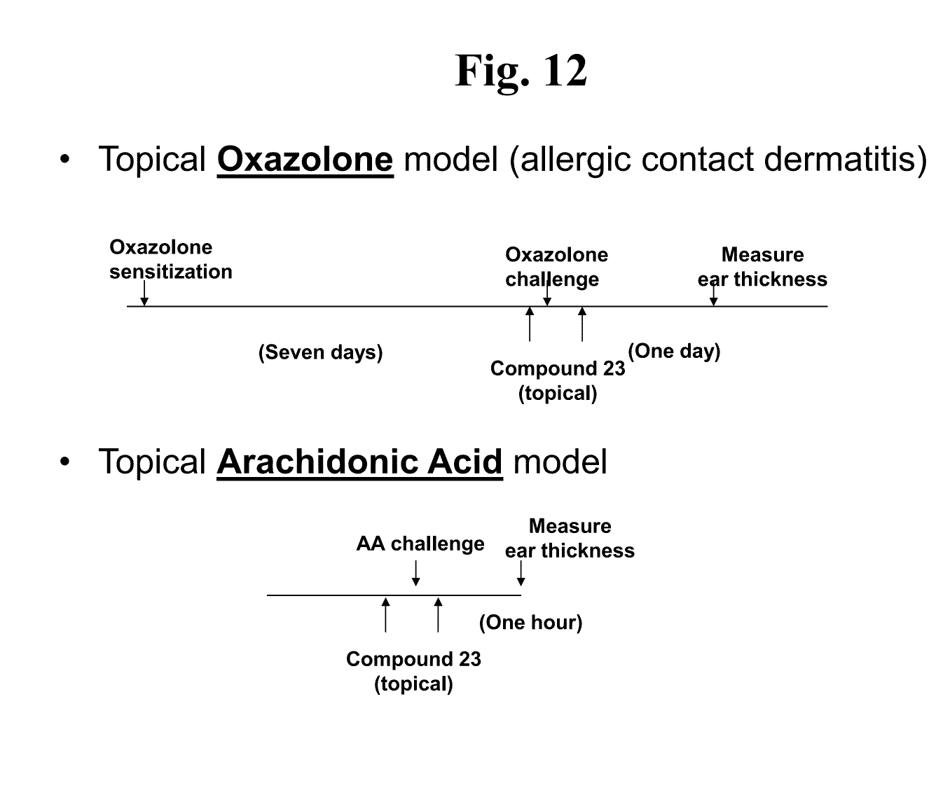

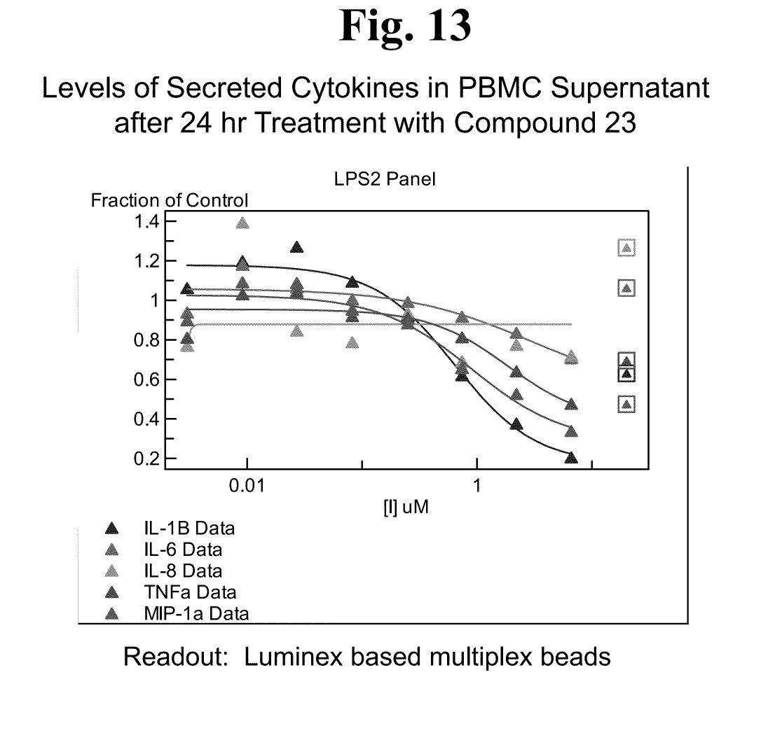

Based on the ability of Compound 23 to inhibit cytokine secretion in vitro, we sought to determine whether this compound exhibits anti-inflammatory properties in vivo. To this end, we utilized two mouse models of allergic contact dermatitis, which are schematically illustrated in FIG. 12. Animals were sensitized on shaved abdomens with 0.1 mL of 1.5% oxazolone in acetone 7 days prior to the study. In the first model, the pro-inflammatory agent, oxazalone, was topically applied to one ear in BALB/c mice, which had been treated before and after oxazolone treatment with a topical formulation containing either a vehicle control, Compound 23, or indomethacin (a known anti-inflammatory agent). Afterwards, the difference in the thickness of each of the two ears was compared to measure the extent of swelling (ie. inflammation). Swelling in the control treated mice was compared to swelling in the compound 23- or indomethacin-treated mice to measure any treatment-associated reduction in inflammation. As shown in Table 5, Compound 23 at 3 mg/ear×2 demonstrated significant anti-inflammatory activity in the oxazolone-induced topical inflammatory model in BALB/c mice.

TABLE 5
Anti-inflammatory activity of compound 23 in the oxazolone-induced
topical inflammatory model in BALB/c mice.

| | | Ear Thickness (0.01 mm) | |
|---|---|---|---|
| Treatment | Dose | Net | % Inhibition* |
| Vehicle | 40 μ1/ear × 2 | 16.2 ± 0.8 | — |
| (Ethanol:Acetone/1:1) | | | |
| Compound 23 | 3 mg/ear × 2 | 7.3 ± 0.3 | (55) |
| Compound 23 | 1 mg/ear × 2 | 12.5 ± 1.8 | 23 |
| Indometacin | 0.3 mg/ear × 2 | 9.7 ± 1.0 | (40) |

*A 30% or more (≧30%) inhibition relative to the vehicle-treated control is considered significant anti-inflammatory activity.

As a further test of the ability of compound 23 to reduce topical inflammation, we utilized an arachidonic acid inflammation model (see FIG. 12). As shown in Table 6, reduced arachidonic acid-induced inflammation was observed for at least one dose (1 mg) of compound 23.

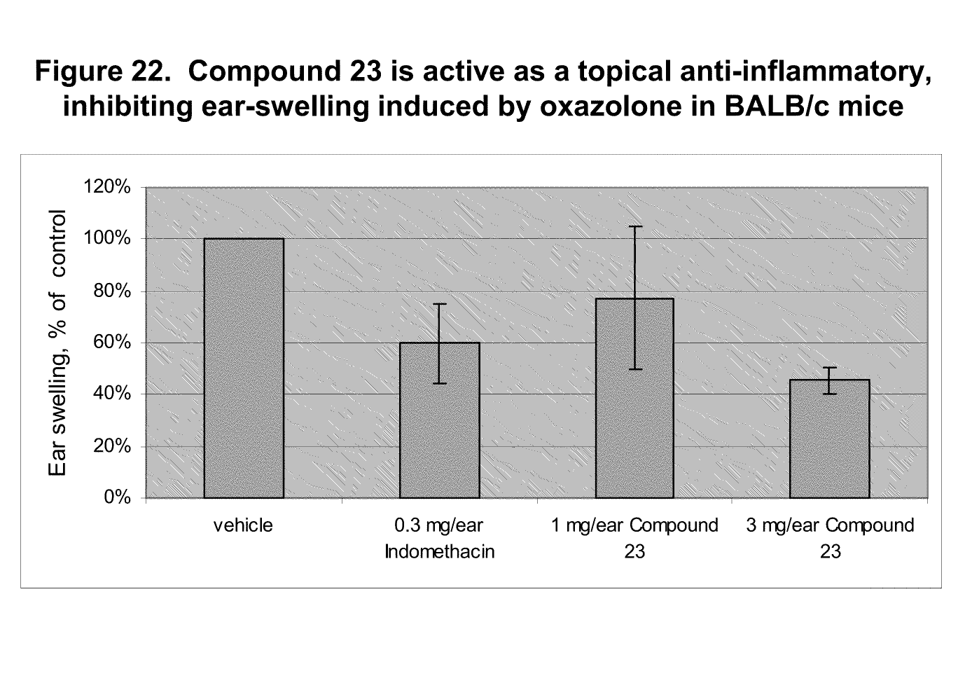

As a further test, vehicle or the indicated compounds were administered topically in 40 μl doses 30 minutes before and 15 minutes after a second challenge (25 μl/ear of 1% oxazolone in acetone) (n=6). Ear thickness was measured 24 hours after the second challenge with a Dyer model micrometer gauge. In this model of inflammation, the standard positive control is indomethacin and ≧30% inhibition of swelling compared to vehicle-treated control is considered significant anti-inflammatory activity. Compound 23 was found active as a topical anti-inflammatory, inhibiting ear-swelling (FIG. 22).

TABLE 6
Anti-inflammatory activity of compound 23 in the arachidonic
acid-induced topical inflammatory model in BALB/c mice.

| | | | | Ear thickness (×0.01 mm) | | | |
|---|---|---|---|---|---|---|---|
| Treatment | Route | Dose | N | R. Ear | L. Ear | Net | % Inhibition |
| Vehicle | TOP | 40μ 10ear × 2 | 1 | 34 | 19 | 15 | — |
| (Ethanol:Acetone/1:1) | | | 2 | 36 | 20 | 16 | |
| | | | 3 | 33 | 20 | 13 | |
| | | | 4 | 32 | 20 | 12 | |
| | | | 5 | 34 | 20 | 14 | |
| | | | 6 | 35 | 19 | 16 | |
| | | | X | 34.0 | 19.7 | 14.3 | |
| | | | SEM | 0.6 | 0.2 | 0.7 | |
| Indomethacin | TOP | 3 mg/ear × 2 | 1 | 26 | 19 | 7 | (62) |
| | | | 2 | 25 | 21 | 4 | |
| | | | 3 | 26 | 20 | 6 | |
| | | | 4 | 26 | 20 | 6 | |
| | | | 5 | 25 | 20 | 5 | |
| | | | 6 | 26 | 21 | 5 | |
| | | | X | 25.7 | 20.2 | 5.5 | |
| | | | SEM | 0.2 | 0.3 | 0.4 | |
| Compound 23 | TOP | 3 mg/ear × 2 | 1 | 27 | 21 | 6 | 28 |
| | | | 2 | 32 | 21 | 11 | |
| | | | 3 | 35 | 19 | 16 | |
| | | | 4 | 27 | 21 | 6 | |
| | | | 5 | 36 | 20 | 16 | |
| | | | 6 | 28 | 21 | 7 | |
| | | | X | 30.8 | 20.5 | 10.3 | |
| | | | SEM | 1.7 | 0.3 | 1.9 | |
| Compound 23 | TOP | 1 mg/ear × 2 | 1 | 29 | 20 | 9 | (32) |
| | | | 2 | 32 | 21 | 11 | |
| | | | 3 | 28 | 19 | 9 | |
| | | | 4 | 28 | 20 | 8 | |
| | | | 5 | 32 | 20 | 12 | |
| | | | 6 | 29 | 20 | 9 | |
| | | | X | 29.7 | 20.0 | 9.7 | |
| | | | SEM | 0.8 | 0.3 | 0.6 | |

Based on these data we concluded that the HDAC8-selective inhibitor compound, compound 23, was effective for inhibiting inflammation in mouse models of allergic contact dermatitis.

Throughout the specification, claims and accompanying figures, a number of embodiments have been described. Nevertheless, it will be understood that various modifications are made without departing from the spirit and scope of the embodiments described herein. Accordingly, other embodiments are within the scope of the following claims.

What is claimed is:

1. A method for treating an inflammatory condition in a subject in need thereof, comprising administering to the subject a composition containing a therapeutically effective amount of a selective inhibitor of histone deacetylase 8 activity.

2. The method of claim 1, wherein (a) the secretion of IL-1β in a sample taken from the subject is inhibited by at least 40%, and/or (b) the swelling on the skin of the subject decreases by at least 30% after administering the therapeutically effective amount of the selective inhibitor of histone deacetylase 8 activity.

3. The method of claim 1, wherein the inflammatory condition is a skin inflammatory condition, autoimmune condition, or heteroimmune condition.

4. The method of claim 1, wherein the inflammatory condition is rheumatoid arthritis or psoriasis.

5. The method of claim 1, wherein the subject is refractory or intolerant to at least one other treatment for an inflammatory condition.

6. The method of claim 1, wherein the composition is administered in combination with an additional anti-inflammatory agent.

7. The method of claim 6, wherein the additional anti-inflammatory agent is an immunosuppressant, glucocorticoid, non-steroidal anti-inflammatory drug, Cox-2 specific inhibitor, leflunomide, gold thioglucose, gold thiomalate, aurofin, sulfasalazine, hydroxychloroquinine, minocycline, TNF-α binding proteins, abatacept, anakinra, interferon-β, interferon-γ, interleukin-2, allergy vaccines, antihistamines, antileukotrienes, beta-agonists, theophylline, anticholinergic, or any combination thereof.

8. The method of claim 1, wherein the composition is administered systemically, locally, or topically.

9. The method of claim 8, wherein the composition is administered topically.

10. The method of claim 1, wherein the selective inhibitor is a 1,3-disubstituted-1H-indole-6-carboxylic acid hydroxyamide compound, wherein the substituent at the 1-position is —X²—R² and the substituent at the 3-position is R³, wherein:
X² is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene; —C(═O)—, and —C(═O)—C₁-C₆alkylene;
R² is a substituted or unsubstituted group selected from among aryl, heteroaryl, cycloalkyl, and heterocycloalkyl;
where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is hydrogen, halogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁶—R⁶;
X⁶ is a C₁-C₆alkylene, C₁-C₆fluoroalkylene, C₂-C₆alkenylene, C₂-C₆heteroalkylene;
R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁷—R⁷
X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁷ together with the N atom to which they are attached form a 5-, 6-, or 7-membered heterocycloalkyl;
or an active metabolite, pharmaceutically acceptable solvate, pharmaceutically acceptable salt, pharmaceutically acceptable N-oxide, or pharmaceutically acceptable prodrug thereof.

11. The method of claim 1, wherein the selective inhibitor is a compound selected from among:
1-(3,4-dichloro-phenylmethyl)-1H-indole-6-carboxylic acid hydroxyamide (Compound 1); 1-(2-methyl-phenylmethyl)-1H-indole-6-carboxylic acid hydroxyamide (Compound 2); 1-(3,4,5-trimethoxy-phenylmethyl)-1H-indole-6-carboxylic acid hydroxyamide (Compound 3); 1-(3-fluoro-phenylmethyl)-1H-indole-6-carboxylic acid hydroxyamide (Compound 4); 1-(3-methyl-phenylmethyl)-1H-indole-6-carboxylic acid hydroxyamide (Compound 5); 1-(benzyl)-1H-indole-6-carboxylic acid hydroxyamide (Compound 6); 1-(3,5-dimethoxy-phenylmethyl)-1H-indole-6-carboxylic acid hydroxyamide (Compound 7); 1-(1-methyl-1-phenylmethyl)-1H-indole-6-carboxylic acid hydroxyamide (Compound 8); 1-(4-fluoro-phenylmethyl)-1H-indole-6-carboxylic acid hydroxyamide (Compound 9); 1-(2-fluoro-phenylmethyl)-1H-indole-6-carboxylic acid hydroxyamide (Compound 10); 1-(2-chloro-phenylmethyl)-1H-indole-6-carboxylic acid hydroxyamide (Compound 11); 1-(3-methoxy-phenylmethyl)-1H-indole-6-carboxylic acid hydroxyamide (Compound 12); 1-(naphth-2-ylmethyl)-1H-indole-6-carboxylic acid hydroxyamide (Compound 13); 1-(3-phenylpropyl)-1H-indole-6-carboxylic acid hydroxyamide (Compound 14); 1-(cyclohexylmethyl)-1H-indole-6-carboxylic acid hydroxyamide (Compound 15); 1-[1-(phenyl)-propen-3-yl]-1H-indole-6-carboxylic acid hydroxyamide (Compound 16); 1-[4-(trifluoromethoxy)-phenylmethyl]-1H-indole-6-carboxylic acid hydroxyamide (Compound 17); 1-(4-chloro-phenylmethyl)-1H-indole-6-carboxylic acid hydroxyamide (Compound 18); 1-(benzo[2,1,3]oxadiazol-5-ylmethyl)-1H-indole-6-carboxylic acid hydroxyamide (Compound 19; 1-(4-methyl-phenylmethyl)-1H-indole-6-carboxylic acid hydroxyamide (Compound 20); 1-(3-fluoro-4-methoxy-phenylmethyl)-1H-indole-6-carboxylic acid hydroxyamide (Compound 21); 1-[4-(difluoromethoxy)-phenylmethyl]-1H-indole-6-carboxylic acid hydroxyamide (Compound 22); 1-(4-methoxy-phenylmethyl)-1H-indole-6-carboxylic acid hydroxyamide (Compound 23); 1-(phenethyl)-1H-indole-6-carboxylic acid hydroxyamide (Compound 24); 1-(3-chloro-phenylmethyl)-1H-indole-6-carboxylic acid hydroxyamide (Compound 25); 1-[N-(t-butoxycarbonyl)piperidin-4-ylmethyl]-1H-indole-6-carboxylic acid hydroxyamide (Compound 26); 1-(piperidin-4-ylmethyl)-1H-indole-6-carboxylic acid hydroxyamide (Compound 27); 1-(N-methylsulfonyl-3-aminobenzyl)-1H-indole-6-carboxylic acid hydroxyamide (Compound 28); 3-(Dimethylaminomethyl)-1-(4-methoxybenzyl)-1H-indole-6-carboxylic acid hydroxyamide (Compound 29); 3-(N-Morpholinomethyl)-1-(4-methoxybenzyl)-1H-indole-6-carboxylic acid hydroxyamide (Compound 30); 3-(N-Pyrrolidinomethyl)-1-(4-methoxybenzyl)-1H-indole-6-carboxylic acid hydroxyamide (Compound 31); 3-(N-Benzylaminomethyl)-1-(4-methoxybenzyl)-1H-indole-6-carboxylic acid hydroxyamide (Compound 32); and 3-(Ethyl)-1-(4-methoxybenzyl)-1H-indole-6-carboxylic acid hydroxyamide (Compound 33).

12. The method of claim 1, wherein the selective inhibitor is a 1,3-disubstituted-1H-indole-5-carboxylic acid hydroxyamide compound, wherein the substituent at the 1-position is R⁴ and the substituent at the 3-position is —X⁵—R⁵, wherein:
R⁴ is hydrogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁸—R⁸;
X⁸ is a C₂-C₆alkylene, C₂-C₆fluoroalkylene, C₂-C₆alkenylene, or C₂-C₆heteroalkylene;
R⁸ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁹—R⁹;
X⁹ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁹ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl, R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁹ together with the N atom to which they are attached form a 5-, 6-, or 7-membered heterocycloalkyl;
X⁵ is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene, —C(═O)—, and —C(═O)—C₁-C₆alkylene;
R⁵ is a substituted or unsubstituted group selected from among aryl, heteroaryl, C₃-C₈cycloalkyl, and heterocycloalkyl;
where if R⁵ is substituted, then each substituent on R⁵ is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl; or an active metabolite, pharmaceutically acceptable solvate, pharmaceutically acceptable salt, pharmaceutically acceptable N-oxide, or pharmaceutically acceptable prodrug thereof.

13. The method of claim 1, wherein the selective inhibitor is selected from among:
1-methyl-3-(4-nitro-phenylmethyl)-1H-indole-5-carboxylic acid hydroxyamide (Compound 34);
1-ethyl-3-(phenylmethyl)-1H-indole-5-carboxylic acid hydroxyamide (Compound 35);
1-methyl-3-[4-(phenylcarbonylamino)-phenylmethyl]-1H-indole-5-carboxylic acid hydroxyamide (Compound 36);
1-isopropyl-3-(phenylmethyl)-1H-indole-5-carboxylic acid hydroxyamide (Compound 37);
1-methyl-3-(4-amino-phenylmethyl)-1H-indole-5-carboxylic acid hydroxyamide (Compound 38);
1-methyl-3-(4-fluoro-phenylmethyl)-1H-indole-5-carboxylic acid hydroxyamide (Compound 39);
1-phenyl-3-(phenylmethyl)-1H-indole-5-carboxylic acid hydroxyamide (Compound 40); and
1-methyl-3-[4-(t-butoxycarbonyl)piperazin-1-ylmethyl]-1H-indole-5-carboxylic acid hydroxyamide (Compound 41).

14. A method for decreasing secretion of a pro-inflammatory cytokine in a subject in need thereof, comprising administering to the subject a pharmaceutical composition comprising therapeutically effective amount of at least one selective inhibitor of histone deacetylase 8 activity.

15. The method of claim 14, wherein the pro-inflammatory cytokine is IL-1β.

16. The method of claim 14, wherein the pro-inflammatory cytokine is TNF-α.

17. The method of claim 14, wherein the pro-inflammatory cytokine is IL-6.

18. The method of claim 14, wherein the pro-inflammatory cytokine is MCP-1.

19. The method of claim 14, wherein the pro-inflammatory cytokine is MIP-1α.

20. The method of claim 14, wherein the at least one selective inhibitor of histone deacetylase 8 activity is a 1,3-disubstituted-1H-indole-6-carboxylic acid hydroxyamide compound, wherein the substituent at the 1-position is —X²—R² and the substituent at the 3-position is R³, wherein:
X² is a bond, or a substituted or unsubstituted group selected from among C₁-C₆alkylene, C₂-C₆alkenylene, C₂-C₆ alkynylene, C₁-C₆fluoroalkylene, C₂-C₆fluoroalkenylene, C₁-C₆haloalkylene, C₂-C₆haloalkenylene, C₁-C₆heteroalkylene; —C(═O)—, and —C(═O)—C₁-C₆alkylene;
R² is a substituted or unsubstituted group selected from among aryl, heteroaryl, cycloalkyl, and heterocycloalkyl;
where if R² is substituted, then each substituent on R² is selected from among hydrogen, halogen, —CN, —NO₂, —S(═O)₂NH₂, —CO₂H, —CO₂R¹⁰, —C(═O)R¹¹, —S—R¹¹, —S(═O)—R¹¹, —S(═O)₂—R¹¹, —NR¹⁰C(═O)—R¹¹, —C(═O)N(R¹⁰)₂, —S(═O)₂N(R¹⁰)₂, —NR¹⁰S(═O)₂—R¹¹, —OC(═O)N(R¹⁰)₂, —NR¹⁰C(═O)O—R¹¹, —OC(═O)O—R¹¹, —NHC(═O)NH—R¹¹, —OC(═O)—R¹¹; —N(R¹⁰)₂, substituted or unsubstituted C₁-C₆alkyl, C₁-C₆fluoroalkyl, substituted or unsubstituted C₂-C₆alkenyl, substituted or unsubstituted C₂-C₆alkynyl, substituted or unsubstituted C₁-C₆alkoxy, C₁-C₆ fluoroalkoxy, substituted or unsubstituted C₁-C₆heteroalkyl, substituted or unsubstituted C₃-C₈cycloalkyl, substituted or unsubstituted heterocycloalkyl, substituted or unsubstituted aryl, and substituted or unsubstituted heteroaryl;
R¹⁰ is hydrogen, or a substituted or unsubstituted group selected from among C₁-C₆alkyl, C₁-C₆fluoroalkyl, C₁-C₆heteroalkyl, C₃-C₈cycloalkyl, C₂-C₈heterocycloalkyl, aryl, and heteroaryl;
R¹¹ is a substituted or unsubstituted group selected from among C₁-C₆alkyl, C₁-C₆fluoroalkyl, C₃-C₈cycloalkyl, C₂-C₈heterocycloalkyl, aryl, and heteroaryl;
R³ is hydrogen, halogen, substituted or unsubstituted C₁-C₆alkyl, substituted or unsubstituted C₂-C₆alkenyl, substituted or unsubstituted C₂-C₆alkynyl, substituted or unsubstituted C₁-C₆alkoxy, substituted or unsubstituted C₁-C₆fluoroalkoxy, substituted or unsubstituted C₁-C₆heteroalkyl, substituted or unsubstituted phenyl, or —X⁶—R⁶;
X⁶ is a C₁-C₆alkylene, C₁-C₆fluoroalkylene, C₂-C₆alkenylene, C₂-C₆heteroalkylene;
R⁶ is hydrogen, halogen, —CN, hydroxy, amino, C₁-C₆alkylamino, di(C₁-C₆alkyl)amino, C₁-C₆alkoxy, C₃-C₈cycloalkyl, C₂-C₈heterocycloalkyl, phenyl, heteroaryl, or —X⁷—R⁷
X⁷ is a bond, —O—, —S—, —S(═O)—, —S(═O)₂—, —NR^{a}—, —C(═O)—, —C(═O)O—, —OC(═O)—, —NHC(═O)—, —C(═O)NR^{a}—, —S(═O)₂NR^{a}—, —NHS(═O)₂—, —OC(═O)NR^{a}—, —NHC(═O)O—, —OC(═O)O—, —NHC(═O)NR^{a}—;
R⁷ is hydrogen, C₁-C₆alkyl, C₂-C₆alkenyl, C₁-C₆heteroalkyl, C₁-C₆haloalkyl, C₃-C₈cycloalkyl, cycloalkylalkyl, C₂-C₈heterocycloalkyl, heterocycloalkylalkyl, aryl, arylalkyl, heteroaryl, heteroarylalkyl,
R^{a} is selected from among hydrogen, C₁-C₆alkyl, C₂-C₆alkenyl, hydroxy, C₁-C₆alkoxy, C₁-C₆fluoroalkoxy, C₁-C₆heteroalkyl; or
R^{a} and R⁷ together with the N atom to which they are attached form a 5-, 6-, or 7-membered heterocycloalkyl;
or an active metabolite, pharmaceutically acceptable solvate, pharmaceutically acceptable salt, pharmaceutically acceptable N-oxide, or pharmaceutically acceptable prodrug thereof.

21. A method for treating an inflammatory skin condition in a subject in need thereof, comprising administering to the subject a composition containing a therapeutically effective amount of a selective inhibitor of histone deacetylase 8 activity.
